(19) United States
(12) Patent Application Publication
(10) Pub. No.: US 2025/0180991 A1
(43) Pub. Date: Jun. 5, 2025

(54) RESIST COMPOSITION, RESIST PATTERN FORMATION METHOD, COMPOUND, AND POLYMER COMPOUND

(71) Applicant: TOKYO OHKA KOGYO CO., LTD., Kawasaki-shi (JP)

(72) Inventors: Junichi Miyakawa, Kawasaki-shi (JP); Yohei Adachi, Kawasaki-shi (JP); Tetsuo Fujinami, Kawasaki-shi (JP); Shuichi Ishii, Kawasaki-shi (JP)

(21) Appl. No.: 18/843,880

(22) PCT Filed: Mar. 7, 2023
(86) PCT No.: PCT/JP2023/008591

§ 371 Date: Sep. 4, 2024

(30) Foreign Application Priority Data
Mar. 9, 2022 (JP) 2022-036420

(51) Int. Cl. G03F 7/039; C07C 309/12; C08F 220/28; G03F 7/004; G03F 7/038; G03F 7/30; H01L 21/027

(52) U.S. Cl. CPC G03F 7/0397; C07C 309/12; C08F 220/283; G03F 7/0046; G03F 7/0382; G03F 7/0388; G03F 7/30; H01L 21/0274; C07C 2603/74

(57) ABSTRACT

A resist composition which generates an acid upon light exposure and whose solubility in a developing solution is changed by an action of an acid, the resist composition including a resin component whose solubility in a developing solution is changed by an action of an acid, in which the resin component has a constitutional unit represented by General Formula (a0-1) in which R represents a hydrogen atom, an alkyl group having 1 to 5 carbon atoms, a halogen atom, or a halogenated alkyl group having 1 to 5 carbon atoms; Ya⁰ represents a single bond or a divalent linking group; Va⁰ represents a single bond or a linear or branched alkylene group; and M^{m+} represents an m-valent cation

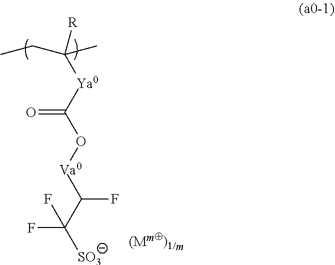

### TECHNICAL FIELD

The present invention relates to a resist composition, a resist pattern formation method, a compound, and a polymer compound.

Priority is claimed on Japanese Patent Application No. 2022-036420, filed Mar. 9, 2022, the content of which is incorporated herein by reference.

### BACKGROUND ART

In recent years, in the manufacture of semiconductor elements and liquid crystal display elements, advances in lithography technologies have led to rapid progress in the field of pattern miniaturization. These pattern miniaturization techniques typically involve shortening the wavelength (increasing the energy) of the exposure light source.

Resist materials require lithography characteristics such as a high resolution that enables reproduction of patterns with minute dimensions, and a high level of sensitivity to these kinds of exposure light sources.

As a resist material that satisfies these requirements, a chemically amplified resist composition containing a base material component whose solubility in a developing solution is changed by an action of an acid and an acid generator component that generates an acid upon light exposure has been used.

Further, a resin having a constitutional unit containing fluorine may be blended into the chemically amplified resist composition in order to impart water repellency to the resist film as appropriate. For example, Patent Document 1 discloses a resist composition containing a base material component whose solubility in a developing solution is changed by an action of an acid, an acid generator component which generates an acid by light exposure, and a resin which has a constitutional unit containing fluorine.

### CITATION LIST

### Patent Document

Patent Document 1: Japanese Unexamined Patent Application, First Publication No. 2021-092704

### SUMMARY OF INVENTION

### Technical Problem

With further advances in lithography technologies, rapid progress in the field of pattern fining is being achieved together with the expansion of application fields. Along with this, a technology that enables formation of a fine pattern having a satisfactory shape is required in a case where a semiconductor element or the like is manufactured. Therefore, the resist composition is required to further enhance the sensitivity and further improve lithography characteristics such as the roughness, the pattern shape, and defects.

The present invention has been made in consideration of the above-described circumstances, and an object thereof is to provide a resist composition capable of achieving high sensitivity and having satisfactory lithography characteristics, a resist pattern formation method using the resist composition, a polymer compound that can be used for producing the resist composition, and a compound that can be used for synthesizing the polymer.

### Solution to Problem

In order to achieve the above-described object, the present invention employs the following configurations.

That is, according to a first aspect of the present invention, there is provided a resist composition which generates an acid upon light exposure and whose solubility in a developing solution is changed by an action of an acid, the resist composition including: a resin component (A1) whose solubility in a developing solution is changed by an action of an acid, in which the resin component (A1) has a constitutional unit (a0) represented by General Formula (a0-1).

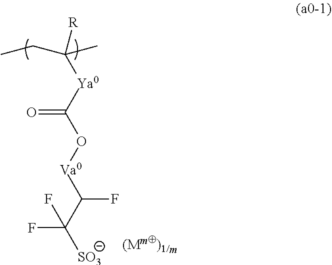

[In the formula, R represents a hydrogen atom, an alkyl group having 1 to 5 carbon atoms, a halogen atom, or a halogenated alkyl group having 1 to 5 carbon atoms. Ya⁰ represents a single bond or a divalent linking group. Va⁰ represents a single bond or a linear or branched alkylene group. m represents an integer of 1 or greater, and M^{m+} represents an m-valent cation.]

According to a second aspect of the present invention, there is provided a resist pattern formation method including: a step of forming a resist film on a support using the resist composition according to the first aspect; a step of exposing the resist film to light; and a step of developing the resist film exposed to light to form a resist pattern.

According to a third aspect of the present invention, there is provided a compound which is represented by General Formula (m0-1).

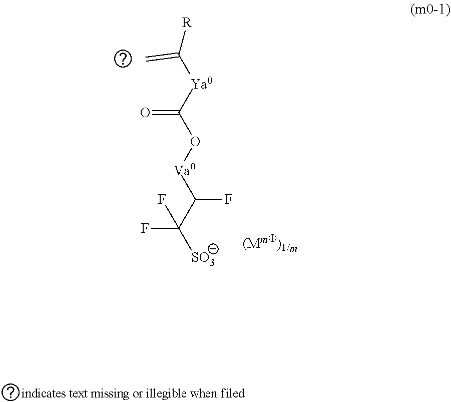

[In the formula, R represents a hydrogen atom, an alkyl group having 1 to 5 carbon atoms, a halogen atom, or a halogenated alkyl group having 1 to 5 carbon atoms. Ya⁰ represents a single bond or a divalent linking group. Va⁰ represents a single bond or a linear or branched alkylene group. m represents an integer of 1 or greater, and M^{m+} represents an m-valent cation.]

According to a fourth aspect of the present invention, there is provided a polymer compound which has a constitutional unit (a0) represented by General Formula (a0-1).

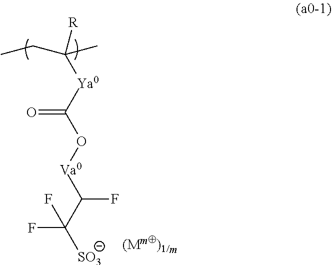

[In the formula, R represents a hydrogen atom, an alkyl group having 1 to 5 carbon atoms, a halogen atom, or a halogenated alkyl group having 1 to 5 carbon atoms. Ya⁰ represents a single bond or a divalent linking group. Va⁰ represents a single bond or a linear or branched alkylene group. m represents an integer of 1 or greater, and M^{m+} represents an m-valent cation.]

### Advantageous Effects of Invention

According to the present invention, it is possible to provide a resist composition capable of achieving high sensitivity and having satisfactory lithography characteristics, a resist pattern formation method using the resist composition, a polymer compound that can be used for producing the resist composition, and a compound that can be used for synthesizing the polymer.

### DESCRIPTION OF EMBODIMENTS

In the present specification and the scope of the present claims, the term “aliphatic” is a relative concept used in relation to the term “aromatic”, and defines a group, a compound, or the like that has no aromaticity.

The term “alkyl group” includes a linear, branched, or cyclic monovalent saturated hydrocarbon group unless otherwise specified. The same applies to the alkyl group in an alkoxy group.

The term “alkylene group” includes a linear, branched, or cyclic divalent saturated hydrocarbon group unless otherwise specified.

Examples of the “halogen atom” include a fluorine atom, a chlorine atom, a bromine atom, and an iodine atom.

The term “constitutional unit” indicates a monomer unit constituting a polymer compound (a resin, a polymer, or a copolymer).

The expression “may have a substituent” includes both a case where a hydrogen atom (—H) is substituted with a monovalent group and a case where a methylene (—CH₂—) group is substituted with a divalent group.

The term “light exposure” is a general concept for irradiation with radiation.

The term “acid-decomposable group” indicates a group having acid decomposability in which at least a part of a bond in the structure of the acid-decomposable group can be cleaved due to the action of an acid.

Examples of the acid-decomposable group whose polarity is increased by the action of an acid include groups which are decomposed by the action of an acid to generate a polar group.

Examples of the polar group include a carboxy group, a hydroxyl group, an amino group, and a sulfo group (—SO₃H).

More specific examples of the acid-decomposable group include a group in which the above-described polar group has been protected by an acid-dissociable group (such as a group in which a hydrogen atom of the OH-containing polar group has been protected by an acid-dissociable group).

Here, the term “acid-dissociable group” indicates both a group (i) having an acid dissociation property in which a bond between the acid-dissociable group and an atom adjacent to the acid-dissociable group can be cleaved due to the action of an acid and a group (ii) in which some bonds are cleaved due to the action of an acid, a decarboxylation reaction occurs, and thus the bond between the acid-dissociable group and the atom adjacent to the acid-dissociable group can be cleaved.

It is necessary that the acid-dissociable group that constitutes the acid-decomposable group be a group which exhibits a lower polarity than that of the polar group generated by the dissociation of the acid-dissociable group. Thus, in a case where the acid-dissociable group is dissociated by the action of an acid, a polar group exhibiting a higher polarity than that of the acid-dissociable group is generated so that the polarity is increased. As a result, the polarity of an entire component (A1) is increased. Due to the increase in the polarity, the solubility in a developing solution is relatively changed such that the solubility is increased in a case where the developing solution is an alkali developing solution and the solubility is decreased in a case where the developing solution is an organic developing solution.

The term “base material component” denotes an organic compound having a film-forming ability. Organic compounds used as the base material component are classified into non-polymers and polymers. As the non-polymers, those having a molecular weight of 500 or greater and less than 4000 are typically used. Hereinafter, the term “low-molecular-weight compound” denotes a non-polymer having a molecular weight of 500 or greater and less than 4000. As the polymer, those having a molecular weight of 1000 or greater are typically used. Hereinafter, “resin”, “polymer compound”, or “polymer” indicates a polymer having a molecular weight of 1000 or greater. As the molecular weight of the polymer, the weight-average molecular weight in terms of polystyrene according to gel permeation chromatography (GPC) is used.

The expression “constitutional unit to be derived” denotes a constitutional unit formed by cleavage of a multiple bond between carbon atoms, for example, an ethylenic double bond.

In “acrylic acid ester”, the hydrogen atom bonded to the carbon atom at the α-position may be substituted with a substituent. The substituent (R^{αx}) that substitutes the hydrogen atom bonded to the carbon atom at the α-position is an atom other than the hydrogen atom or a group. Further, the acrylic acid ester includes itaconic acid diester in which the substituent (R^{αx}) has been substituted with a substituent having an ester bond and α-hydroxyacryl ester in which the substituent (R^{αx}) has been substituted with a hydroxyalkyl group or a group obtained by modifying a hydroxyl group thereof. Further, the carbon atom at the α-position of acrylic acid ester indicates the carbon atom to which the carbonyl group of acrylic acid is bonded, unless otherwise specified.

Hereinafter, acrylic acid ester in which the hydrogen atom bonded to the carbon atom at the α-position has been substituted with a substituent is also referred to as α-substituted acrylic acid ester.

The concept “derivative” includes those obtained by substituting a hydrogen atom at the α-position of a target compound with another substituent such as an alkyl group or a halogenated alkyl group, and derivatives thereof. Examples of the derivatives thereof include those obtained by substituting a hydrogen atom of a hydroxyl group of a target compound, in which the hydrogen atom at the α-position may be substituted with a substituent, with an organic group, and those obtained by bonding a substituent other than a hydroxyl group to a target compound in which the hydrogen atom at the α-position may be substituted with a substituent. Further, the α-position denotes the first carbon atom adjacent to a functional group unless otherwise specified.

Examples of the substituent that substitutes the hydrogen atom at the α-position of hydroxystyrene include those for R^{αx}.

In the present specification and the scope of the present claims, asymmetric carbons may be present and enantiomers or diastereomers may be present depending on the structures of the chemical formulae. In this case, these isomers are represented by one chemical formula. These isomers may be used alone or in the form of a mixture.

### (Resist Composition)

The resist composition according to the present embodiment is a resist composition which generates an acid upon light exposure and whose solubility in a developing solution is changed by the action of the acid.

The resist composition contains a base material component (A) (hereinafter, also referred to as “component (A)”) whose solubility in a developing solution is changed by the action of the acid. In the resist composition according to the present embodiment, the component (A) generates an acid upon light exposure. It is preferable that the component (A1) described below in the resist composition according to the present embodiment be a resin which generates an acid upon light exposure and whose solubility in a developing solution is changed by an action of the acid.

In a case where a resist film is formed of the resist composition according to the present embodiment and the resist film is selectively exposed, since an acid is generated from the component (A) in an exposed portion of the resist film and the solubility of the component (A) in a developing solution is changed by the action of the acid while the solubility of the component (A) in a developing solution is not changed in an unexposed portion of the resist film, a difference in solubility in the developing solution occurs between the exposed portion and the unexposed portion. Therefore, in a case where the resist film is developed, the exposed portion of the resist film is dissolved and removed to form a positive-tone resist pattern in a case where the resist composition is of a positive tone, whereas the unexposed portion of the resist film is dissolved and removed to form a negative-tone resist pattern in a case where the resist composition is of a negative tone.

The resist composition of the present embodiment may be a positive-tone resist composition or a negative-tone resist composition. Further, the resist composition of the present embodiment may be used in an alkali developing process using an alkali developing solution in the developing treatment in a case of forming a resist pattern or may be used in a solvent developing process using a developing solution containing an organic solvent (organic developing solution) in the developing treatment.

### <Component (A)>

In the resist composition according to the present embodiment, the component (A) contains a resin component (A1) (hereinafter, also referred to as “component (A1)”) whose solubility in a developing solution is changed by the action of an acid.

Since the polarity of the base material component before and after the light exposure is changed by using the component (A1), an excellent development contrast can be obtained not only in an alkali developing process but also in a solvent developing process.

As the component (A), another polymer compound and/or a low-molecular-weight compound may be used in combination with the component (A1).

In the resist composition according to the present embodiment, the component (A) may be used alone or in combination of two or more kinds thereof.

### In Regard to Component (A1)

The component (A1) is a resin component whose solubility in a developing solution is changed by the action of an acid.

The component (A) has a constitutional unit (a0) represented by General Formula (a0-1).

Further, the component (A1) may have other constitutional units as necessary in addition to the constitutional unit (a0).

<<Constitutional Unit (a0)>>

The constitutional unit (a0) is a constitutional unit represented by General Formula (a0-1).

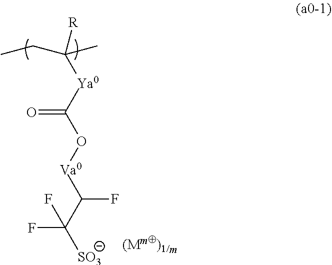

[In the formula, R represents a hydrogen atom, an alkyl group having 1 to 5 carbon atoms, a halogen atom, or a halogenated alkyl group having 1 to 5 carbon atoms. Ya⁰ represents a single bond or a divalent linking group. Va⁰ represents a single bond or a linear or branched alkylene group. m represents an integer of 1 or greater, and M^{m+} represents an m-valent cation.]

In General Formula (a0-1), R represents a hydrogen atom, an alkyl group having 1 to 5 carbon atoms, a halogen atom, or a halogenated alkyl group having 1 to 5 carbon atoms.

As the alkyl group having 1 to 5 carbon atoms as R, a linear or branched alkyl group having 1 to 5 carbon atoms is preferable, and a linear or branched alkyl group having 1 to 3 carbon atoms is more preferable. Specific examples thereof include a methyl group, an ethyl group, a propyl group, an isopropyl group, an n-butyl group, an isobutyl group, a tert-butyl group, a pentyl group, an isopentyl group, and a neopentyl group.

Examples of the halogen atom include a fluorine atom, a bromine atom, and an iodine atom. Among these, a fluorine atom is preferable.

The halogenated alkyl group having 1 to 5 carbon atoms is a group in which some or all hydrogen atoms in the alkyl group having 1 to 5 carbon atoms have been substituted with halogen atoms. As the halogen atom, a fluorine atom is particularly preferable.

R represents preferably a hydrogen atom, an alkyl group having 1 to 3 carbon atoms, or a fluorinated alkyl group having 1 to 3 carbon atoms and more preferably a hydrogen atom or a methyl group from the viewpoint of industrial availability.

In General Formula (a0-1), Ya⁰ represents a single bond or a divalent linking group. Examples of the divalent linking group as Ya⁰ include a divalent hydrocarbon group which may have a substituent and a divalent linking group having a heteroatom.

Divalent Hydrocarbon Group which May have Substituent:

In a case where Ya⁰ represents a divalent hydrocarbon group which may have a substituent, the hydrocarbon group may be an aliphatic hydrocarbon group or an aromatic hydrocarbon group.

### Aliphatic Hydrocarbon Group

The aliphatic hydrocarbon group indicates a hydrocarbon group with no aromaticity. The aliphatic hydrocarbon group may be saturated or unsaturated. In general, it is preferable that the aliphatic hydrocarbon group be saturated.

Examples of the aliphatic hydrocarbon group include a linear or branched aliphatic hydrocarbon group and an aliphatic hydrocarbon group having a ring in the structure thereof.

### Linear or Branched Aliphatic Hydrocarbon Group

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

The linear or branched aliphatic hydrocarbon group may or may not have a substituent. Examples of the substituent include a fluorine atom, a fluorinated alkyl group having 1 to 5 carbon atoms which has been substituted with a fluorine atom, and a carbonyl group.

### Aliphatic Hydrocarbon Group Having Ring in Structure Thereof

Examples of the aliphatic hydrocarbon group having a ring in the structure thereof include a cyclic aliphatic hydrocarbon group which may have a substituent having a heteroatom in the ring structure thereof (a group in which two hydrogen atoms have been removed from an aliphatic hydrocarbon ring), a group in which the cyclic aliphatic hydrocarbon group is bonded to the terminal of a linear or branched aliphatic hydrocarbon group, and a group in which the cyclic aliphatic hydrocarbon group is interposed in the middle of a linear or branched aliphatic hydrocarbon group. Examples of the linear or branched aliphatic hydrocarbon group include the same groups as those described above.

The cyclic aliphatic hydrocarbon group has preferably 3 to 20 carbon atoms and more preferably 3 to 12 carbon atoms.

The cyclic aliphatic hydrocarbon group may be a polycyclic group or a monocyclic group. As the monocyclic alicyclic hydrocarbon group, a group in which two hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. As the polycycloalkane, a group having 7 to 12 carbon atoms is preferable. Specific examples of the polycycloalkane include adamantane, norbornane, isobornane, tricyclodecane, and tetracyclododecane.

The cyclic aliphatic hydrocarbon group may or may not have a substituent. Examples of the substituent include an alkyl group, an alkoxy group, a halogen atom, a halogenated alkyl group, a hydroxyl group, and a carbonyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is still more preferable.

As the halogen atom as the substituent, a fluorine atom is preferable.

Examples of the halogenated alkyl group as the substituent include groups in which some or all hydrogen atoms in the above-described alkyl groups are substituted with the above-described halogen atoms.

In the cyclic aliphatic hydrocarbon group, some carbon atoms constituting the ring structure thereof may be substituted with a substituent having a heteroatom. As the substituent having a heteroatom, —O—, —C(═O)—O—, —S—, —S(═O)₂—, or —S(═O)₂—O— is preferable.

### Aromatic Hydrocarbon Group

The aromatic hydrocarbon group is a hydrocarbon group having at least one aromatic ring.

The aromatic ring is not particularly limited as long as it is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms. Here, the number of carbon atoms in a substituent is not included in the number of carbon atoms.

Specific examples of the aromatic ring include aromatic hydrocarbon rings such as benzene, naphthalene, anthracene, and phenanthrene; and aromatic heterocyclic rings in which some carbon atoms constituting the above-described aromatic hydrocarbon rings have been substituted with heteroatoms. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group include a group in which two hydrogen atoms have been removed from the above-described aromatic hydrocarbon ring or aromatic heterocyclic ring (an arylene group or a heteroarylene group); a group in which two hydrogen atoms have been removed from an aromatic compound having two or more aromatic rings (such as biphenyl or fluorene); and a group in which one hydrogen atom of a group (an aryl group or a heteroaryl group) obtained by removing one hydrogen atom from the above-described aromatic hydrocarbon ring or aromatic heterocyclic ring has been substituted with an alkylene group (for example, a group obtained by further removing one hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aryl group or the heteroaryl group is preferably in a range of 1 to 4, more preferably 1 or 2, and particularly preferably 1.

In the aromatic hydrocarbon group, the hydrogen atom in the aromatic hydrocarbon group may be substituted with a substituent. For example, the hydrogen atom bonded to the aromatic ring in the aromatic hydrocarbon group may be substituted with a substituent. Examples of substituents include an alkyl group, an alkoxy group, a halogen atom, a halogenated alkyl group, and a hydroxyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group, the halogen atom, and the halogenated alkyl group as the substituents, the groups described as the substituents that substitute a hydrogen atom in the cyclic aliphatic hydrocarbon group are exemplary examples.

### Divalent Linking Group Having Heteroatom:

In a case where Ya⁰ represents a divalent linking group having a heteroatom, preferred examples of the linking group include —O—, —C(═O)—O—, —O—C(═O)—, —C(═O)—, —O—C(═O)—O—, —C(═O)—NH—, —NH—, —NH—C(═NH)— (H may be substituted with a substituent such as an alkyl group or an acyl group), —S—, —S(═O)₂—, —S(═O)₂—O—, and a group represented by General Formula: —Y²¹—O—Y², —Y²¹—O—, —Y²¹—C(═O)—O—, —C(═O)—O—Y²¹, —[Y²¹—C(═O)—O]_{m″}—Y²²—, —Y²¹—O—C(═O)—Y²²—, or —Y²¹—S(═O)₂—O—Y²²— [in the formulae, Y²¹ and Y²² each independently represents a divalent hydrocarbon group which may have a substituent, O represents an oxygen atom, and m″ represents an integer of 0 to 3].

In a case where the above-described divalent linking group containing a heteroatom is —C(═O)—NH—, —C(═O)—NH—C(═O)—, —NH—, or —NH—C(═NH)—, H may be substituted with a substituent such as an alkyl group and an acyl group. The substituent (an alkyl group, an acyl group, or the like) has preferably 1 to 10 carbon atoms, more preferably 1 to 8 carbon atoms, and particularly preferably 1 to 5 carbon atoms.

In General Formula —Y²¹—O—Y²²—, —Y²¹—O—, —Y²¹—C(═O)—O—, —C(═O)—O—Y²¹—, —[Y²¹—C(═O)—O]_{m″}—Y²²—, —Y²¹—O—C(═O)—Y²²—, or —Y²¹—S(═O)₂—O—Y²²—, Y²¹ and Y²² each independently represent a divalent hydrocarbon group which may have a substituent. As the divalent hydrocarbon group, the same ones as those described above are exemplary examples.

Y²¹ represents preferably a linear aliphatic hydrocarbon group, more preferably a linear alkylene group, still more preferably a linear alkylene group having 1 to 5 carbon atoms, and particularly preferably a methylene group or an ethylene group.
Y²² represents preferably a linear or branched aliphatic hydrocarbon group and more preferably a methylene group, an ethylene group, or an alkylmethylene group. The alkyl group in the alkylmethylene group is preferably a linear alkyl group having 1 to 5 carbon atoms, more preferably a linear alkyl group having 1 to 3 carbon atoms, and most preferably a methyl group.

In the group represented by Formula —[Y²¹—C(═O)—O]_{m″}—Y²²—, m″ represents an integer of 0 to 3, preferably an integer of 0 to 2, more preferably 0 or 1, and particularly preferably 1. That is, a group represented by Formula —Y²¹—C(═O)—O—Y²²— is particularly preferable as the group represented by Formula —[Y²¹—C(═O)—O]_{m″}—Y²²—. Among these, a group represented by Formula —(CH₂)_{a′}—C(═O)—O—(CH₂)_{b′}— is preferable. In the formula, a′ represents an integer of 1 to 10, preferably an integer of 1 to 8, more preferably an integer of 1 to 5, still more preferably 1 or 2, and most preferably 1. b′ represents an integer of 1 to 10, preferably an integer of 1 to 8, more preferably an integer of 1 to 5, still more preferably 1 or 2, and most preferably 1.

As the divalent linking group as Ya⁰, a hydrocarbon group which may have a substituent or a hydrocarbon group which may have a substituent having an ester bond [—C(═O)—O— or —O—C(═O)—] is preferable, and an aromatic hydrocarbon group or —C(═O)—O—Y²¹— is more preferable. It is preferable that Y²¹ represent a linear or branched alkyl group, an alicyclic group, a lactone-containing cyclic group, or a —SO₂— containing cyclic group. As the linear or branched alkylene group, a linear alkylene group is preferable, and a linear alkylene group having 1 to 3 carbon atoms is more preferable. A polycyclic group is preferable as the alicyclic group.

In General Formula (a0-1), Va⁰ represents a single bond or a linear or branched alkylene group. The linear alkylene group as Va⁰ has preferably 1 to 10 carbon atoms, more preferably 1 to 6 carbon atoms, still more preferably 1 to 3 carbon atoms, and particularly preferably a methylene group or an ethylene group. The branched alkyl group as Va⁰ has preferably 2 to 12 carbon atoms, more preferably 2 to 6 carbon atoms, still more preferably 2 to 4 carbon atoms, and particularly preferably 2 or 3 carbon atoms.

Va⁰ represents preferably a linear or branched alkylene group and more preferably a linear alkylene group.

A constitutional unit represented by General Formula (a0-1-1) is preferable as the constitutional unit (a0).

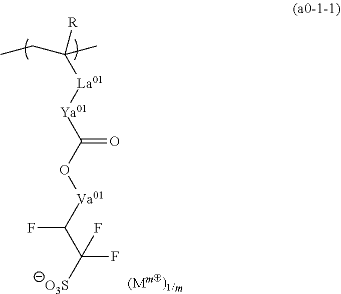

[In the formula, R represents a hydrogen atom, an alkyl group having 1 to 5 carbon atoms, a halogen atom, or a halogenated alkyl group having 1 to 5 carbon atoms. La⁰¹ represents a single bond, —COO—, or an aromatic hydrocarbon group which may have a substituent. Ya⁰¹ represents a single bond or a divalent linking group. Va⁰¹¹ represents a single bond or a linear or branched alkylene group. m represents an integer of 1 or greater, and M^{m+} represents an m-valent cation.]

In General Formula (a0-1-1), R has the same definition as that for R in General Formula (a0-1).

In General Formula (a0-1-1), La⁰¹ represents —COO— or an aromatic hydrocarbon group which may have a substituent.

Examples of the aromatic hydrocarbon group as La⁰¹ include the same groups as those for the aromatic hydrocarbon group represented by Ya⁰ in General Formula (a0-1). As the aromatic hydrocarbon group in La⁰¹, a group obtained by removing two hydrogen atoms from an aromatic hydrocarbon ring or an aromatic heterocyclic ring is preferable, a group obtained by removing two hydrogen atoms from an aromatic hydrocarbon ring is more preferable, and a group obtained by removing two hydrogen atoms from benzene or naphthalene is still more preferable.

The aromatic hydrocarbon group as La⁰¹ may have a substituent. Examples of the substituent include the same substituents described as the substituent of the aromatic hydrocarbon group as Ya⁰ in General Formula (a0-1).

In General Formula (a0)-1-1), Ya⁰¹ represents a single bond or a divalent linking group. Examples of the divalent linking group as Ya⁰¹ include the same groups as those for the divalent linking group as Ya⁰ in General Formula (a0-1). As the divalent linking group represented by Ya⁰¹, a cyclic group which may have a substituent, a linear or branched alkylene group which may have a substituent, or a linear or branched alkenylene group which may have a substituent is preferable.

As the cyclic group which may have a substituent represented by Ya⁰¹, a cyclic hydrocarbon group is preferable. As the cyclic hydrocarbon group, an aliphatic hydrocarbon group having a ring in the structure is preferable. Examples of the aliphatic hydrocarbon group having a ring in the structure include the same groups as those for the aliphatic hydrocarbon group having a ring in the structure as Ya⁰. As the aliphatic hydrocarbon group having a ring in the structure as Ya⁰¹, a polycyclic group is preferable. The polycyclic alicyclic hydrocarbon group is preferably a group obtained by eliminating two hydrogen atoms from a polycycloalkane. The polycycloalkane has preferably 7 to 30 carbon atoms. Preferred examples of the polycycloalkane include a polycycloalkane having a crosslinked ring polycyclic skeleton such as adamantane, norbornane, isobornane, tricyclodecane, or tetracyclododecane; and a polycycloalkane having a condensed ring polycyclic skeleton such as a cyclic group having a steroid skeleton.

The cyclic hydrocarbon group as Ya⁰¹ may have a heteroatom such as a heterocyclic ring. Examples thereof include a lactone-containing cyclic group represented by any of General Formulae (a0-r-1) to (a0-r-7), a —SO₂-containing cyclic group represented by any of General Formulae (a0-s-1) to (a0-s-4), and a group in which one hydrogen atom has been removed from a heterocyclic group represented by any of Formulae (r-hr-1) to (r-hr-16).

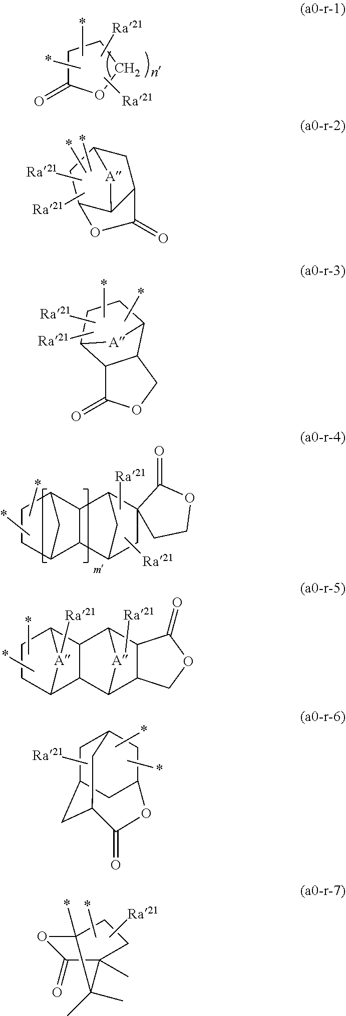

[In the formulae, Ra′²¹'s each independently represents a hydrogen atom, an alkyl group, an alkoxy group, a halogen atom, a halogenated alkyl group, a hydroxyl group, —COOR″, —OC(═O)R″, a hydroxyalkyl group, or a cyano group, R″ represents a hydrogen atom, an alkyl group, or a lactone-containing cyclic group, A″ represents an alkylene group having 1 to 5 carbon atoms which may have an oxygen atom (—O—) or a sulfur atom (—S—), an oxygen atom, or a sulfur atom, n′ represents an integer of 0 to 2, and m′ is 0 or 1. * represents a bonding site.]

Ra′²¹, A″, m′, and n′ in General Formulae (a0-r-1) to (a0-r-7) each has the same definition as that for Ra′²¹, A″, m′, and n′ in General Formulae (a2-r-1) to (a2-r-7).

Specific examples of the lactone-containing cyclic group as Ya⁰¹ include groups in which one hydrogen atom has been removed from the lactone ring represented by any of Formulae (r-lc-1-1) to (r-lc-1-7), (r-lc-2-1) to (r-lc-2-18), (r-lc-3-1) to (r-lc-3-5), (r-lc-4-1) to (r-lc-4-9), (r-lc-5-1) to (r-lc-5-4), (r-lc-6-1), and (r-lc-7-1).

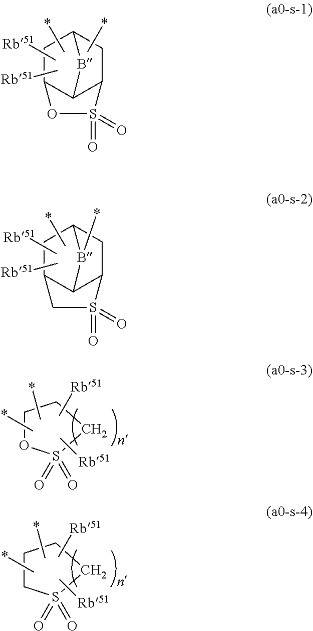

[In the formulae, Rb′⁵¹'s each independently represents a hydrogen atom, an alkyl group, an alkoxy group, a halogen atom, a halogenated alkyl group, a hydroxyl group, —COOR″, —OC(═O)R″, a hydroxyalkyl group, or a cyano group, R″ represents a hydrogen atom, an alkyl group, a lactone-containing cyclic group, or a —SO₂-containing cyclic group, B″ represents an alkylene group having 1 to 5 carbon atoms which may have an oxygen atom or a sulfur atom, an oxygen atom, or a sulfur atom, and n′ represents an integer of 0 to 2. * represents a bonding site.]

In General Formulae (a0-s-1) and (a0-s-2), B″ represents an alkylene group having 1 to 5 carbon atoms, which may have an oxygen atom or a sulfur atom, an oxygen atom, or a sulfur atom. B″ represents preferably an alkylene group having 1 to 5 carbon atoms or —O—, more preferably an alkylene group having 1 to 5 carbon atoms, and still more preferably a methylene group.

In General Formulae (a0-s-1) to (a0-s-4), Rb′⁵¹'s each independently represents a hydrogen atom, an alkyl group, an alkoxy group, a halogen atom, a halogenated alkyl group, a hydroxyl group, —COOR″, —OC(═O)R″, a hydroxyalkyl group, or a cyano group. Among these, it is preferable that Rb′⁵¹'s each independently represent a hydrogen atom or a cyano group.

Specific examples of the —SO₂-containing cyclic group as Ya⁰¹ include groups in which one hydrogen atom has been removed from the —SO₂-containing ring of a group represented by any of Formulae (r-sl-1-1) to (r-sl-1-33) and (r-sl-2-1) to (r-sl-2-4).

The cyclic group as Ya⁰¹ may or may not have a substituent. Examples of the substituent include the same substituents described as the substituent of the aliphatic hydrocarbon group having a ring in the structure as Ya⁰¹. Examples of the substituent include an alkyl group, an alkoxy group, a halogen atom, a halogenated alkyl group, a hydroxyl group, a carbonyl group, and a nitro group. The number of carbon atoms in the alkyl group, the alkoxy group, and the halogenated alkyl group as the substituent is preferably in a range of 1 to 5.

The linear alkylene group as Ya⁰¹ has preferably 1 to 10 carbon atoms, more preferably 1 to 6 carbon atoms, still more preferably 1 to 4 carbon atoms, and particularly preferably 1, 2, or 3 carbon atoms.

The branched alkylene group has preferably 2 to 10 carbon atoms, more preferably 2 to 6 carbon atoms, and still more preferably 2 to 4 carbon atoms.

The linear or branched alkenylene group as Ya⁰¹ has preferably 3 to 10 carbon atoms, more preferably 3 to 5 carbon atoms, and still more preferably 3 or 4 carbon atoms.

The alkylene group or alkenylene group as Ya⁰¹ may or may not have a substituent. Examples of the substituent include the same substituents described as the substituent of the linear or branched aliphatic hydrocarbon group as Ya⁰. Examples of the substituent of the cyclic group include an alkoxy group, a halogen atom, a halogenated alkyl group, a hydroxyl group, a carbonyl group, a nitro group, an amino group, and the cyclic group as Ya⁰¹.

It is preferable that Ya⁰¹ represent a divalent linking group having a polycyclic structure.

Ya⁰¹ represents preferably a cyclic group which may have a substituent and more preferably a cyclic hydrocarbon group which may have a substituent. As the cyclic hydrocarbon group, a group in which two hydrogen atoms have been removed from benzene, naphthalene, or polycycloalkane, a lactone-containing cyclic group represented by any of General Formulae (a0-r-1) to (a0-r-7), or a —SO₂-containing cyclic group represented by any of General Formulae (a0-s-1) to (s0-s-4) is preferable, a group in which two hydrogen atoms have been removed from polycycloalkane is more preferable, and a group in which two hydrogen atoms have been removed from adamantane is still more preferable.

In General Formula (a0-1-1), Va⁰¹ represents a single bond or a linear or branched alkylene group. Va⁰¹ has the same definition as that for Va⁰ in General Formula (a0-1).

Specific examples of the constitutional unit (a0) are shown below, but the examples are not limited thereto. In the formulae shown below, R^{α} represents a hydrogen atom, a methyl group, or a trifluoromethyl group. M^{m+} represents an m-valent cation.

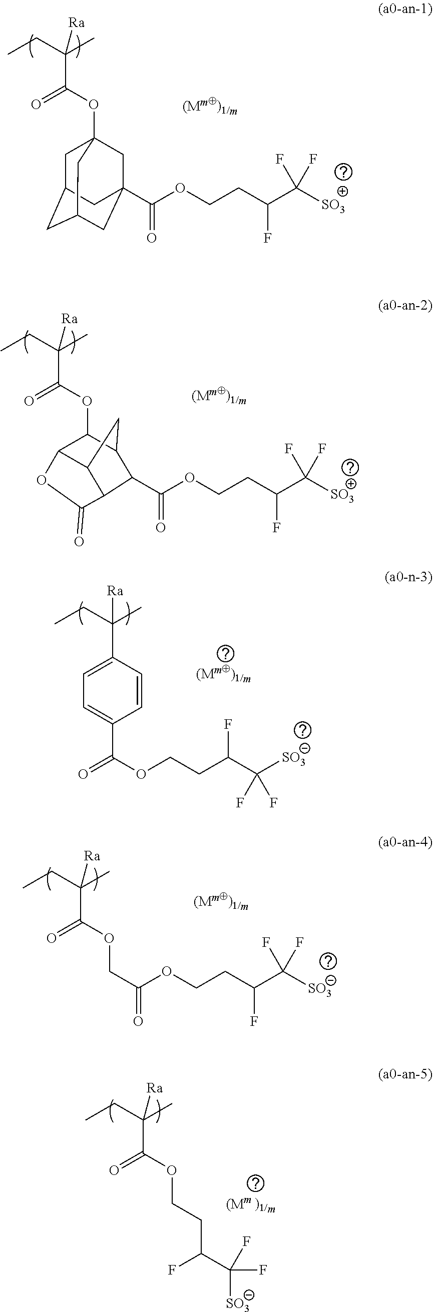

### {Cation Moiety}

In the formulae shown above, M^{m+} represents an m-valent cation. A sulfonium cation or an iodonium cation is preferable as the cation. m represents an integer of 1 or greater.

Preferred examples of the cation moiety ((M^{m+})_{1/m}) include organic cations each represented by General Formulae (ca-1) to (ca-3).

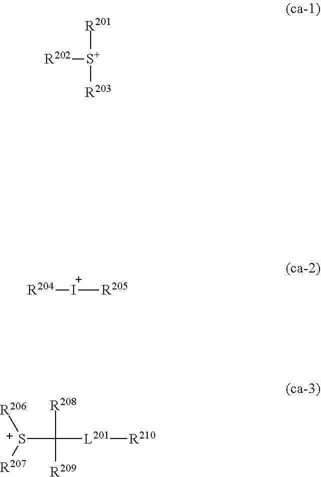

[In the formulae, R²⁰¹ to R²⁰⁷ each independently represents an aryl group, an alkyl group, or an alkenyl group, which may have a substituent. R²⁰¹ to R²⁰³, and R²⁰⁶ and R²⁰⁷ may be bonded to each other to form a ring with the sulfur atoms in the formulae. R²⁰⁸ and R²⁰⁹ each independently represents a hydrogen atom or an alkyl group having 1 to 5 carbon atoms. R²¹⁰ represents an aryl group which may have a substituent, an alkyl group which may have a substituent, an alkenyl group which may have a substituent, or a —SO₂-containing cyclic group which may have a substituent. L²⁰¹ represents —C(═O)— or —C(═O)—O—.]

In General Formulae (ca-1) to (ca-3), examples of the aryl group as R²⁰¹ to R²⁰⁷ include an unsubstituted aryl group having 6 to 20 carbon atoms. Among these, a phenyl group or a naphthyl group is preferable.

As the alkyl group as R²⁰¹ to R²⁰⁷, a chain-like or cyclic alkyl group having 1 to 30 carbon atoms is preferable.

It is preferable that the alkenyl group as R²⁰¹ to R²⁰⁷ have 2 to 10 carbon atoms. Examples of the substituent that R²⁰¹ to R²⁰⁷ and R²¹⁰ may have include an alkyl group, a halogen atom, a halogenated alkyl group, a carbonyl group, a cyano group, an amino group, an aryl group, and groups each represented by General Formulae (ca-r-1) to (ca-r-7).

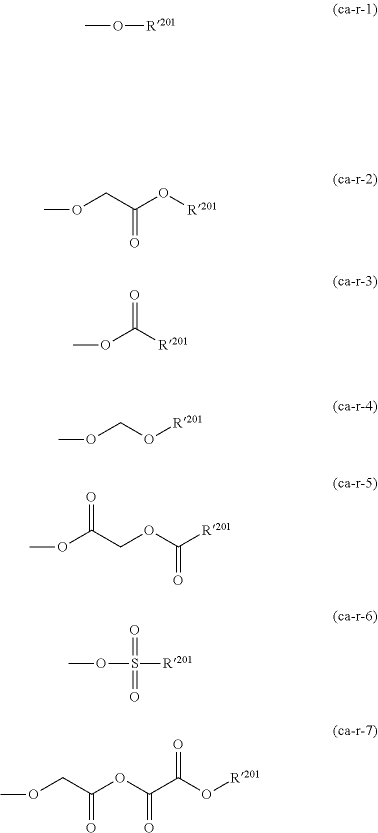

[In the formulae, R′²⁰¹'s each independently represents a hydrogen atom, a cyclic group which may have a substituent, a chain-like alkyl group which may have a substituent, or a chain-like alkenyl group which may have a substituent.]

Cyclic group which may have substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group with no aromaticity. Further, the aliphatic hydrocarbon group may be saturated or unsaturated. In general, it is preferable that the aliphatic hydrocarbon group be saturated.

The aromatic hydrocarbon group as R′²⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring contained in the aromatic hydrocarbon group as R′²⁰¹ include benzene, fluorene, naphthalene, anthracene, phenanthrene, biphenyl, or an aromatic heterocyclic ring in which some carbon atoms constituting any of these aromatic rings are substituted with heteroatoms. Examples of the heteroatom in the aromatic heterocyclic rings include an oxygen atom, a sulfur atom, and a nitrogen atom.

Specific examples of the aromatic hydrocarbon group as R′²⁰¹ include a group in which one hydrogen atom has been removed from the aromatic ring (an aryl group such as a phenyl group or a naphthyl group), and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R′²⁰¹ include an aliphatic hydrocarbon group having a ring in the structure thereof.

Examples of the aliphatic hydrocarbon group having a ring in the structure thereof include an alicyclic hydrocarbon group (a group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, tricyclodecane, or tetracyclododecane; and a polycycloalkane having a condensed ring polycyclic skeleton such as a cyclic group having a steroid skeleton are preferable as the polycycloalkane.

Among these examples, as the cyclic aliphatic hydrocarbon group as R′²⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is particularly preferable, and an adamantyl group is most preferable.

The linear or branched aliphatic hydrocarbon group which may be bonded to the alicylic hydrocarbon group has preferably 1 to 10 carbon atoms, more preferably 1 to 6 carbon atoms, still more preferably 1 to 4 carbon atoms, and particularly preferably 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

As the branched aliphatic hydrocarbon group, a branched alkylene group is preferable, and specific examples thereof include alkylalkylene groups, for example, alkylmethylene groups such as —CH(CH₃)—, —CH(CH₂CH)—, —C(CH₃)—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

Further, the cyclic hydrocarbon group as R′²⁰¹ may have a heteroatom such as a heterocyclic ring. Specific examples thereof include lactone-containing cyclic groups each represented by General Formulae (a2-r-1) to (a2-r-7), —SO₂-containing cyclic group each represented by General Formulae (b5-r-1) to (b5-r-4), and other heterocyclic groups each represented by Chemical Formulae (r-hr-1) to (r-hr-16).

Examples of the substituent for the cyclic group as R′²⁰¹ include an alkyl group, an alkoxy group, a halogen atom, a halogenated alkyl group, a hydroxyl group, a carbonyl group, and a nitro group.

As the alkyl group as the substituent, an alkyl group having 1 to 5 carbon atoms is preferable, and a methyl group, an ethyl group, a propyl group, an n-butyl group, or a tert-butyl group is most preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

As the halogen atom as a substituent, a fluorine atom is preferable.

Examples of the above-described halogenated alkyl group as the substituent include a group in which some or all hydrogen atoms in an alkyl group having 1 to 5 carbon atoms such as a methyl group, an ethyl group, a propyl group, an n-butyl group, or a tert-butyl group are substituted with the above-described halogen atoms.

The carbonyl group as the substituent is a group that substitutes a methylene group (—CH₂—) constituting the cyclic hydrocarbon group.

Chain-like alkyl group which may have substituent:

The chain-like alkyl group as R′²⁰¹ may be linear or branched.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-like alkenyl group which may have substituent:

The chain-like alkenyl group as R′²⁰¹ may be linear or branched, and the number of carbon atoms thereof is preferably in a range of 2 to 10, more preferably in a range of 2 to 5, still more preferably in a range of 2 to 4, and particularly preferably 3. Examples of the linear alkenyl group include a vinyl group, a propenyl group (an allyl group), and a butynyl group. Examples of the branched alkenyl group include a 1-methylvinyl group, a 2-methylvinyl group, a 1-methylpropenyl group, and a 2-methylpropenyl group.

Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R′²⁰¹ include an alkoxy group, a halogen atom, a halogenated alkyl group, a hydroxyl group, a carbonyl group, a nitro group, an amino group, and a cyclic group as R′²⁰¹.

Examples of the cyclic group which may have a substituent, the chain-like alkyl group which may have a substituent, and the chain-like alkenyl group which may have a substituent as R′²⁰¹ include those for the acid-dissociable group represented by Formula (a1-r-2) which are the exemplary examples of the cyclic group which may have a substituent and the chain-like alkyl group which may have a substituent, in addition to those described above.

Among the examples, R′²⁰¹ represents preferably a cyclic group which may have a substituent and more preferably a cyclic hydrocarbon group which may have a substituent. More specific preferred examples thereof include a phenyl group, a naphthyl group, a group in which one or more hydrogen atoms have been removed from a polycycloalkane, a lactone-containing cyclic group represented by any of General Formulae (a2-r-1) to (a2-r-7), and a —SO₂-containing cyclic group represented by any of General Formulae (b5-r-1) to (b5-r-4).

In General Formulae (ca-1) to (ca-3), in a case where R²⁰¹ to R²⁰³ and R²⁰⁶ and R²⁰⁰⁷ are bonded to each other to form a ring with a sulfur atom in the formula, these groups may be bonded to each other via a heteroatom such as a sulfur atom, an oxygen atom, or a nitrogen atom, or a functional group such as a carbonyl group, —SO—, —SO₂—, —SO₃—, —COO—, —CONH— or —N(R_{N})— (R_{N} represents an alkyl group having 1 to 5 carbon atoms). As a ring to be formed, a ring containing the sulfur atom in the formula in the ring skeleton thereof is preferably a 3- to 10-membered ring and particularly preferably a 5- to 7-membered ring containing the sulfur atom. Specific examples of the ring to be formed include a thiophene ring, a thiazole ring, a benzothiophene ring, a dibenzothiophene ring, a 9H-thioxanthene ring, a thioxanthone ring, a thianthrene ring, a phenoxathiin ring, a tetrahydrothiophenium ring, and a tetrahydrothiopyranium ring.

R²⁰⁸ and R²⁰⁹ each independently represents a hydrogen atom or an alkyl group having 1 to 5 carbon atoms and preferably a hydrogen atom or an alkyl group having 1 to 3 carbon atoms. In a case where R²⁰⁸ and R²⁰⁹ represent an alkyl group, R²⁰⁸ and R²⁰⁹ may be bonded to each other to form a ring.

R²¹⁰ represents an aryl group which may have a substituent, an alkyl group which may have a substituent, an alkenyl group which may have a substituent, or a —SO₂-containing cyclic group which may have a substituent.

Examples of the aryl group as R²¹⁰ include an unsubstituted aryl group having 6 to 20 carbon atoms. Among these, a phenyl group or a naphthyl group is preferable.

As the alkyl group as R²¹⁰, a chain-like or cyclic alkyl group having 1 to 30 carbon atoms is preferable.

It is preferable that the alkenyl group as R²¹⁰ have 2 to 10 carbon atoms.

The —SO₂-containing cyclic group which may have a substituent as R²¹⁰ is preferably “—SO₂-containing polycyclic group” and more preferably a group represented by General Formula (b5-r-1).

Specific suitable examples of the cation represented by Formula (ca-D) include cations each represented by Chemical Formulae (ca.1.1) to (ca-1-75).

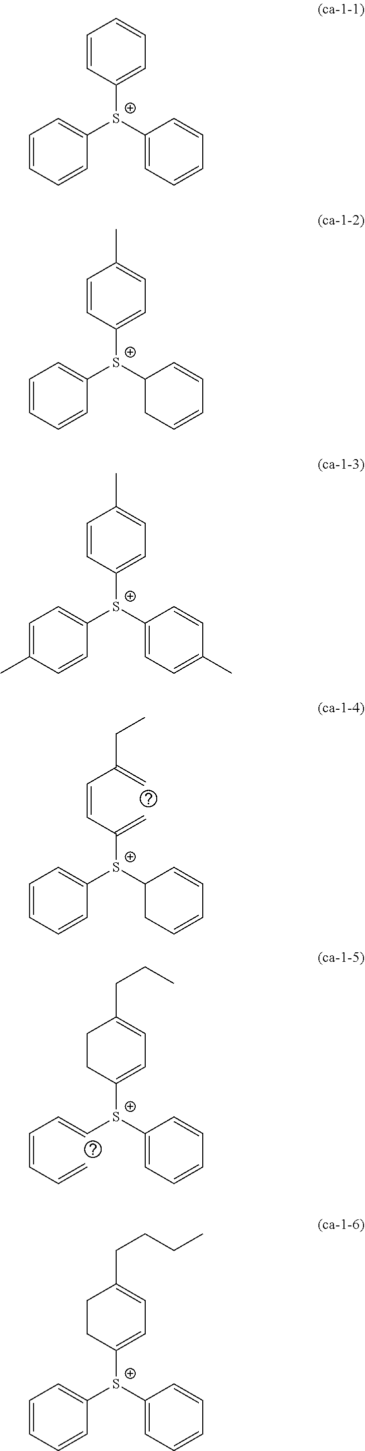

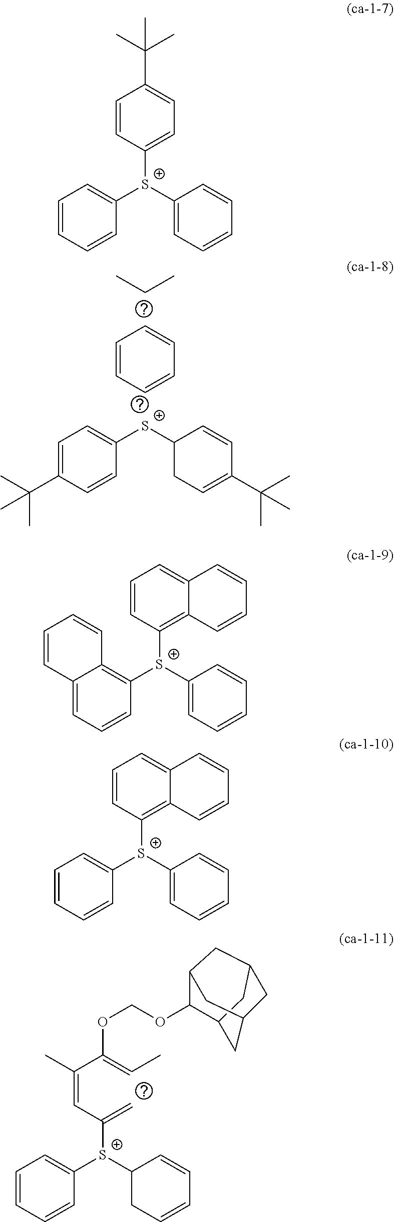

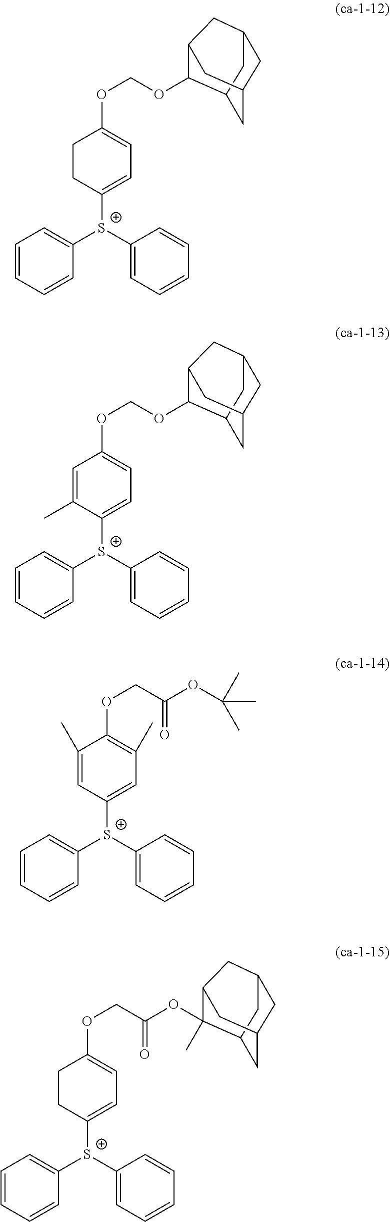 [SMILES: C1=C(OCOC2C3CC4CC(C3)CC2C4)CCC([S+](c2ccccc2)c2ccccc2)=C1 ; Cc1cc(OCOC2C3CC4CC(C3)CC2C4)ccc1[S+](c1ccccc1)c1ccccc1 ; Cc1cc([S+](c2ccccc2)c2ccccc2)cc(C)c1OCC(=O)OC(C)(C)C ; CC1(OC(=O)COC2=CC=C([S+](c3ccccc3)c3ccccc3)CC2)C2CC3CC(C2)CC1C3]

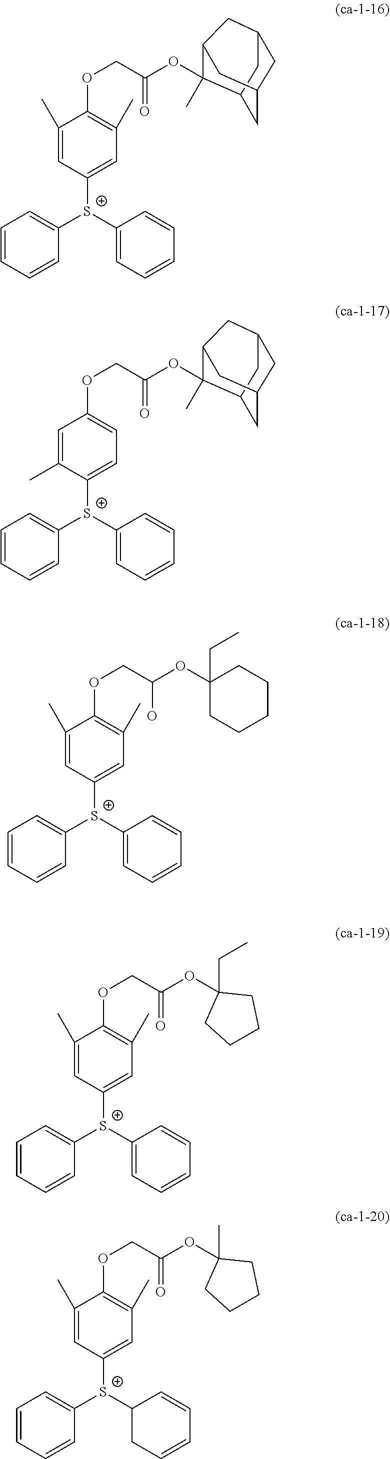 [SMILES: Cc1cc([S+](c2ccccc2)c2ccccc2)cc(C)c1OCC(=O)OC1(C)C2CC3CC(C2)CC1C3 ; Cc1cc(OCC(=O)OC2(C)C3CC4CC(C3)CC2C4)ccc1[S+](c1ccccc1)c1ccccc1 ; CCC1(OC(O)COc2c(C)cc([S+](c3ccccc3)c3ccccc3)cc2C)CCCCC1 ; CCC1(OC(=O)COc2c(C)cc([S+](c3ccccc3)c3ccccc3)cc2C)CCCC1 ; Cc1cc([S+](c2ccccc2)C2C=CC=CC2)cc(C)c1OCC(=O)OC1(C)CCCC1]

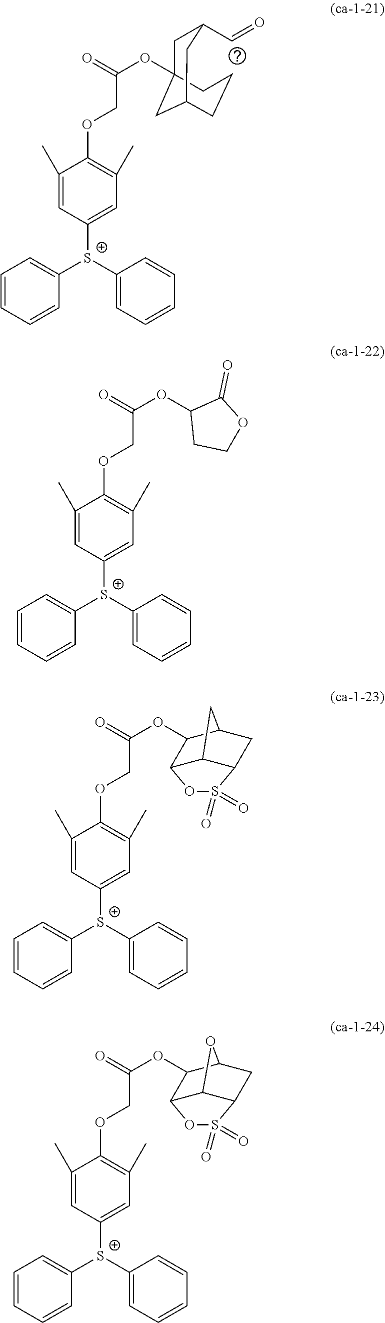 [SMILES: Cc1cc([S+](c2ccccc2)c2ccccc2)cc(C)c1OCC(=O)OC12CCCC(CC(C=O)C1)C2 ; Cc1cc([S+](c2ccccc2)c2ccccc2)cc(C)c1OCC(=O)OC1CCOC1=O ; Cc1cc([S+](c2ccccc2)c2ccccc2)cc(C)c1OCC(=O)OC1C2CC3C1OS(=O)(=O)C3C2 ; Cc1cc([S+](c2ccccc2)c2ccccc2)cc(C)c1OCC(=O)OC1C2CC3C(O2)C1OS3(=O)=O]

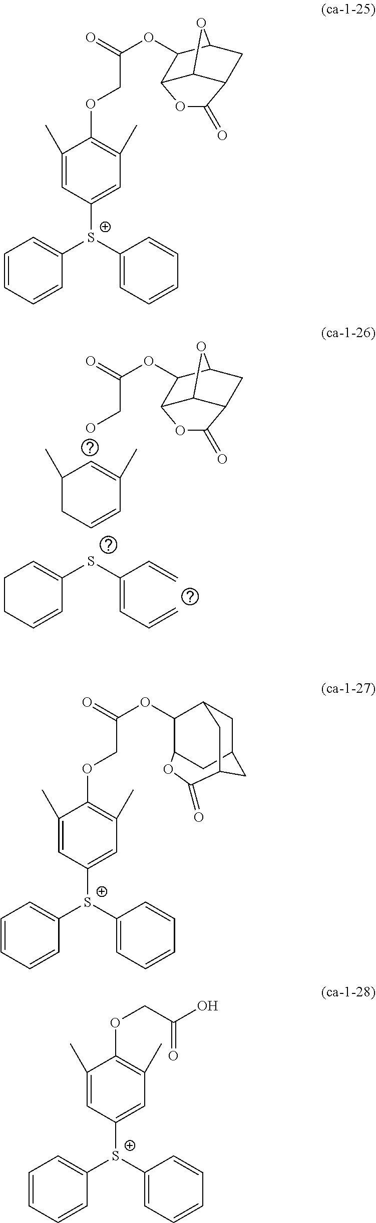

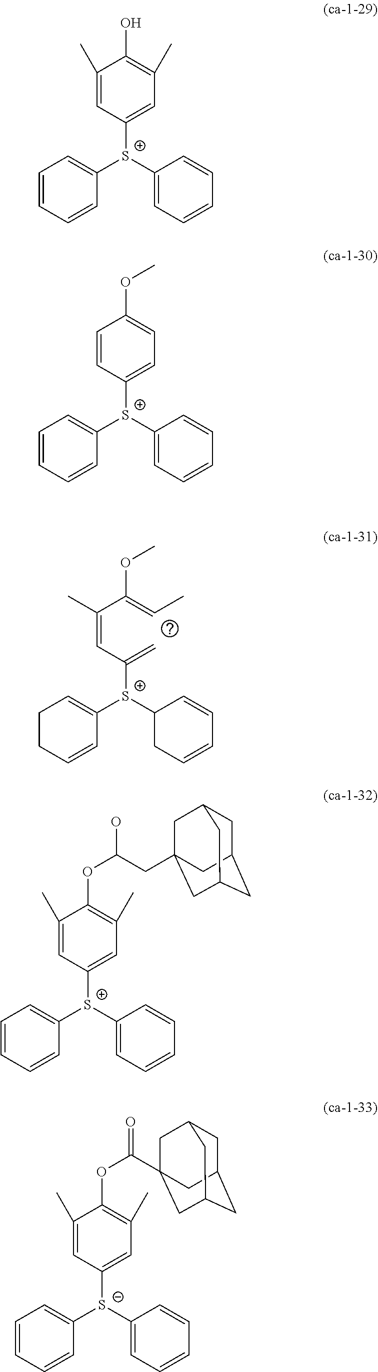 [SMILES: Cc1cc([S+](c2ccccc2)c2ccccc2)cc(C)c1O ; COc1ccc([S+](c2ccccc2)c2ccccc2)cc1 ; C=C(/C=C(C)\C(=C\C)OC)[S+](C1=CCCC=C1)C1C=CC=CC1 ; Cc1cc([S+](c2ccccc2)c2ccccc2)cc(C)c1OC(O)CC12CC3CC(CC(C3)C1)C2 ; Cc1cc([S-](c2ccccc2)c2ccccc2)cc(C)c1OC(=O)C12CC3CC(CC(C3)C1)C2]

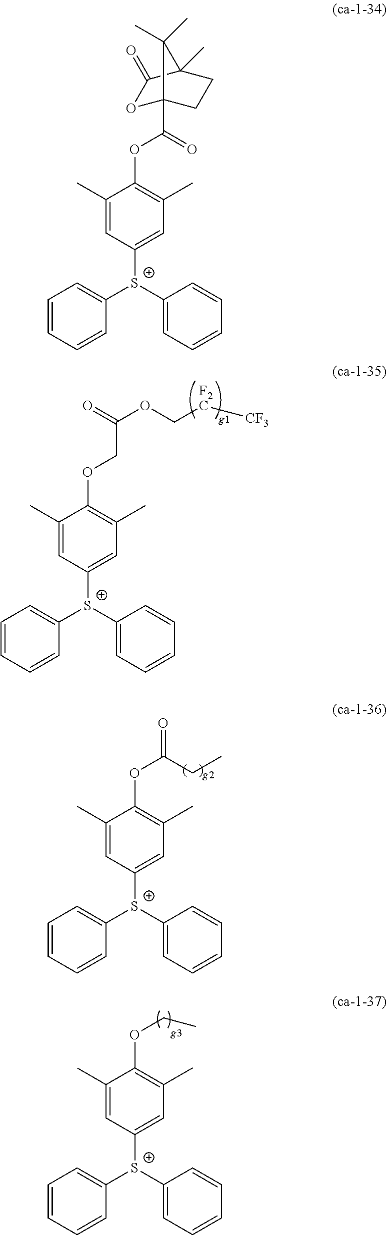

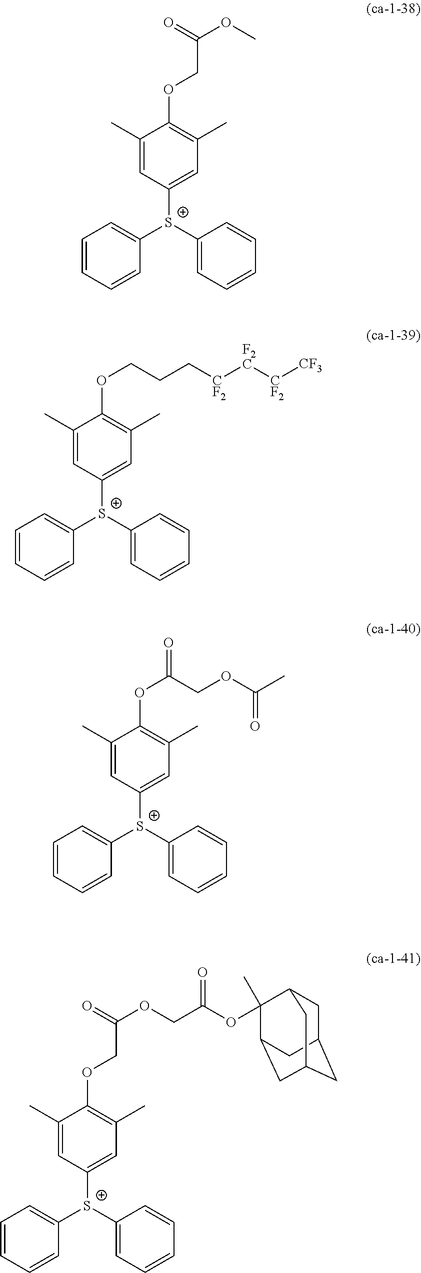 [SMILES: COC(=O)COc1c(C)cc([S+](c2ccccc2)c2ccccc2)cc1C ; Cc1cc([S+](c2ccccc2)c2ccccc2)cc(C)c1OCCCC(F)(F)C(F)(F)C(F)(F)C(F)(F)F ; CC(=O)OCC(=O)Oc1c(C)cc([S+](c2ccccc2)c2ccccc2)cc1C ; Cc1cc([S+](c2ccccc2)c2ccccc2)cc(C)c1OCC(=O)OCC(=O)OC1(C)C2CC3CC(C2)CC1C3]

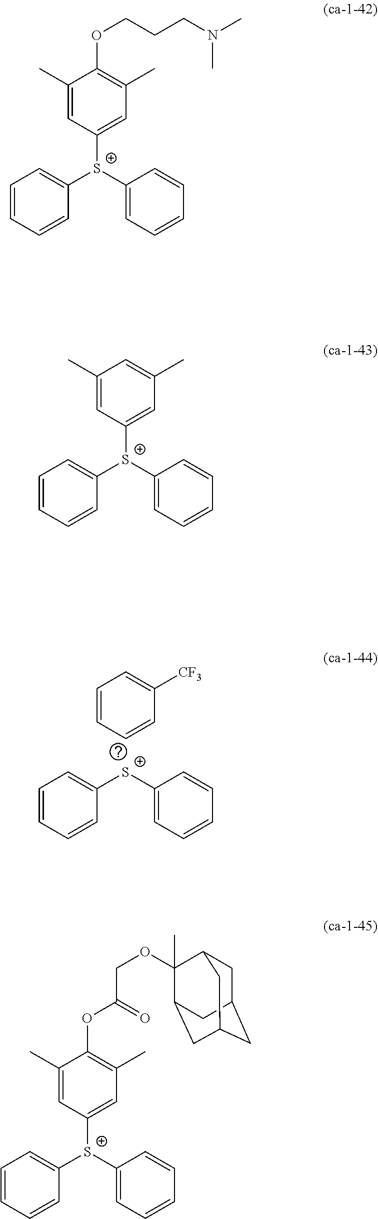

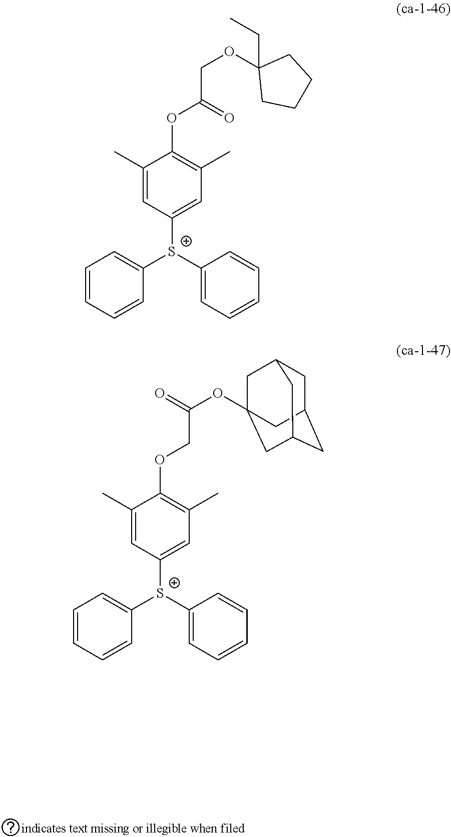 [SMILES: CCC1(OCC(=O)Oc2c(C)cc([S+](c3ccccc3)c3ccccc3)cc2C)CCCC1 ; Cc1cc([S+](c2ccccc2)c2ccccc2)cc(C)c1OCC(=O)OC12CC3CC(CC(C3)C1)C2]

[In the formulae, g1, g2, and g3 represent a repeating number, g1 represents an integer of 1 to 5, g2 represents an integer of 0 to 20, and g3 represents an integer of 0 to 20.]

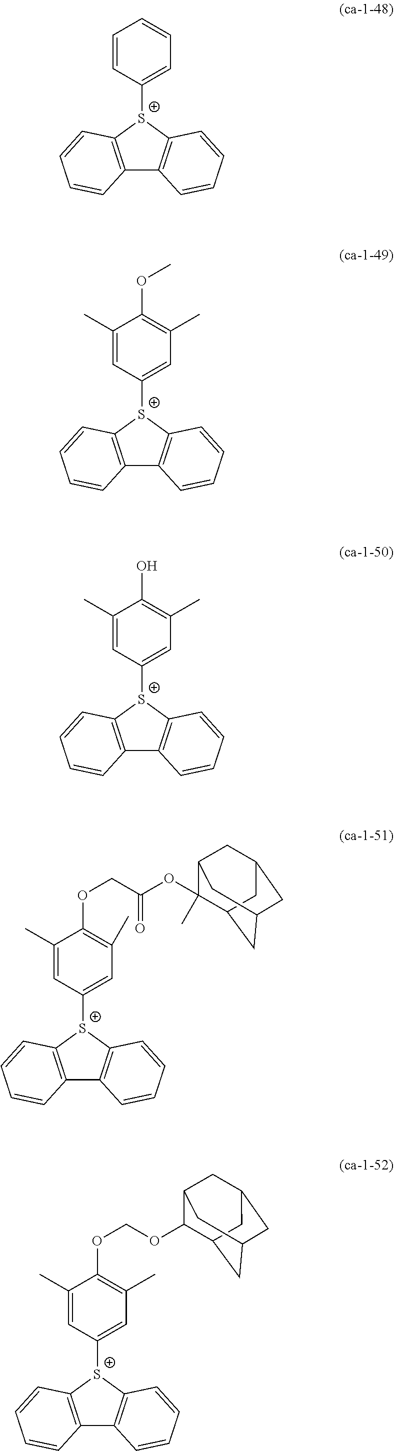 [SMILES: c1ccc(-[s+]2c3ccccc3c3ccccc32)cc1 ; COc1c(C)cc(-[s+]2c3ccccc3c3ccccc32)cc1C ; Cc1cc(-[s+]2c3ccccc3c3ccccc32)cc(C)c1O ; Cc1cc(-[s+]2c3ccccc3c3ccccc32)cc(C)c1OCC(=O)OC1(C)C2CC3CC(C2)CC1C3 ; Cc1cc(-[s+]2c3ccccc3c3ccccc32)cc(C)c1OCOC1C2CC3CC(C2)CC1C3]

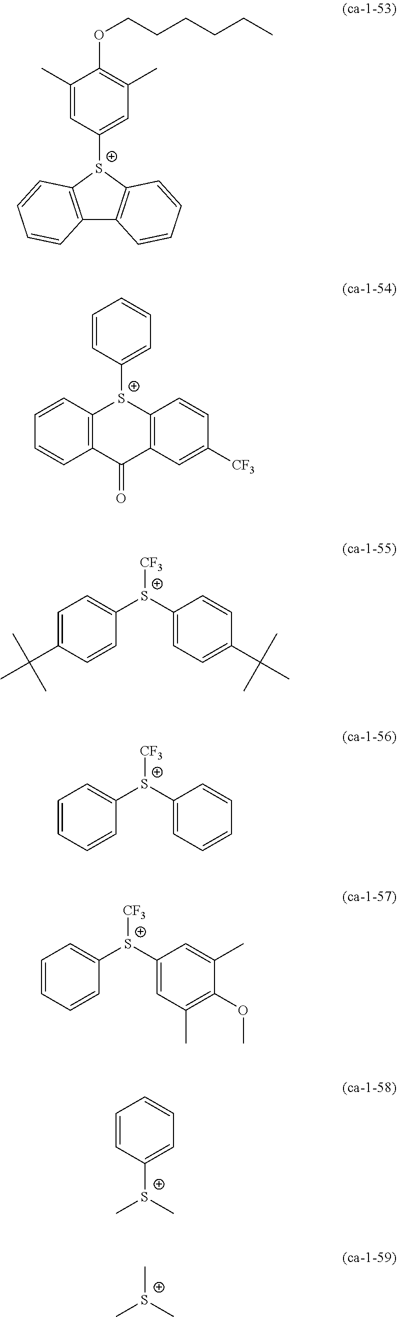

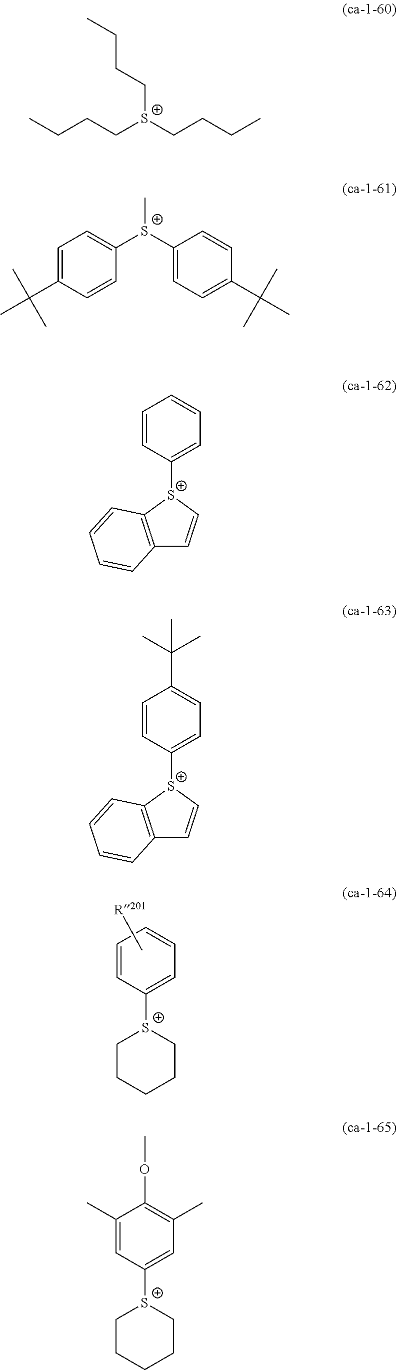

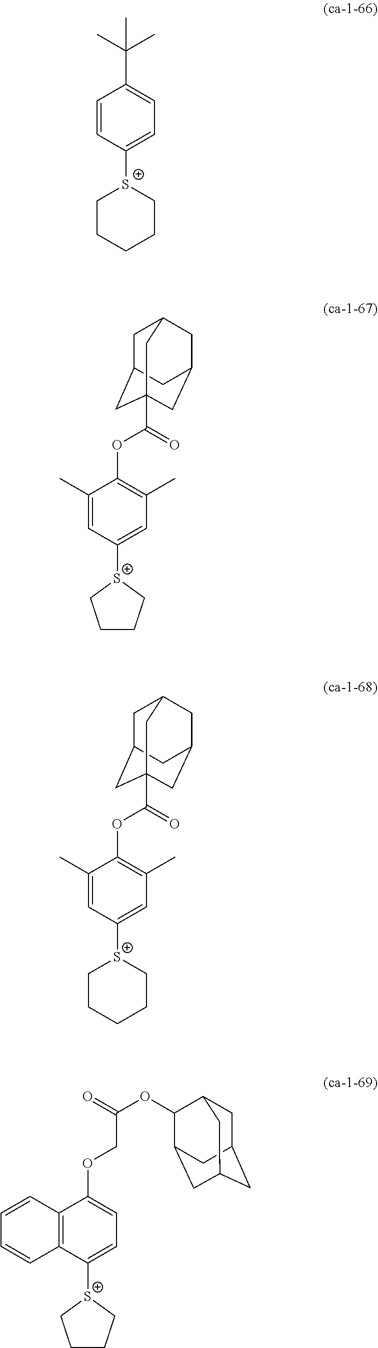 [SMILES: CC(C)(C)c1ccc([S+]2CCCCC2)cc1 ; Cc1cc([S+]2CCCC2)cc(C)c1OC(=O)C12CC3CC(CC(C3)C1)C2 ; Cc1cc([S+]2CCCCC2)cc(C)c1OC(=O)C12CC3CC(CC(C3)C1)C2 ; O=C(COc1ccc([S+]2CCCC2)c2ccccc12)OC1C2CC3CC(C2)CC1C3]

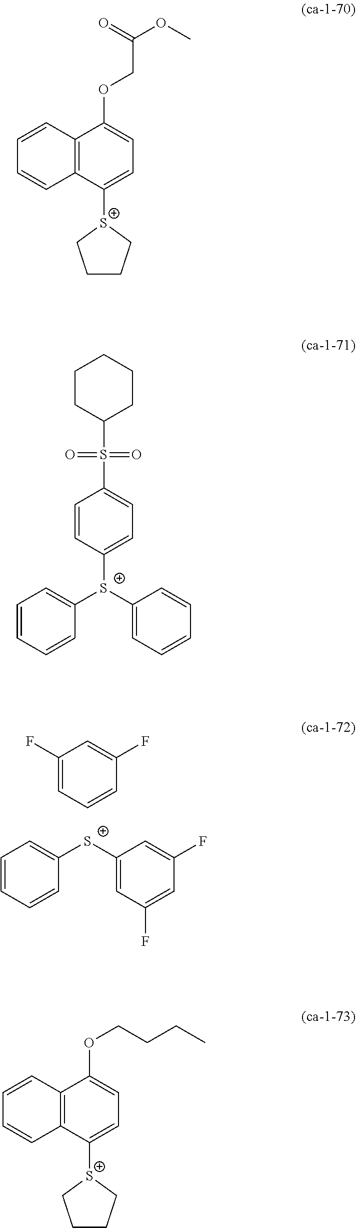

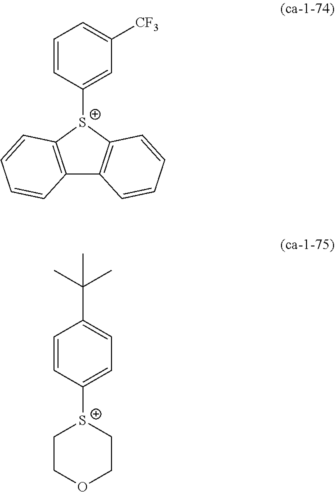 [SMILES: FC(F)(F)c1cccc(-[s+]2c3ccccc3c3ccccc32)c1 ; CC(C)(C)c1ccc([S+]2CCOCC2)cc1]

[In the formulae, R″²⁰¹ represents a hydrogen atom or a substituent, and examples of the substituent include the same groups as those for the substituents that R²⁰¹ to R²⁰⁷ and R²¹⁰ to R²¹² may have.]

Specific examples of suitable cations represented by Formula (ca-2) include a diphenyliodonium cation and a bis(4-tert-butylphenyl) iodonium cation.

Specific examples of suitable cations represented by Formula (ca-3) include cations each represented by Formulae (ca-3-1) to (ca-3-6).

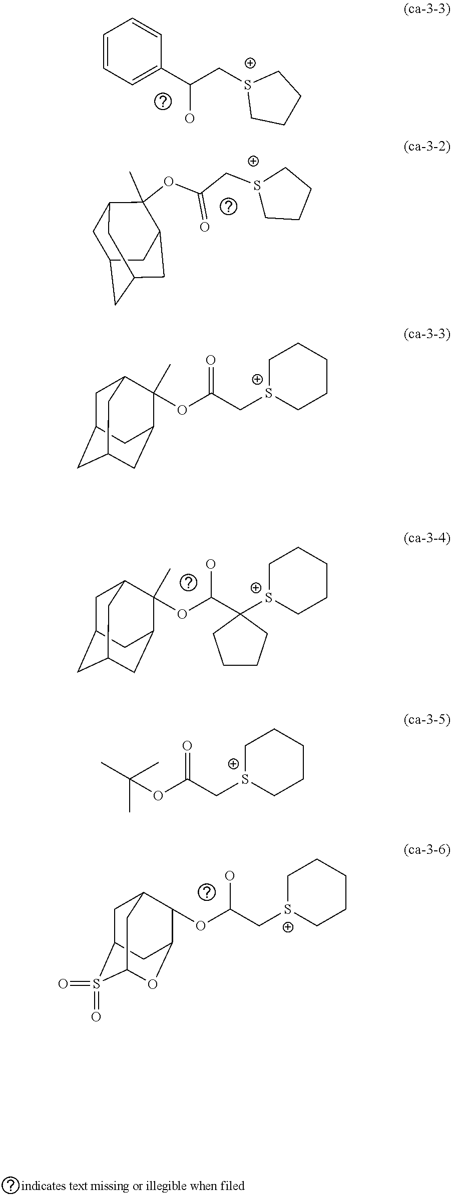

A cation represented by Formula (ca-1) is preferable as M^{m+}. Further, an m-valent cation having a fluorine atom is preferable as M^{m+}. A cation represented by Formula (ca-1-1) is preferable as M^{m+}.

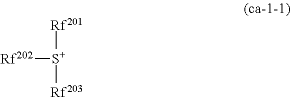

[In the formula, Rf²⁰¹ to Rf²⁰³ each independently represents an aryl group, an alkyl group, or an alkenyl group, which may have a substituent. Rf²⁰¹ to Rf²⁰³ may be bonded to each other to form a ring together with the sulfur atom in the formula. Here, at least one of Rf²⁰¹ to Rf²⁰³ includes at least one fluorine atom.]

Rf²⁰¹ to Rf²⁰³ in Formula (ca-1-1) each has the same definition as that for R²⁰¹ to R²⁰³ in Formula (ca-1). Here, at least one of Rf²⁰¹ to Rf²⁰³ includes at least one fluorine atom. The cation represented by Formula (ca-1-1) preferably contains three or more fluorine atoms. Any one of Rf²⁰¹ to Rf²⁰³ may have 3 or more fluorine atoms, and the total number of fluorine atoms included in Rf²⁰¹ to Rf²⁰³ may be 3 or more.

Specific examples of the constitutional unit (a0) are shown below, but the examples are not limited thereto. In the formulae shown below, R^{α} represents a hydrogen atom, a methyl group, or a trifluoromethyl group.

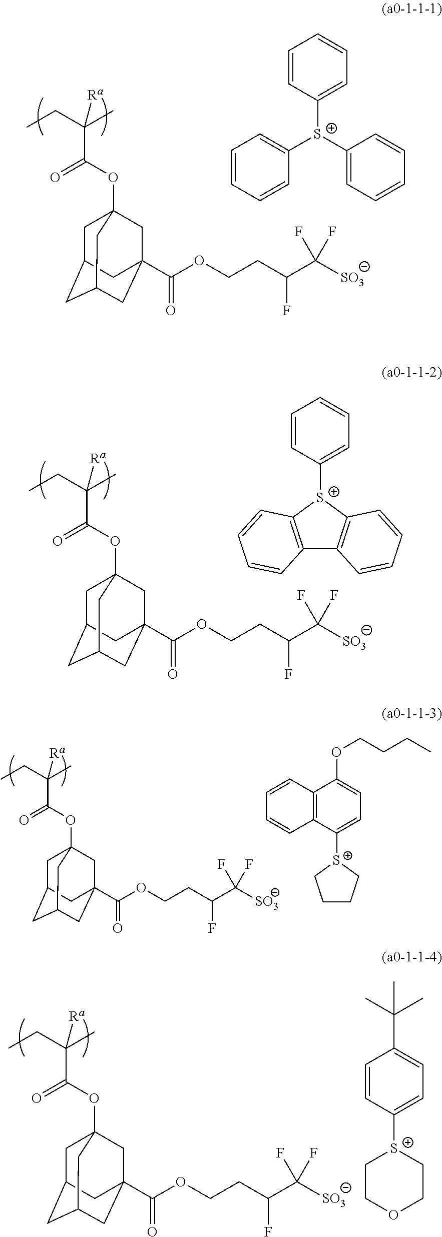

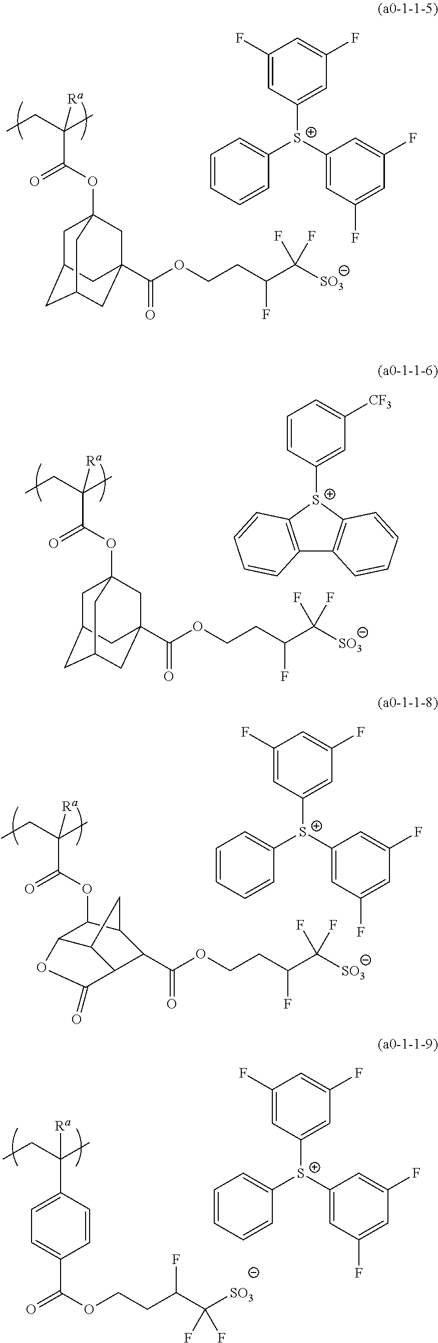

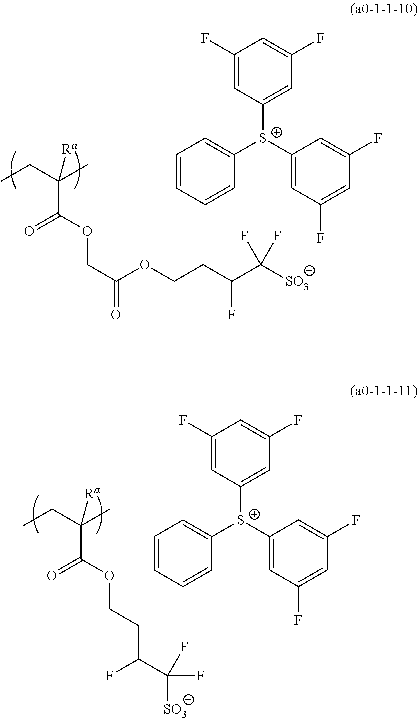

The constitutional unit (a0) contained in the component (A1) may be used alone or two or more kinds thereof may be used.

The proportion of the constitutional unit (a0) in the component (A1) is preferably in a range of 1% to 50% by mole, more preferably in a range of 2% to 35% by mole, still more preferably in a range of 5% to 30% by mole, and particularly preferably in a range of 10% to 25% by mole with respect to the total amount (100% by mole) of all constitutional units constituting the component (A1).

In a case where the proportion of the constitutional unit (a0) is greater than or equal to the lower limits of the above-described preferable ranges, the sensitivity is further improved. In a case where the proportion of the constitutional unit (a0) is less than or equal to the upper limits of the above-described preferable ranges, the pattern shape is further improved, and the roughness and defects (such as coating defects) are further reduced.

### <<Other Constitutional Units>>

The component (A1) may have other constitutional units in addition to the above-described constitutional unit (a0).

Examples of the other constitutional units include a constitutional unit (a1) containing an acid-decomposable group whose polarity is increased due to the action of an acid; a constitutional unit (a10) represented by General Formula (a10-1); a constitutional unit (a2) containing a lactone-containing cyclic group; and a constitutional unit (a8) derived from a compound represented by General Formula (a8-1).

### Constitutional Unit (a1):

The constitutional unit (a1) is a constitutional unit that contains an acid-decomposable group whose polarity is increased by the action of an acid.

Examples of the acid-dissociable group are the same as those which have been suggested as the acid-dissociable groups of the base resin for a chemically amplified resist composition.

Specific examples of the suggested acid-dissociable group of the base resin for a chemically amplified resist composition include an “acetal type acid-dissociable group”, a “tertiary alkyl ester type acid-dissociable group”, a “tertiary alkyloxycarbonyl acid-dissociable group”, and a “secondary alkyloxycarbonyl acid-dissociable group” described below.

### Acetal Type Acid-Dissociable Group:

Examples of the acid-dissociable group that protects a carboxy group or a hydroxyl group in the polar groups include an acid-dissociable group represented by General Formula (a1-r-1) (hereinafter, also referred to as “acetal type acid-dissociable group”).

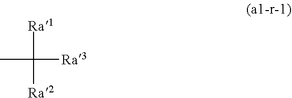

[In the formula, Ra′¹ and Ra′² represent a hydrogen atom or an alkyl group. Ra′³ represents a hydrocarbon group, and Ra′³ may be bonded to any of Ra′¹ and Ra′² to form a ring.]

In Formula (a1-r-1), it is preferable that at least one of Ra′¹ or Ra′² represent a hydrogen atom and more preferable that both Ra′¹ and Ra′² represent a hydrogen atom.

In a case where Ra′¹ or Ra′² represents an alkyl group, examples of the alkyl group include the same as those exemplified as the substituent which may be bonded to the carbon atom at the α-position in the description of the α-substituted acrylic acid ester. Among these, an alkyl group having 1 to 5 carbon atoms is preferable. Specific preferred examples thereof include linear or branched alkyl groups. More specific examples thereof include a methyl group, an ethyl group, a propyl group, an isopropyl group, an n-butyl group, an isobutyl group, a tert-butyl group, a pentyl group, an isopentyl group, and a neopentyl group. Among these, a methyl group or an ethyl group is more preferable, and a methyl group is particularly preferable.

In Formula (a1-r-1), examples of the hydrocarbon group as Ra′³ include a linear or branched alkyl group and a cyclic hydrocarbon group.

The linear alkyl group has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group has preferably 3 to 10 carbon atoms and more preferably 3 to 5 carbon atoms. Specific examples thereof include an isopropyl group, an isobutyl group, a tert-butyl group, an isopentyl group, a neopentyl group a 1,1-diethylpropyl group, and a 2,2-dimethylbutyl group. Among these, an isopropyl group is preferable.

In a case where Ra′³ represents a cyclic hydrocarbon group, the hydrocarbon group may be an aliphatic hydrocarbon group or an aromatic hydrocarbon group and may be a polycyclic group or a monocyclic group.

As the aliphatic hydrocarbon group which is a monocyclic group, a group in which one hydrogen atom has been removed from a monocycloalkane is preferable. The monocycloalkane has preferably 3 to 6 carbon atoms, and specific examples thereof include cyclopentane and cyclohexane.

As the aliphatic hydrocarbon group which is a polycyclic group, a group in which one hydrogen atom has been removed from a polycycloalkane is preferable. As the polycycloalkane, a group having 7 to 12 carbon atoms is preferable, and specific examples thereof include adamantane, norbornane, isobornane, tricyclodecane, and tetracyclododecane.

In a case where the cyclic hydrocarbon group as Ra′³ is an aromatic hydrocarbon group, the aromatic hydrocarbon group is a hydrocarbon group having at least one aromatic ring.

The aromatic ring is not particularly limited as long as it is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms.

Specific examples of the aromatic ring include aromatic hydrocarbon rings such as benzene, naphthalene, anthracene, and phenanthrene; and aromatic heterocyclic rings in which some carbon atoms constituting the above-described aromatic hydrocarbon rings have been substituted with heteroatoms. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group as Ra′³ include a group in which one hydrogen atom has been removed from the above-described aromatic hydrocarbon ring or aromatic heterocyclic ring (such as an aryl group or a heteroaryl group); a group in which one hydrogen atom has been removed from an aromatic compound having two or more aromatic rings (such as biphenyl or fluorene); and a group in which one hydrogen atom of the above-described aromatic hydrocarbon ring or aromatic heterocyclic ring has been substituted with an alkylene group (for example, an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aromatic hydrocarbon ring or aromatic heterocyclic ring is preferably in a range of 1 to 4, more preferably 1 or 2, and particularly preferably 1.

The cyclic hydrocarbon group as Ra′³ may include a substituent. Examples of the substituent include Ra^{x5} described above.

In a case where Ra′³ is bonded to any of Ra′¹ and Ra′² to form a ring, the cyclic group is preferably a 4- to 7-membered ring and more preferably a 4- to 6-membered ring. Specific examples of the cyclic group include a tetrahydropyranyl group and a tetrahydrofuranyl group.

Tertiary alkyl ester type acid-dissociable group:

Examples of the acid-dissociable group that protects a carboxy group among the polar groups include an acid-dissociable group represented by General Formula (a1-r-2).

Among examples of the acid-dissociable group represented by Formula (a1-r-2), a group formed of an alkyl group is referred to as “tertiary alkyl ester type acid-dissociable group” for convenience.

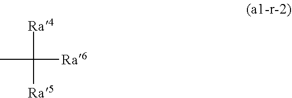

[In the formula, Ra′⁴ to Ra′⁶ each represents a hydrocarbon group, and Ra′⁵ and Ra′⁶ may be bonded to each other to form a ring.]

Examples of the hydrocarbon group as Ra′⁴ include a linear or branched alkyl group, a chain-like or cyclic alkenyl group, and a cyclic hydrocarbon group.

Examples of the linear or branched alkyl group and the cyclic hydrocarbon group (an aliphatic hydrocarbon group which is a monocyclic group, an aliphatic hydrocarbon group which is a polycyclic group, or an aromatic hydrocarbon group) as Ra′⁴ include the same groups as those for Ra′³.

As the chain-like or cyclic alkenyl group as Ra′⁴, an alkenyl group having 2 to carbon atoms is preferable.

Examples of the hydrocarbon group as Ra′⁵ or Ra′⁶ include the same groups as those for Ra′³.

In a case where Ra′⁵ and Ra′⁶ are bonded to each other to form a ring, suitable examples thereof include a group represented by General Formula (a1-r2-1), a group represented by General Formula (a1-r2-2), and a group represented by General Formula (a1-r2-3).

Meanwhile, in a case where Ra′⁴ to Ra′⁶ independently represent a hydrocarbon group without being bonded to one another, suitable examples thereof include a group represented by General Formula (a1-r2-4).

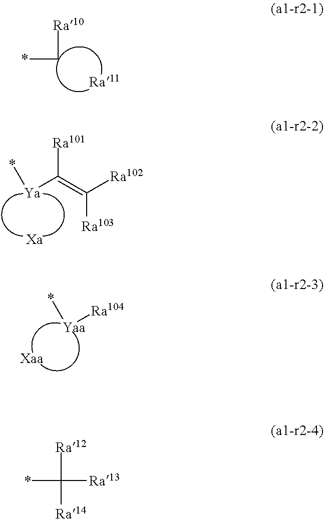

[In Formula (a1-r2-1), Ra′¹⁰ represents a linear or branched alkyl group having 1 to 12 carbon atoms, in which a part thereof may be substituted with a halogen atom or a heteroatom-containing group. Ra′¹¹ represents a group that forms an aliphatic cyclic group with the carbon atom to which Ra′¹⁰ has been bonded. In Formula (a1-r2-2), Ya represents a carbon atom. Xa represents a group that forms a cyclic hydrocarbon group with Ya. Some or all hydrogen atoms in this cyclic hydrocarbon group may be substituted. Ra¹⁰¹ to Ra¹⁰³ (23 each independently represents a hydrogen atom, a chain-like monovalent saturated hydrocarbon group having 1 to 10 carbon atoms, or a monovalent aliphatic cyclic saturated hydrocarbon group having 3 to 20 carbon atoms. Some or all hydrogen atoms in the chain-like saturated hydrocarbon group and the aliphatic cyclic saturated hydrocarbon group may be substituted. Two or more of Ra¹⁰¹ to Ra¹⁰³ may be bonded to each other to form a cyclic structure. In Formula (a1-r2-3), Yaa represents a carbon atom. Xaa represents a group that forms an aliphatic cyclic group with Yaa. Ra¹⁰⁴ represents an aromatic hydrocarbon group which may have a substituent. In Formula (a1-r2-4), Ra′¹² and Ra′¹³ each independently represents a chain-like monovalent saturated hydrocarbon group having 1 to 10 carbon atoms. Some or all hydrogen atoms in this chain-like saturated hydrocarbon group may be substituted. Ra′¹⁴ represents a hydrocarbon group which may have a substituent. * represents a bonding site (the same applies hereinafter).]

In Formula (a1-r2-1), Ra′¹⁰ represents a linear or branched alkyl group having 1 to 12 carbon atoms, in which a part thereof may be substituted with a halogen atom or a heteroatom-containing group.

The linear alkyl group as Ra′¹⁰ has 1 to 12 carbon atoms, preferably 1 to 10 carbon atoms, and particularly preferably 1 to 5 carbon atoms.

Examples of the branched alkyl group as Ra′¹⁰ include those for Ra′³ described above.

The alkyl group in Ra′¹⁰ may be partially substituted with a halogen atom or a heteroatom-containing group. For example, some hydrogen atoms constituting the alkyl group may be substituted with a halogen atom or a heteroatom-containing group. Further, some carbon atoms (methylene group or the like) constituting the alkyl group may be substituted with a heteroatom-containing group.

Examples of the heteroatoms here include an oxygen atom, a nitrogen atom, and a sulfur atom. Examples of the heteroatom-containing group include (—O—), —C(═O)—O—, —O—C(═O))—, —C(═O)—, —O—C(═O)—O)—, —C(═O)—NH—, —NH—, —S—, —S(═O)₂—, and —S(═O)₂—O—.

In Formula (a1-r2-1), preferred examples of Ra′¹¹ (an aliphatic cyclic group that is formed together with a carbon atom to which Ra′¹⁰ is bonded) include the groups exemplified as the aliphatic hydrocarbon group (alicyclic hydrocarbon group) which is a monocyclic group or a polycyclic group as Ra′³ in Formula (a1-r-1). Among them, it is preferably a monocyclic alicyclic hydrocarbon group, and specifically, it is more preferably a cyclopentyl group or a cyclohexyl group.

In Formula (a1-r2-2), examples of the cyclic hydrocarbon group that is formed by Xa together with Ya include a group in which one or more hydrogen atoms have been further removed from the cyclic monovalent hydrocarbon group (aliphatic hydrocarbon group) as Ra′³ in Formula (a1-f-1).

The cyclic hydrocarbon group that is formed by Xa together with Ya may have a substituent. Examples of the substituent include those exemplified as the substituents that the cyclic hydrocarbon group as Ra′³ may have.

In Formula (a1-r2-2), examples of the chain-like monovalent saturated hydrocarbon group having 1 to 10 carbon atoms as Ra¹⁰¹ to Ra³⁰³ include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms as Ra¹⁰¹ to Ra¹⁰³ include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2]octanyl group, a tricyclo[5.2.1.02,6]decanyl group, a tricyclo[3.3.1.13,7]decanyl group, a tetracyclo[6.2.1.13,6.02,7]dodecanyl group, or an adamantyl group.

From the viewpoint of ease of synthesis, Ra¹⁰¹ to Ra¹⁰³ represent preferably a hydrogen atom or a chain-like monovalent saturated hydrocarbon group having 1 to 10 carbon atoms, more preferably a hydrogen atom, a methyl group, or an ethyl group, and particularly preferably a hydrogen atom.

Examples of the substituent included in the chain-like saturated hydrocarbon group or the aliphatic cyclic saturated hydrocarbon group represented by Ra¹⁰¹ to Ra¹⁰³ include the same substituents as those for Ra^{x5}.

Examples of the group having a carbon-carbon double bond generated by two or more of Ra¹⁰¹ to Ra¹⁰³ being bonded to each other to form a cyclic structure include a cyclopentenyl group, a cyclohexenyl group, a methylcyclopentenyl group, a methylcyclohexenyl group, a cyclopentylidenethenyl group, and a cyclohexylidenethenyl group. Among these, from the viewpoint of ease of synthesis, a cyclopentenyl group, a cyclohexenyl group, or a cyclopentylidenethenyl group is preferable.

In Formula (a1-r2-3), as the aliphatic cyclic group that is formed by Xaa together with Yaa, the group exemplified as the aliphatic hydrocarbon group which is a monocyclic group or a polycyclic group as Ra′³ in Formula (a1-r-1) is preferable.

In Formula (a1-r2-3), examples of the aromatic hydrocarbon group as Ra¹⁰⁴ include a group in which one or more hydrogen atoms have been removed from an aromatic hydrocarbon ring having 5 to 30 carbon atoms. Among the examples, Ra¹⁰⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from benzene or naphthalene, and most preferably a group in which one or more hydrogen atoms have been removed from benzene.

Examples of the substituent that Ra¹⁰⁴ in Formula (a1-r2-3) may have include a methyl group, an ethyl group, a propyl group, a hydroxy group, a carboxy group, a halogen atom, an alkoxy group (such as a methoxy group, an ethoxy group, a propoxy group, or a butoxy group), and an alkyloxycarbonyl group.

In Formula (a1-r2-4), Ra′¹² and Ra′¹³ each independently represents a chain-like monovalent saturated hydrocarbon group having 1 to 10 carbon atoms. Examples of the chain-like monovalent saturated hydrocarbon group having 1 to 10 carbon atoms as Ra′¹² and Ra′¹³ include those exemplified as the chain-like monovalent saturated hydrocarbon group having 1 to 10 carbon atoms as Ra¹⁰¹ to Ra¹⁰³. Some or all hydrogen atoms in this chain-like saturated hydrocarbon group may be substituted.

Ra′¹² and Ra′¹³ represent preferably an alkyl group having 1 to 5 carbon atoms, more preferably an alkyl group having 1 to 5 carbon atoms, still more preferably a methyl group or an ethyl group, and particularly preferably a methyl group.

In a case where the chain-like saturated hydrocarbon group represented by Ra′¹² and Ra′¹³ is substituted, examples of the substituent thereof include the same substituents as those for Ra^{x5}.

In Formula (a1-r2-4), Ra′¹⁴ represents a hydrocarbon group which may have a substituent. Examples of the hydrocarbon group as Ra′¹⁴ include a linear or branched alkyl group and a cyclic hydrocarbon group.

The linear alkyl group as Ra′¹⁴ has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group as Ra′¹⁴ has preferably 3 to 10 carbon atoms and more preferably 3 to 5 carbon atoms. Specific examples thereof include an isopropyl group, an isobutyl group, a tert-butyl group, an isopentyl group, a neopentyl group a 1,1-diethylpropyl group, and a 2,2-dimethylbutyl group. Among these, an isopropyl group is preferable.

In a case where Ra′¹⁴ represents a cyclic hydrocarbon group, the hydrocarbon group may be an aliphatic hydrocarbon group or an aromatic hydrocarbon group and may be a polycyclic group or a monocyclic group.

As the aliphatic hydrocarbon group which is a monocyclic group, a group in which one hydrogen atom has been removed from a monocycloalkane is preferable. The monocycloalkane has preferably 3 to 6 carbon atoms, and specific examples thereof include cyclopentane and cyclohexane.

As the aliphatic hydrocarbon group which is a polycyclic group, a group in which one hydrogen atom has been removed from a polycycloalkane is preferable. As the polycycloalkane, a group having 7 to 12 carbon atoms is preferable, and specific examples thereof include adamantane, norbornane, isobornane, tricyclodecane, and tetracyclododecane.

Examples of the aromatic hydrocarbon group as Ra′¹⁴ include the same groups as those for the aromatic hydrocarbon group as Ra¹⁰⁴. Among these, Ra′¹⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from naphthalene or anthracene, and most preferably a group in which one or more hydrogen atoms have been removed from naphthalene.

Examples of the substituent that Ra′¹⁴ may have include the same groups as those for the substituent that Ra¹⁰⁴ may have.

In a case where Ra′¹⁴ in Formula (a1-r2-4) represents a naphthyl group, the position bonded to the tertiary carbon atom in Formula (a1-r2-4) may be the 1-position or the 2-position of the naphthyl group.

In a case where Ra′¹⁴ in Formula (a1-r2-4) represents an anthryl group, the position bonded to the tertiary carbon atom in Formula (a1-r2-4) may be the 1-position, the 2-position, or the 9-position of the anthryl group.

Specific examples of the group represented by Formula (a1-r2-1) are shown below.

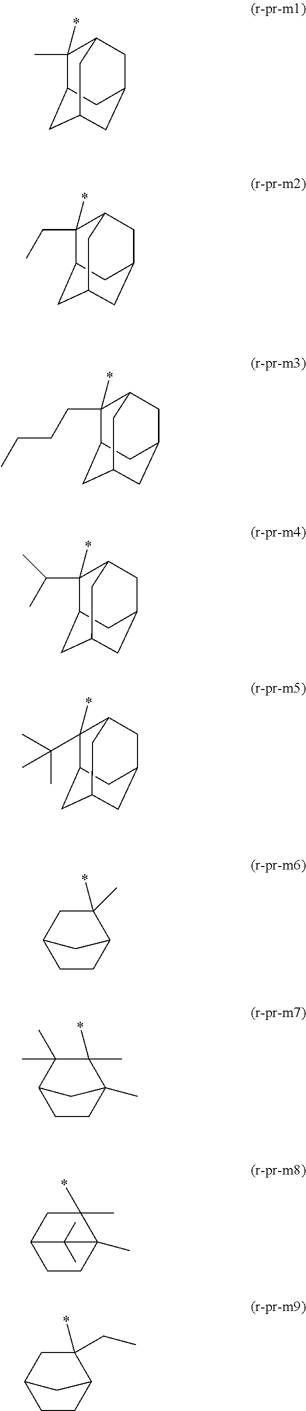

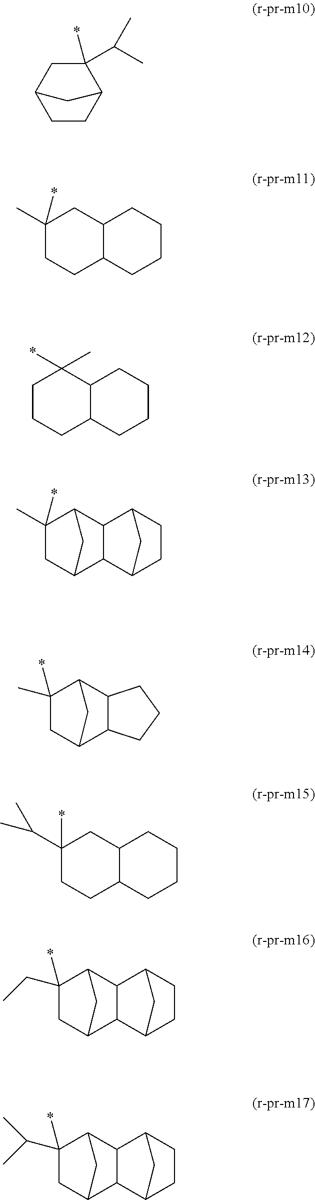

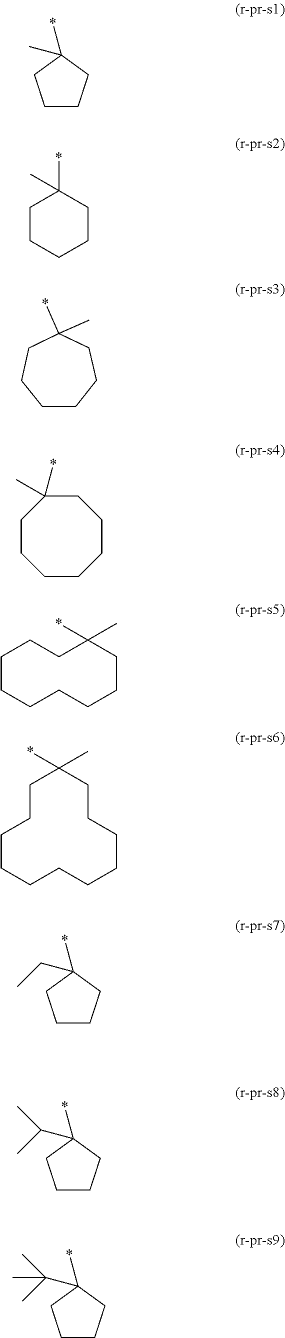

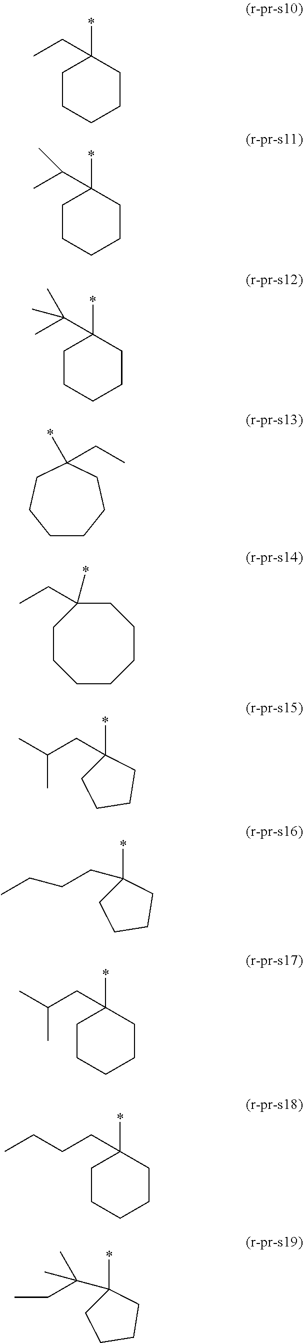

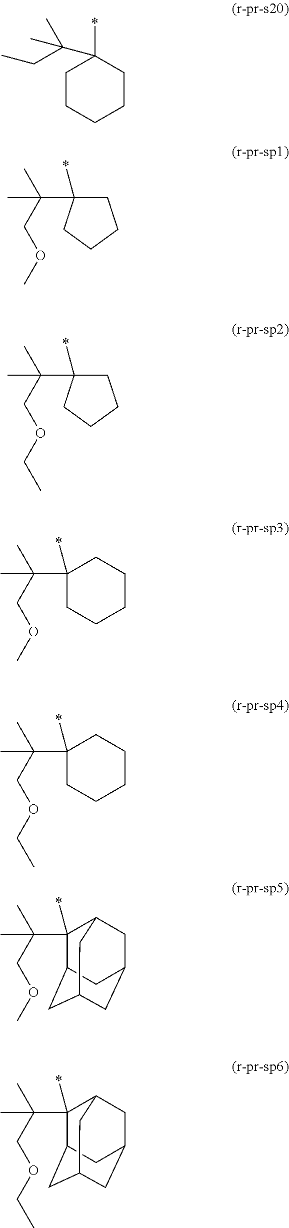

Specific examples of the group represented by Formula (a1-r2-2) are shown below.

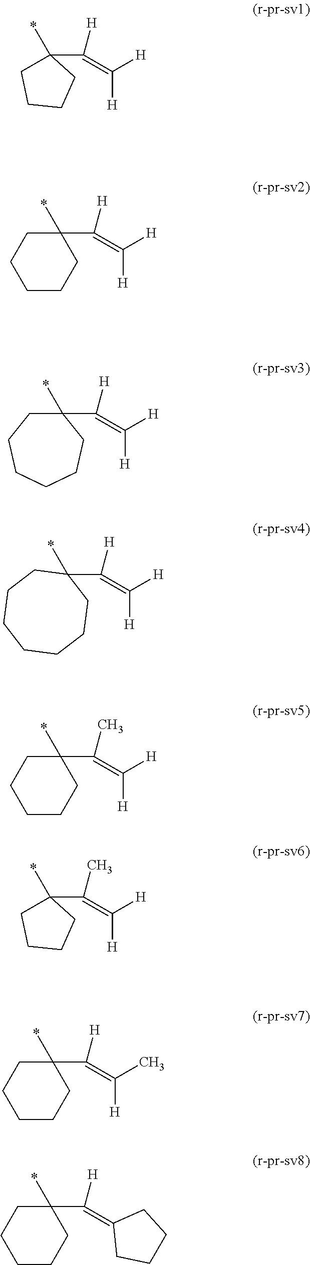

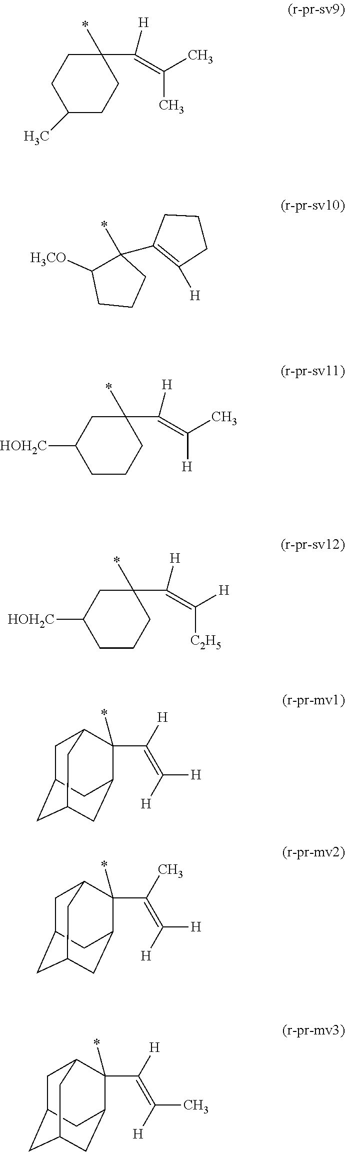

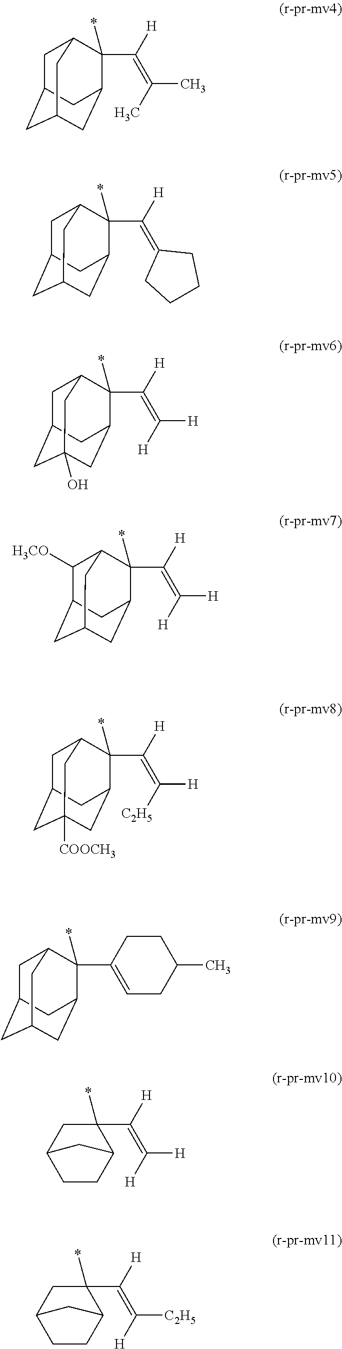

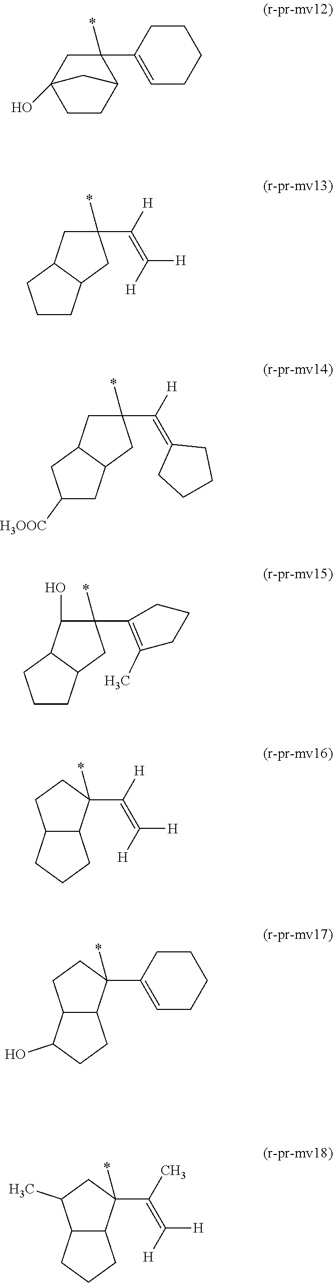

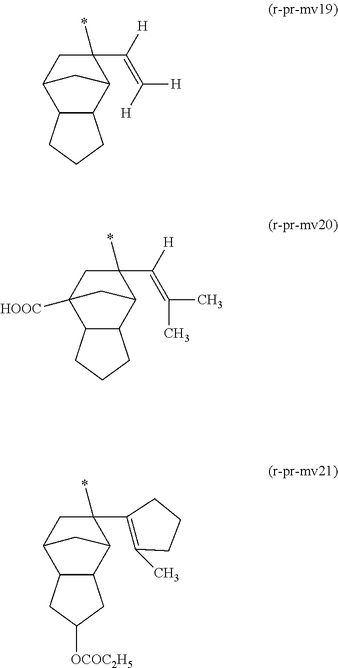

Specific examples of the group represented by Formula (a1-r2-3) are shown below.

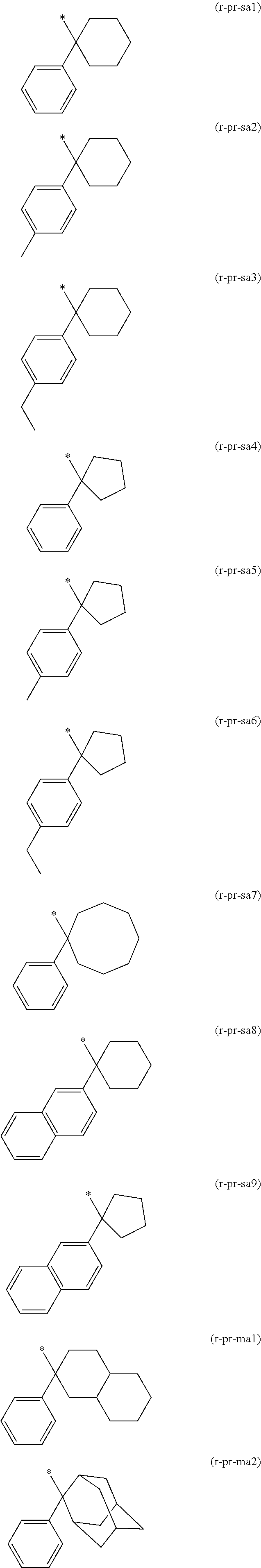

Specific examples of the group represented by Formula (a1-r2-4) are shown below.

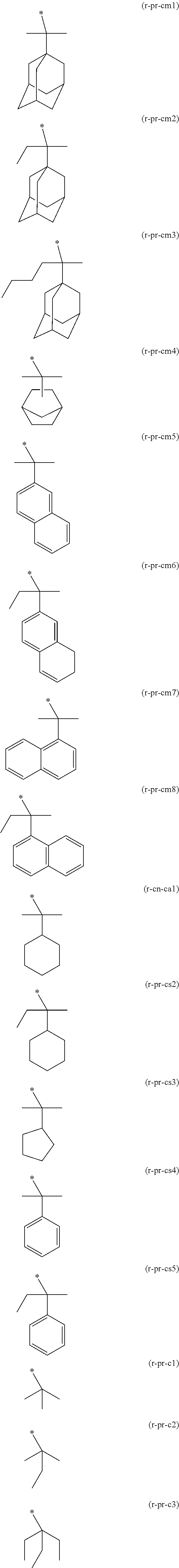

### Tertiary Alkyloxycarbonyl Acid-Dissociable Group:

Examples of the acid-dissociable group that protects a hydroxyl group among the polar groups include an acid-dissociable group (hereinafter, also referred to as “tertiary alkyloxycarbonyl acid-dissociable group” for convenience) represented by General Formula (a1-r-3).

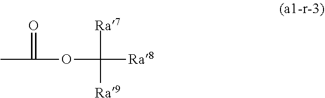

[In the formula, Ra′⁷ to Ra′⁹ each represents an alkyl group.]

In Formula (a1-r-3), Ra′⁷ to Ra′⁹ each represent preferably an alkyl group having 1 to 5 carbon atoms and more preferably an alkyl group having 1 to 3 carbon atoms.

Further, the total number of carbon atoms in each alkyl group is preferably in a range of 3 to 7, more preferably in a range of 3 to 5, and most preferably 3 or 4.

### Secondary Alkyl Ester Type Acid-Dissociable Group:

Examples of the acid-dissociable group that protects a carboxy group among the polar groups include an acid-dissociable group represented by General Formula (a1-r-4).

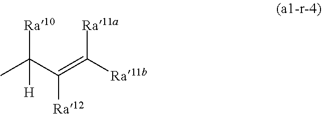

[In the formula, Ra′¹⁰ represents a hydrocarbon group. Ra′¹¹a and Ra′^{11b} each independently represents a hydrogen atom, a halogen atom, or an alkyl group. Ra′¹² represents a hydrogen atom or a hydrocarbon group. Ra′¹⁰ and Ra′^{11a} or Ra′^{11b} may be bonded to each other to form a ring. Ra′^{11a} or Ra′^{11b} and Ra′¹² may be bonded to each other to form a ring.]

Examples of the hydrocarbon group as Ra′¹⁰ or Ra′¹² in the formula include the same groups as those for Ra′³.

Examples of the alkyl group as Ra′^{11a} and Ra′^{11b} in the formula include the same groups as those for the alkyl group as Ra′¹.

In the formula, the hydrocarbon group as Ra′¹⁰ or Ra′¹² and the alkyl group as Ra′^{11a} and Ra′^{11b} may have a substituent. Examples of the substituent include Ra^{x5} described above.

Ra′¹⁰ and Ra′^{11a} or Ra′^{11b} may be bonded to each other to form a ring. The ring may be a polycyclic ring or a monocyclic ring, and may be an alicyclic ring or an aromatic ring.

The alicyclic ring and the aromatic ring may have a heteroatom.

Among the examples described above, as the ring formed by Ra′¹⁰ and Ra′^{11a} or Ra′^{11b} being bonded to each other, monocycloalkene, a ring in which some carbon atoms of monocycloalkene are substituted with heteroatoms (such as an oxygen atom and a sulfur atom), or monocycloalkadiene is preferable, cycloalkene having 3 to 6 carbon atoms is preferable, and cyclopentene or cyclohexene is preferable.

The ring formed by Ra′¹⁰ and Ra′^{11a} or Ra′^{11b} being bonded to each other may be a condensed ring. Specific examples of the condensed ring include indane.

The ring formed by Ra′¹⁰ and Ra′^{11a} or Ra′^{11b} being bonded to each other may have a substituent. Examples of the substituent include Ra^{x5} described above.

Ra′^{11a} or Ra′^{11b} and Ra′¹² may be bonded to each other to form a ring, and examples of the ring include the rings formed by Ra′¹⁰ and Ra′^{11a} or Ra′^{11b} being bonded to each other.

Specific examples of the group represented by Formula (a1-r-4) are shown below.

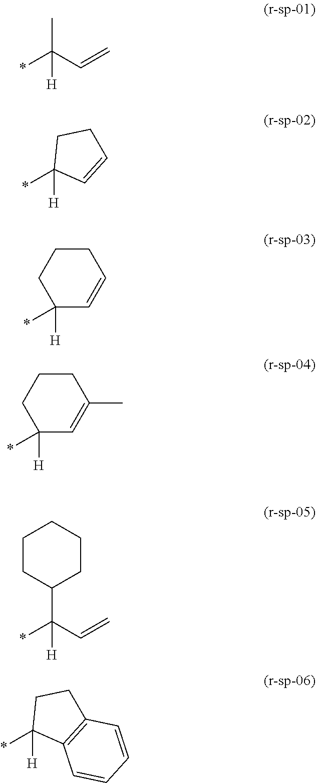

Examples of the constitutional unit (a1) include a constitutional unit derived from acrylic acid ester in which the hydrogen atom bonded to the carbon atom at the α-position may be substituted with a substituent; a constitutional unit derived from acrylamide; a constitutional unit in which at least some hydrogen atoms in a hydroxyl group of a constitutional unit derived from hydroxystyrene or a hydroxystyrene derivative are protected by a substituent containing the acid-decomposable group; and a constitutional unit in which at least some hydrogen atoms in —C(═O)—OH of a constitutional unit derived from vinylbenzoic acid or a vinylbenzoic acid derivative are protected by a substituent containing the acid-decomposable group.

Specific examples of the constitutional unit (a1) are shown below. In the formulae shown below, R^{α} represents a hydrogen atom, a methyl group, or a trifluoromethyl group.

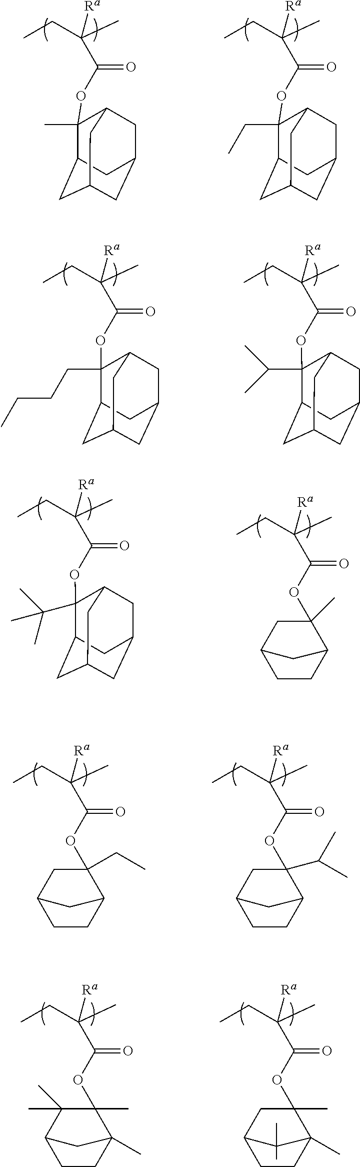

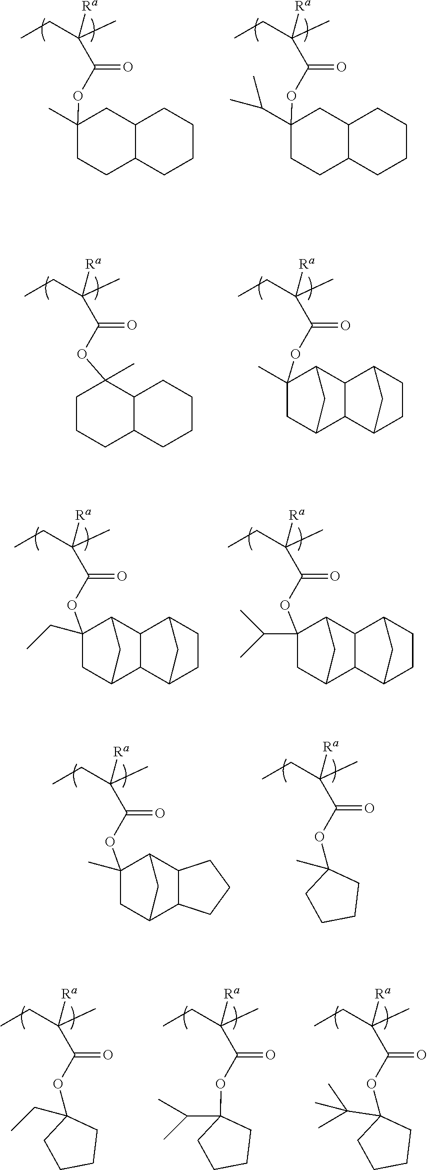

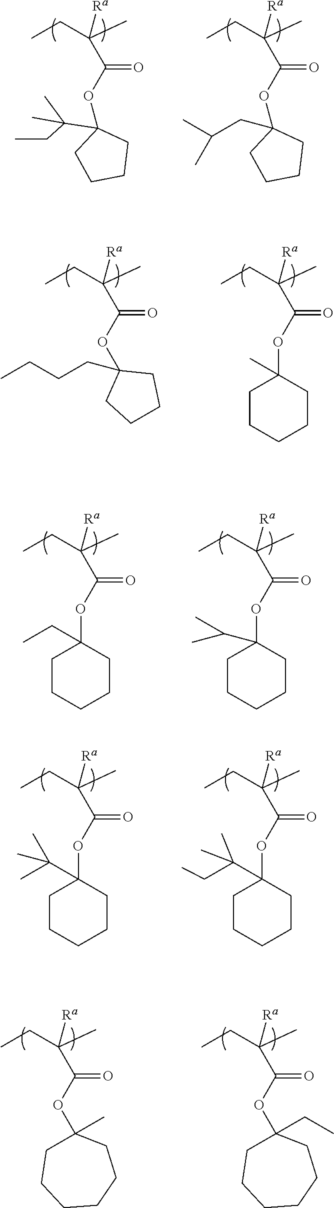

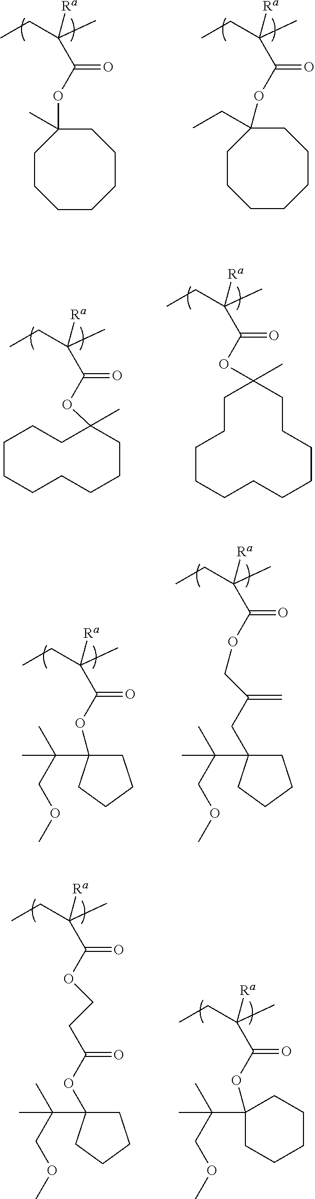

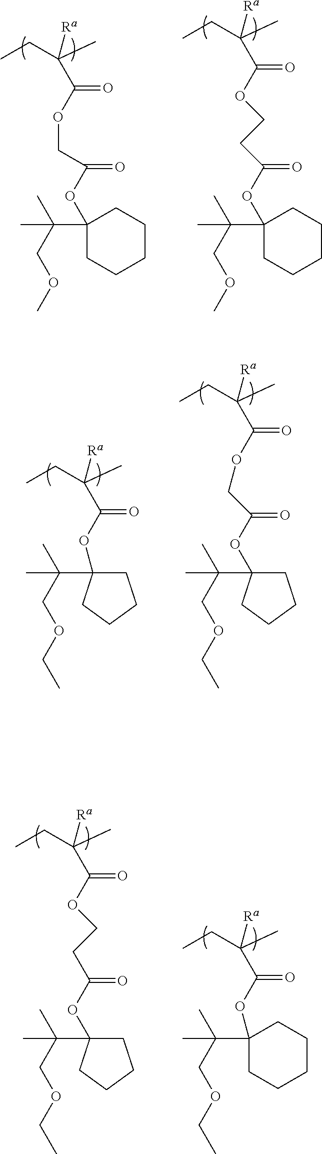

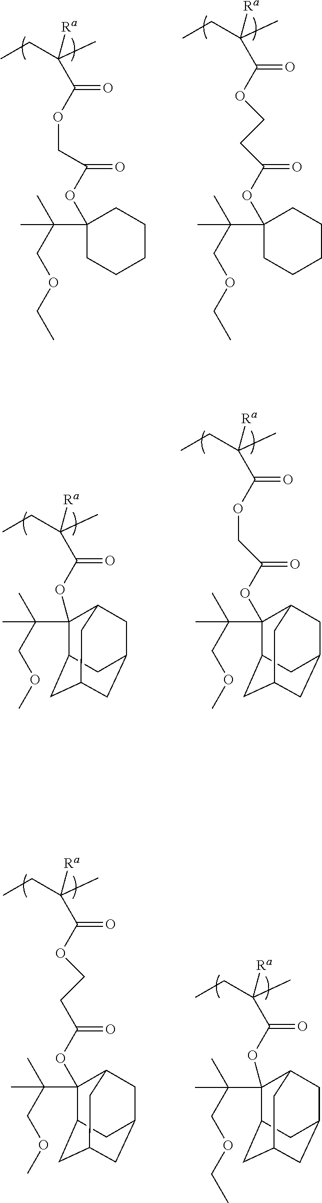

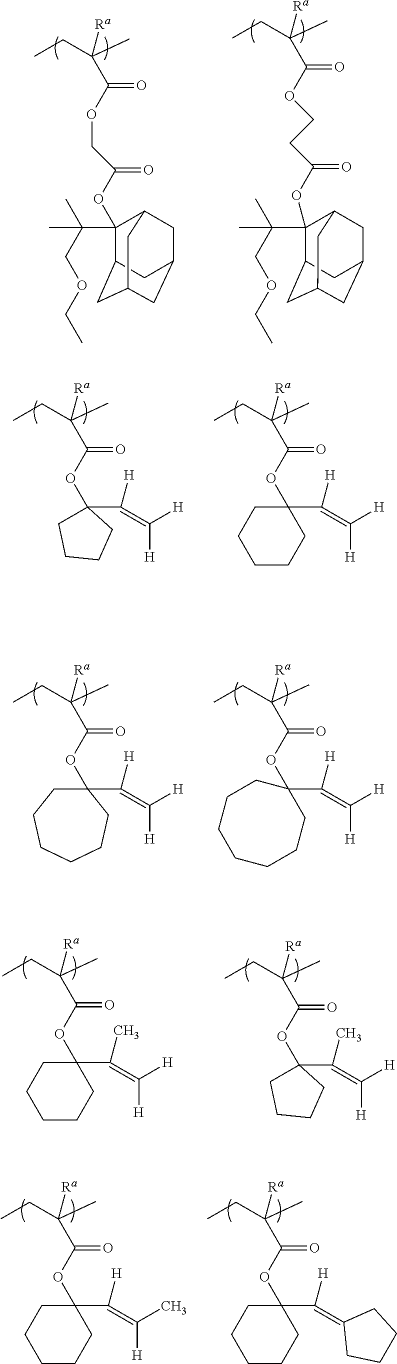

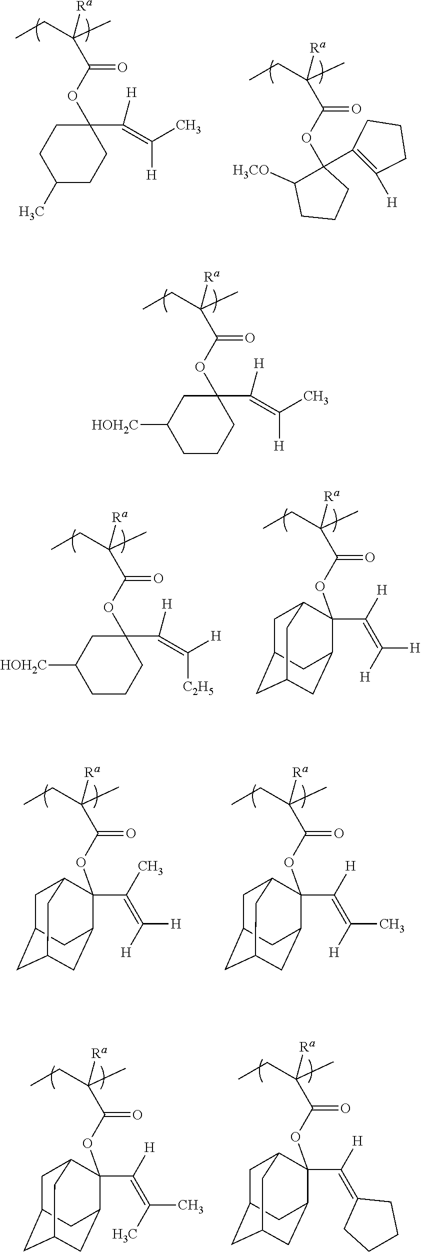

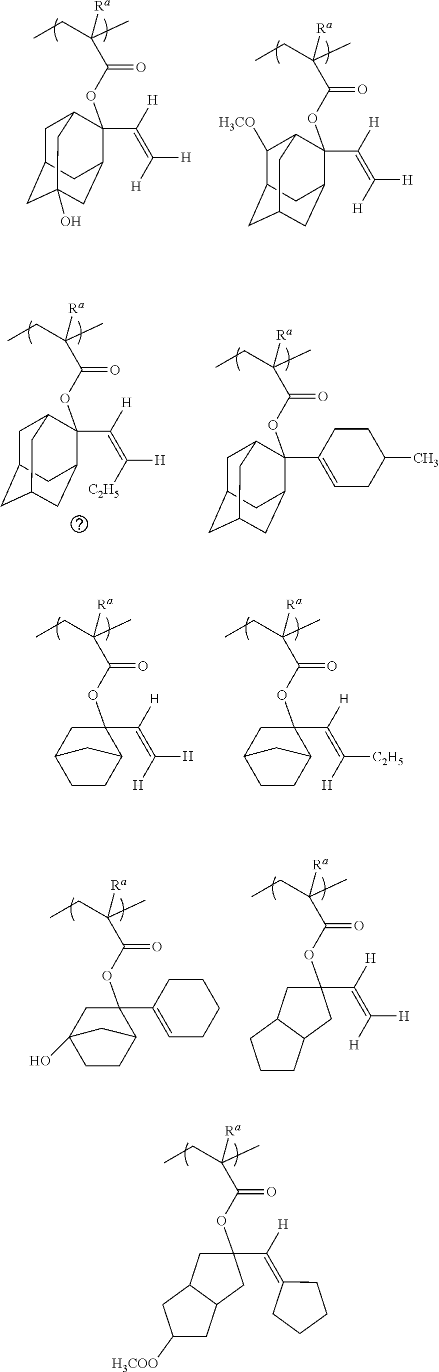

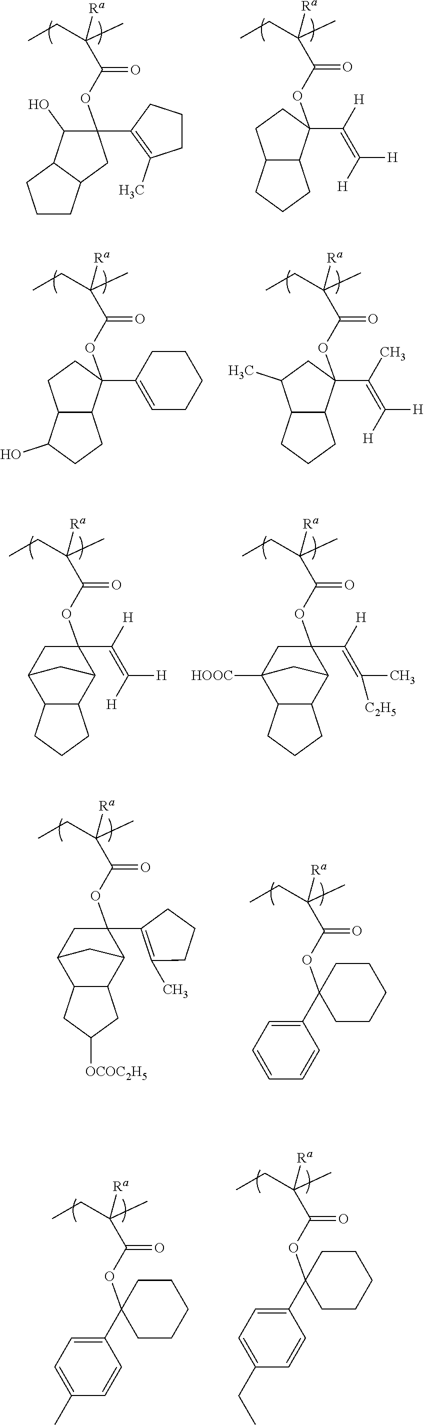

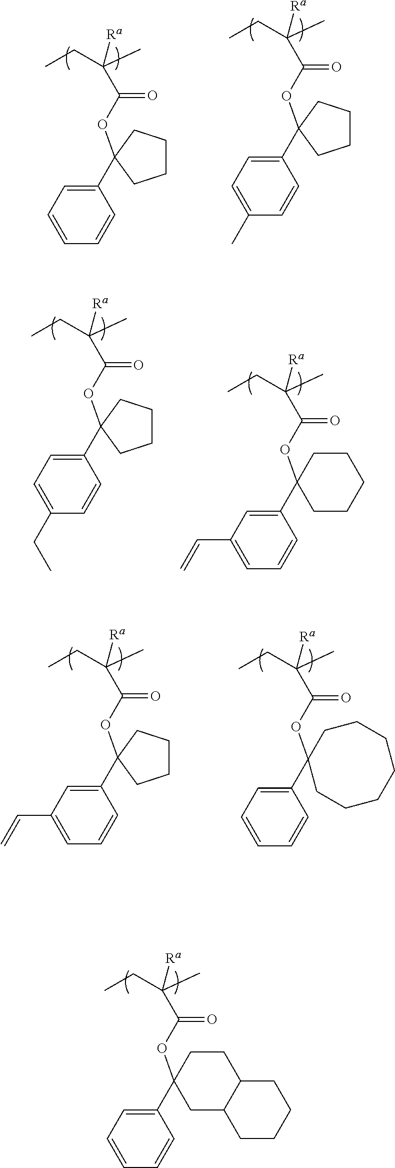

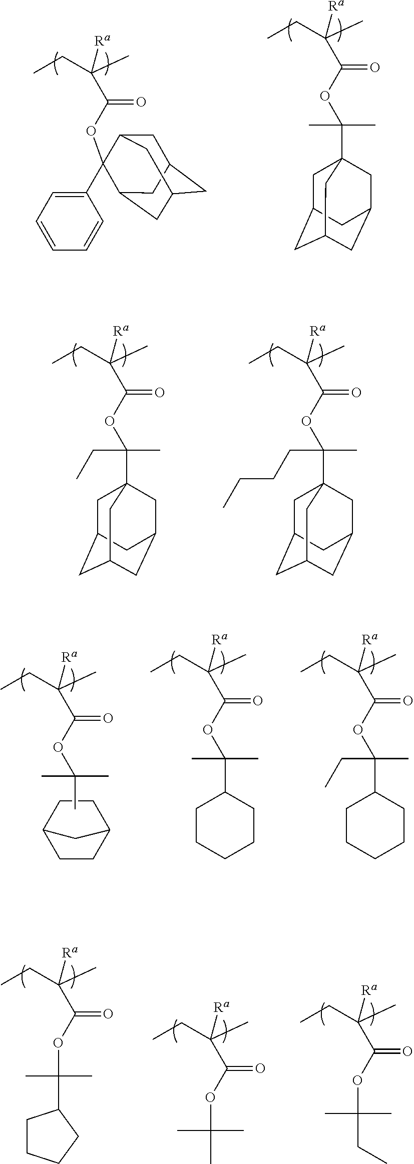

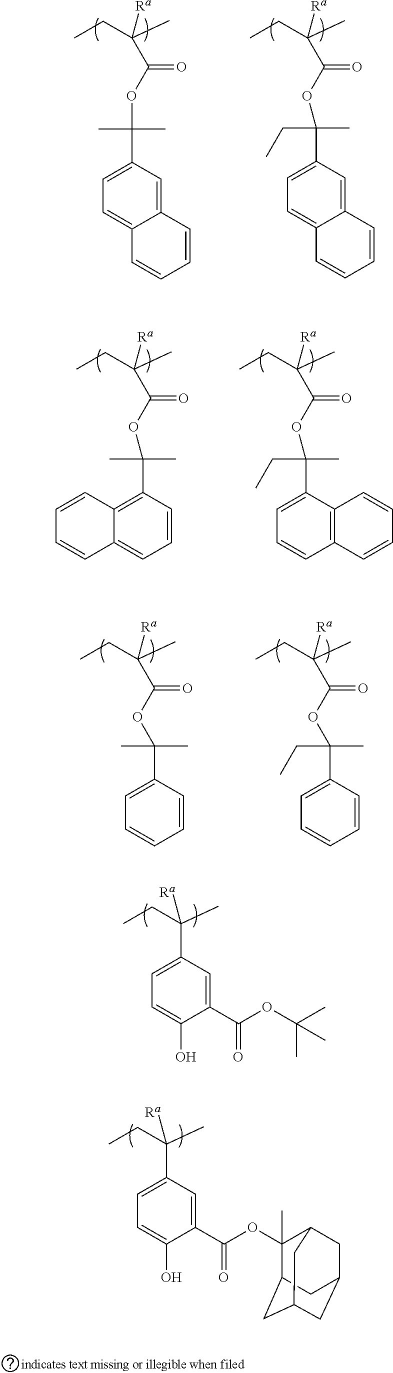

The constitutional unit (a1) included in the component (A1) may be used alone or two or more kinds thereof may be used.

Since the lithography characteristics (the sensitivity, the shape, and the like) are easily improved using electron beams or EUV, a constitutional unit represented by Formula (a1-1) is preferable as the constitutional unit (a1).

Among these, a constitutional unit that includes a constitutional unit represented by General Formula (a1-1-1) or a constitutional unit represented by General Formula (a1-1-2) is particularly preferable as the constitutional unit (a1).

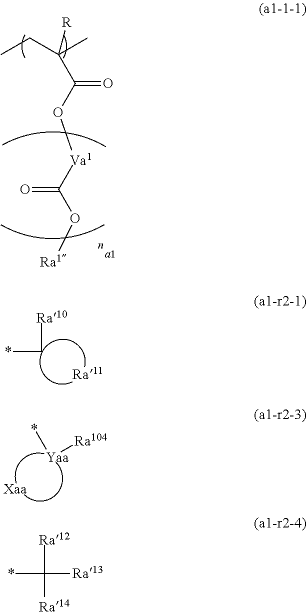

[In the formulae, Ra¹″ represents an acid-dissociable group represented by General Formula (a1-r2-1), (a1-r2-3), or (a1-r2-4). * represents a bonding site.]

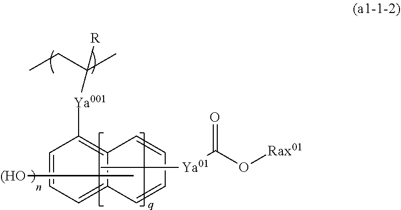

[In the formulae, R represents a hydrogen atom, an alkyl group having 1 to 5 carbon atoms, or a halogenated alkyl group having 1 to 5 carbon atoms. Ya⁰⁰¹ represents a single bond or a divalent linking group. Ya⁰¹ represents a single bond or a divalent linking group. Rax⁰¹ represents an acid-dissociable group represented by General Formula (a1-r2-1), (a1-r2-3), or (a1-r2-4). q represents an integer of 0 to 3. n represents an integer of 1 or more. However, n≤q×2+4 is satisfied.]

R, Va¹, and nₐ₁ in Formula (a1-1-1) each has the same definition as that for R, Va¹, and nₐ₁ in Formula (a1-1).

In Formula (a1-1-2), it is preferable that both Ya⁰⁰¹ and Ya⁰¹ represent a single bond.

The description of the acid-dissociable group represented by General Formula (a1-r2-1), (a1-r2-3), or (a1-r2-4) is the same as described above. In the description, from the viewpoint of suitably increasing the reactivity for EB or EUV, an acid-dissociable group represented by General Formula (a1-r2-1) or (a1-r2-4) is preferable.

An acid-dissociable group represented by General Formula (a1-r2-1) is more preferable as the acid-dissociable group in Formula (a1-1-1).

The proportion of the constitutional unit (a1) in the component (A1) is preferably in a range of 5% to 95% by mole, more preferably in a range of 10% to 90% by mole, still more preferably in a range of 30% to 70% by mole, and particularly preferably in a range of 40% to 60% by mole, with respect to the total amount (100% by mole) of all constitutional units constituting the component (A1).

In a case where the proportion of the constitutional unit (a1) is equal to or larger than the lower limit value of the above-described preferable range, lithography characteristics such as sensitivity, resolution, and roughness amelioration are improved. Further, in a case where the proportion of the constitutional unit (a1) is less than or equal to the upper limits of the above-described preferable ranges, the constitutional unit (a1) and other constitutional units can be balanced, and the lithography characteristics are improved.

Constitutional Unit (a10):

The constitutional unit (a10) is a constitutional unit represented by General Formula (a10-1).

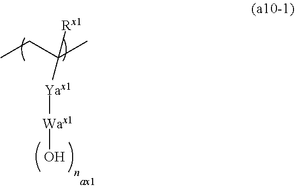

[In the formula, R^{x1} represents a hydrogen atom, an alkyl group having 1 to 5 carbon atoms, or a halogenated alkyl group having 1 to 5 carbon atoms. Ya^{x1} represents a single bond or a divalent linking group. Wa^{x1} represents an aromatic hydrocarbon group which may have a substituent. nₐₓ₁ represents an integer of 1 or greater.]

In Formula (a10-1), R^{x1} represents a hydrogen atom, an alkyl group having 1 to 5 carbon atoms, or a halogenated alkyl group having 1 to 5 carbon atoms.

R^{x1} represents preferably a hydrogen atom, an alkyl group having 1 to 5 carbon atoms, or a fluorinated alkyl group having 1 to 5 carbon atoms, and from the viewpoint of industrial availability, R represents more preferably a hydrogen atom, a methyl group, or trifluoromethyl group, still more preferably a hydrogen atom or a methyl group, and particularly preferably a hydrogen atom.

In Formula (a10-1), Ya^{x1} represents a single bond or a divalent linking group.

In the chemical formula, the divalent linking group as Ya^{x1} is not particularly limited, and suitable examples thereof include a divalent hydrocarbon group which may have a substituent and a divalent linking group having a heteroatom.

Ya^{x1} represents preferably a single bond, an ester bond [—C(═O)—O— or —O—C(═O)—], an ether bond (—O—), a linear or branched alkylene group, or a combination thereof and more preferably a single bond or an ester bond [—C(═O)—O— or —O—C(═O)—].

In Formula (a10-1), Wa^{x1} represents an aromatic hydrocarbon group which may have a substituent.

Examples of the aromatic hydrocarbon group as Wa^{x1} include a group in which (nₐₓ₁+1) hydrogen atoms have been removed from an aromatic ring which may have a substituent. The aromatic ring is not particularly limited as long as it is a cyclic conjugated system having (4n+2) π electrons. The aromatic ring has preferably 5 to 30 carbon atoms, more preferably 5 to 20 carbon atoms, still more preferably 6 to 15 carbon atoms, and particularly preferably 6 to 12 carbon atoms. Specific examples of the aromatic ring include aromatic hydrocarbon rings such as benzene, naphthalene, anthracene, and phenanthrene; and an aromatic heterocyclic ring obtained by substituting some carbon atoms constituting the above-described aromatic hydrocarbon ring with a heteroatom. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Further, examples of the aromatic hydrocarbon group as Wa^{x1} also include a group in which (nₐₓ₁+1) hydrogen atoms have been removed from an aromatic compound having an aromatic ring (for example, biphenyl or fluorene) which may have two or more substituents.

Among the examples, Wa^{x1} represents preferably a group in which (nₐₓ₁+1) hydrogen atoms have been removed from benzene, naphthalene, anthracene, or biphenyl, more preferably a group in which (nₐₓ₁+1) hydrogen atoms have been removed from benzene or naphthalene, and still more preferably a group in which (nₐₓ₁+1) hydrogen atoms have been removed from benzene.

The aromatic hydrocarbon group as Wa^{x1} may or may not have a substituent. Examples of substituents include an alkyl group, an alkoxy group, a halogen atom, and a halogenated alkyl group. Examples of the alkyl group, the alkoxy group, the halogen atom, and the halogenated alkyl group as the substituent include those described as the substituent of the cyclic aliphatic hydrocarbon group as Ya^{x1}. The substituent is preferably a linear or branched alkyl group having 1 to 5 carbon atoms, more preferably a linear or branched alkyl group having 1 to 3 carbon atoms, still more preferably an ethyl group or a methyl group, and particularly preferably a methyl group. It is preferable that the aromatic hydrocarbon group as Wa^{x1} have no substituent.

In Formula (a10-1), nₐₓ₁ represents an integer of 1 or greater, preferably an integer of 1 to 10, more preferably an integer of 1 to 5, still more preferably 1, 2, or 3, and particularly preferably 1 or 2.

Specific examples of the constitutional unit (a10) represented by Formula (a10-1) are described below.

In the formulae shown below, R^{α} represents a hydrogen atom, a methyl group, or a trifluoromethyl group.

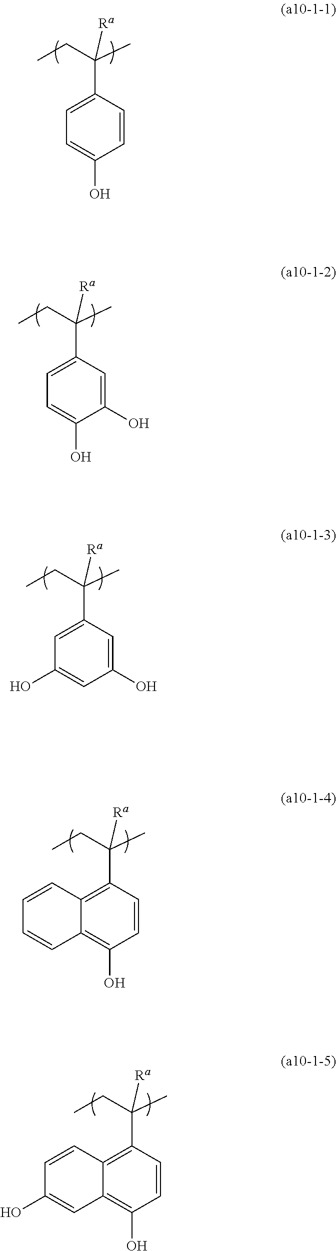

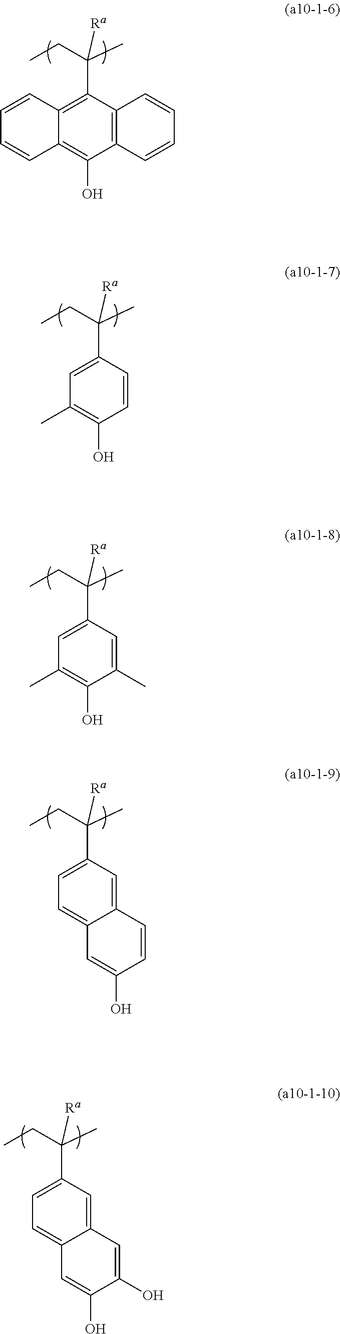

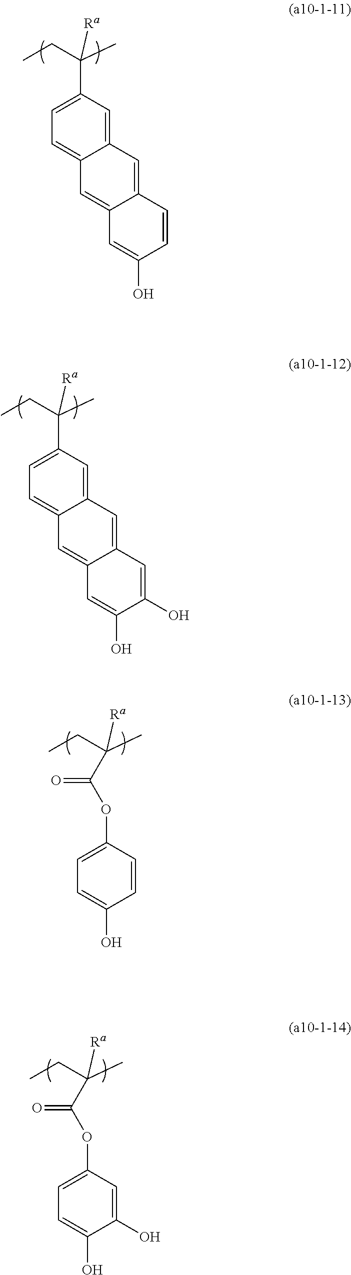

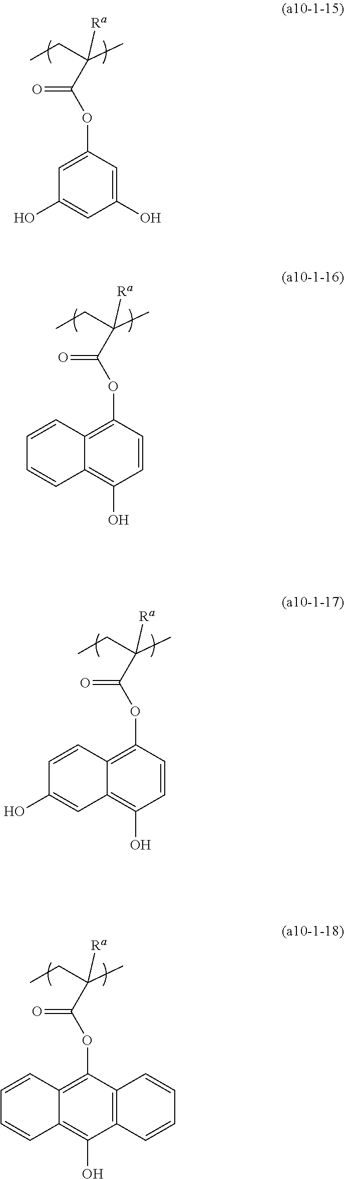

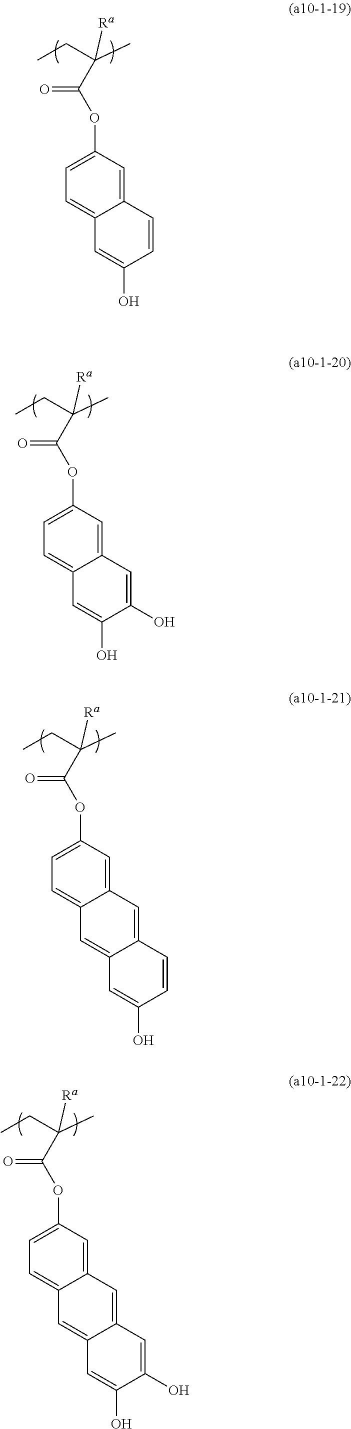

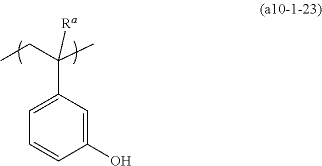

The constitutional unit (a10) included in the component (A1) may be used alone or two or more kinds thereof may be used.

In a case where the component (A1) has the constitutional unit (a10), the proportion of the constitutional unit (a10) in the component (A1) is preferably in a range of 20% to 80% by mole, more preferably in a range of 25% to 70% by mole, and still more preferably in a range of 25% to 60% by mole with respect to the total amount (100% by mole) of all constitutional units constituting the component (A1).

In a case where the proportion of the constitutional unit (a10) is set to be greater than or equal to the above-described lower limits, the sensitivity is likely to be enhanced. Meanwhile, in a case where the proportion thereof is set to be less than or equal to the above-described upper limits, the constitutional unit (a10) and other constitutional units are likely to be balanced.

### Constitutional Unit (a2):

The component (A1) may have a constitutional unit (a2) containing a lactone-containing cyclic group (here, constitutional units corresponding to the constitutional unit (a1) are excluded).

In a case where the component (A1) is used to form a resist film, the lactone-containing cyclic group of the constitutional unit (a2) is effective for increasing the adhesiveness of the resist film to the substrate. Further, in a case where the component (A1) contains the constitutional unit (a2), the lithography characteristics and the like are improved due to the effects of appropriately adjusting the acid diffusion length, increasing the adhesiveness of the resist film to the substrate, and appropriately adjusting the solubility during the development.

The term “lactone-containing cyclic group” indicates a cyclic group that has a ring (lactone ring) containing —O—C(═O)— in the ring skeleton. In a case where the lactone ring is counted as the first ring and the group contains only the lactone ring, the group is referred to as a monocyclic group. Further, in a case where the group has other ring structures, the group is referred to as a polycyclic group regardless of the structures. The lactone-containing cyclic group may be a monocyclic group or a polycyclic group.

The lactone-containing cyclic group in the constitutional unit (a2) is not particularly limited, and an optional constitutional unit can be used. Specific examples thereof include groups each represented by General Formulae (a2-r-1) to (a2-r-7).

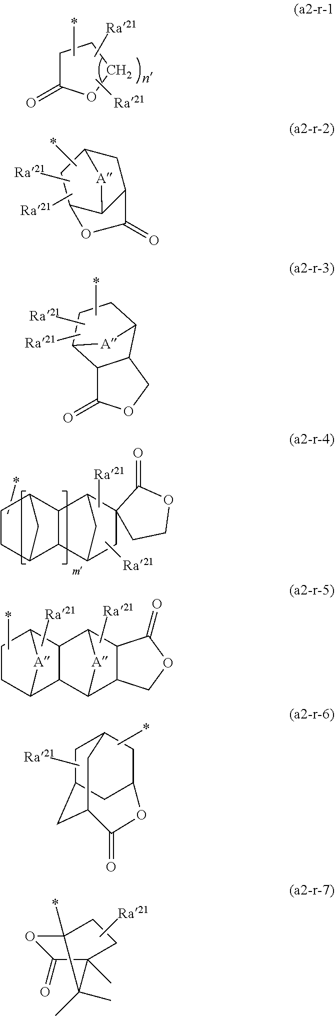

[In the formulae, Ra′²¹'s each independently represents a hydrogen atom, an alkyl group, an alkoxy group, a halogen atom, a halogenated alkyl group, a hydroxyl group, —COOR″, —OC(═O)R″, a hydroxyalkyl group, or a cyano group, R″ represents a hydrogen atom, an alkyl group, or a lactone-containing cyclic group, A″ represents an alkylene group having 1 to 5 carbon atoms which may have an oxygen atom (—O—) or a sulfur atom (—S—), an oxygen atom, or a sulfur atom, n′ represents an integer of 0 to 2, and m′ is 0 or 1. * represents a bonding site (the same applies hereinafter).]

In General Formulae (a2-r-1) to (a2-r-7), it is preferable that the alkyl group as Ra′²¹ be an alkyl group having 1 to 6 carbon atoms. It is preferable that the alkyl group be linear or branched. Specific examples thereof include a methyl group, an ethyl group, a propyl group, an isopropyl group, an n-butyl group, an isobutyl group, a tert-butyl group, a pentyl group, an isopentyl group, a neopentyl group, and a hexyl group. Among these, a methyl group or ethyl group is preferable, and a methyl group is particularly preferable.

It is preferable that the alkoxy group as Ra′²¹ be an alkoxy group having 1 to 6 carbon atoms. Further, it is preferable that the alkoxy group be linear or branched. Specific examples of the alkoxy groups include a group formed by linking the above-described alkyl group exemplified as the alkyl group represented by Ra′²¹ to an oxygen atom (—O—).

As the halogen atom as Ra′²¹, a fluorine atom is preferable.

Examples of the halogenated alkyl group as Ra′²¹ include groups in which some or all hydrogen atoms in the alkyl group as Ra′²¹ are substituted with the halogen atoms. As the halogenated alkyl group, a fluorinated alkyl group is preferable, and a perfluoroalkyl group is particularly preferable.

In —COOR″ and —OC(═O))R″ as Ra′²¹, any R″ represents a hydrogen atom, an alkyl group, or a lactone-containing cyclic group.

The alkyl group as R″ may be linear, branched, or cyclic, and preferably has 1 to 15 carbon atoms.

In a case where R″ represents a linear or branched alkyl group, an alkyl group having 1 to 10 carbon atoms is preferable, an alkyl group having 1 to 5 carbon atoms is more preferable, and a methyl group or an ethyl group is particularly preferable.

In a case where R″ represents a cyclic alkyl group, the number of carbon atoms thereof is preferably in a range of 3 to 15, more preferably in a range of 4 to 12, and most preferably in a range of 5 to 10. Specific examples thereof include groups in which one or more hydrogen atoms have been removed from a monocycloalkane, which may or may not be substituted with a fluorine atom or a fluorinated alkyl group; and groups in which one or more hydrogen atoms have been removed from a polycycloalkane such as bicycloalkane, tricycloalkane, or tetracycloalkane. More specific examples thereof include groups in which one or more hydrogen atoms have been removed from a monocycloalkane such as cyclopentane or cyclohexane; and groups in which one or more hydrogen atoms have been removed from a polycycloalkane such as adamantane, norbornane, isobornane, tricyclodecane, or tetracyclododecane.

Examples of the lactone-containing cyclic group as R″ include the same groups as those for the groups each represented by General Formulae (a2-r-1) to (a2-r-7).

As the hydroxyalkyl group as Ra′²¹, a hydroxyalkyl group having 1 to 6 carbon atoms is preferable, and specific examples thereof include a group in which at least one hydrogen atom in the alkyl group as Ra′²¹ has been substituted with a hydroxyl group.

Among the examples, it is preferable that Ra′²¹'s each independently represent a hydrogen atom or a cyano group.

In General Formulae (a2-r-2), (a2-r-3) and (a2-r-5), as the alkylene group having 1 to 5 carbon atoms as A″, a linear or branched alkylene group is preferable, and examples thereof include a methylene group, an ethylene group, an n-propylene group, and an isopropylene group. In a case where the alkylene group has an oxygen atom or a sulfur atom, specific examples thereof include groups in which —O— or —S— is interposed in the terminal of the alkylene group or between the carbon atoms of the alkylene group. Further, examples thereof include —O—CH₂—, —CH₂—O—CH₂—, —S—CH₂—, and —CH₂—S—CH₂—. A″ represents preferably an alkylene group having 1 to 5 carbon atoms or —O—, more preferably an alkylene group having 1 to 5 carbon atoms, and most preferably a methylene group.

Specific examples of the groups each represented by General Formulae (a2-r-1) to (a2-r-7) are shown below.

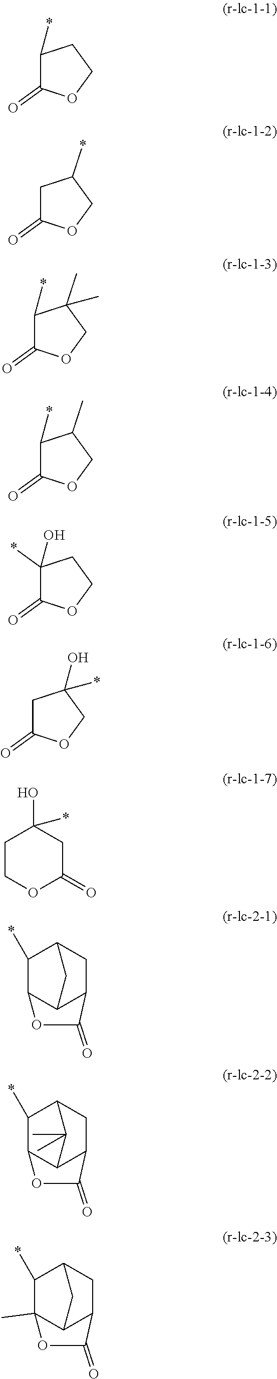

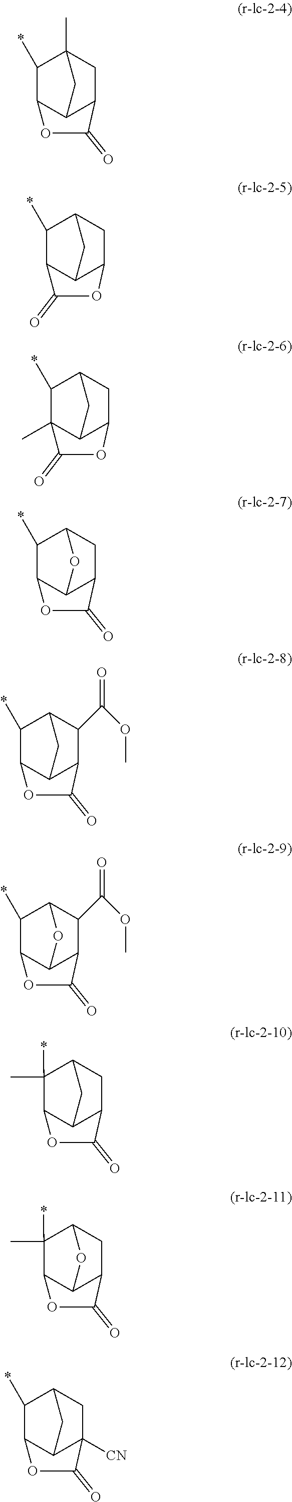

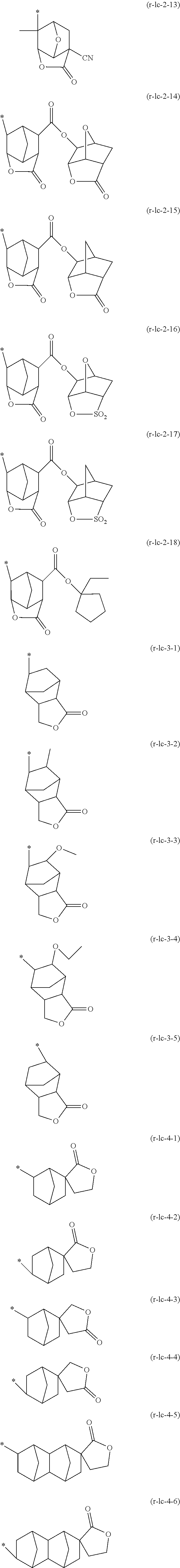

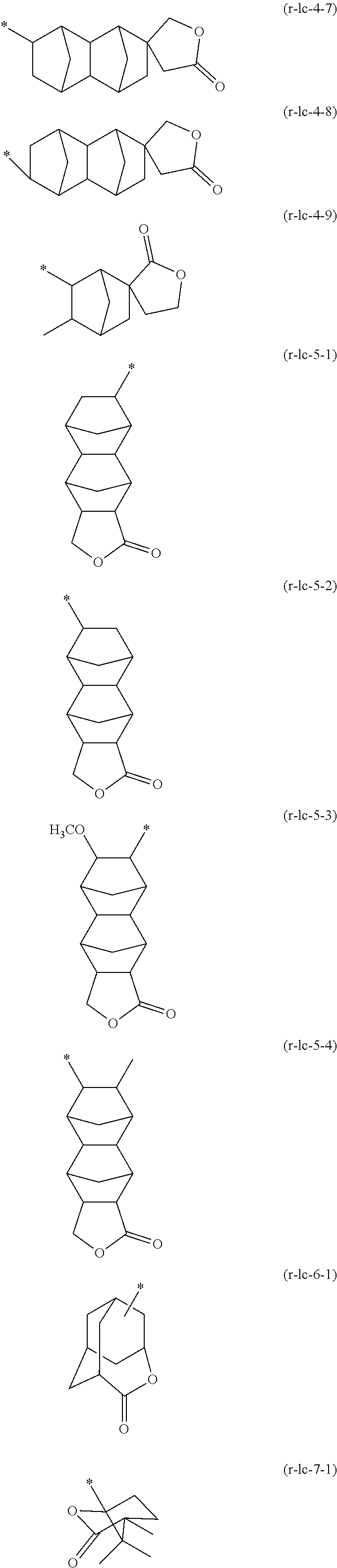

As the constitutional unit (a2), a constitutional unit derived from acrylic acid ester in which the hydrogen atom bonded to the carbon atom at the α-position may be substituted with a substituent is preferable.

It is preferable that such a constitutional unit (a2) be a constitutional unit represented by General Formula (a2-1).

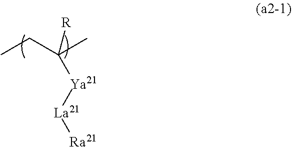

[In the formula, R represents a hydrogen atom, an alkyl group having 1 to 5 carbon atoms, or a halogenated alkyl group having 1 to 5 carbon atoms. Ya²¹ represents a single bond or a divalent linking group. La²¹ represents —O—, —COO—, —CON(R′)—, —OCO—, —CONHCO—, or —CONHCS—, and R′ represents a hydrogen atom or a methyl group. In a case where La²¹ represents —O—, Ya²¹ does not represent —CO—. Ra²¹ represents a lactone-containing cyclic group.]

In Formula (a2-1), R has the same definition as described above. R represents preferably a hydrogen atom, an alkyl group having 1 to 5 carbon atoms, or a fluorinated alkyl group having 1 to 5 carbon atoms and particularly preferably a hydrogen atom or a methyl group from the viewpoint of the industrial availability.

In Formula (a2-1), the divalent linking group as Ya²¹ is not particularly limited, and suitable examples thereof include a divalent hydrocarbon group which may have a substituent and a divalent linking group having a heteroatom.

It is preferable that Ya²¹ represent a single bond, an ester bond [—C(═O)—O—], an ether bond (—O—), a linear or branched alkylene group, or a combination thereof.

In Formula (a2-1), it is preferable that Ya²¹ represent a single bond and La²¹ represents —COO— or —OCO—.

In Formula (a2-1), Ra²¹ represents a lactone-containing cyclic group.

Suitable examples of the lactone-containing cyclic group as Ra²¹ include groups each represented by General Formulae (a2-r-1) to (a2-r-7).

The constitutional unit (a2) included in the component (A1) may be used alone or two or more kinds thereof may be used.

In a case where the component (A1) has the constitutional unit (a2), the proportion of the constitutional unit (a2) is preferably in a range of 1% to 20% by mole, more preferably in a range of 1% to 15% by mole, and still more preferably in a range of 1% to 10% by mole with respect to the total amount (100% by mole) of all constitutional units constituting the component (A1).

In a case where the proportion of the constitutional unit (a2) is set to be greater than or equal to the lower limits of the above-described preferable ranges, the effect to be obtained by allowing the component (A1) to have the constitutional unit (a2) is sufficiently obtained by the above-described effects. Further, in a case where the proportion thereof is set to be less than or equal to the upper limits of the above-described preferable ranges, the constitutional unit (a2) and other constitutional units can be balanced, and the lithography characteristics are improved.

### Constitutional Unit (a8):

The constitutional unit (a8) is a constitutional unit derived from a compound represented by General Formula (a8-1).

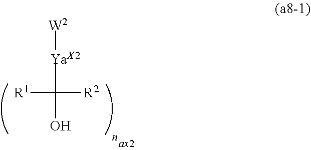

[In the formula, W² represents a polymerizable group-containing group. Ya^{x2} represents a single bond or an (nₐₓ₂+1)-valent linking group. Ya^{x2} and W² may form a condensed ring. R¹ represents a fluorinated alkyl group having 1 to 12 carbon atoms. R² represents an organic group having 1 to 12 carbon atoms, which may have a fluorine atom, or a hydrogen atom. R² and Ya^{x2} may be bonded to each other to form a ring structure. nₐₓ₂ represents an integer of 1 to 3.]

The term “polymerizable group” in the polymerizable group-containing group as W² denotes a group that enables a compound containing a polymerizable group to be polymerized by radical polymerization or the like, which is, for example, a group having a multiple bond between carbon atoms, such as an ethylenic double bond.

The polymerizable group-containing group may be a group formed of only a polymerizable group or a group formed of a polymerizable group and a group other than the polymerizable group. Examples of the group other than the polymerizable group include a divalent hydrocarbon group which may have a substituent and a divalent linking group having a heteroatom.

Suitable examples of the polymerizable group-containing group include a group represented by Chemical Formula: C(R^{X11})(R^{X12})=C(R^{X13})-Ya^{x0}.

In the chemical formula, R^{X11}, R^{X12}, and R^{X13} each represents a hydrogen atom, an alkyl group having 1 to 5 carbon atoms, or a halogenated alkyl group having 1 to 5 carbon atoms, and Ya^{x0} represents a single bond or a divalent linking group.

Examples of the condensed ring formed by Ya^{x2} and W² include a condensed ring formed by a polymerizable group of the W² moiety with Ya^{x2} and a condensed ring formed by a group other than the polymerizable group of the W² moiety with Ya^{x2}.

The condensed ring formed by Ya^{x2} and W² may have a substituent.

Specific examples of the constitutional unit (a8) are shown below.

In the following formulae, R^{α} represents a hydrogen atom, a methyl group, or a trifluoromethyl group.

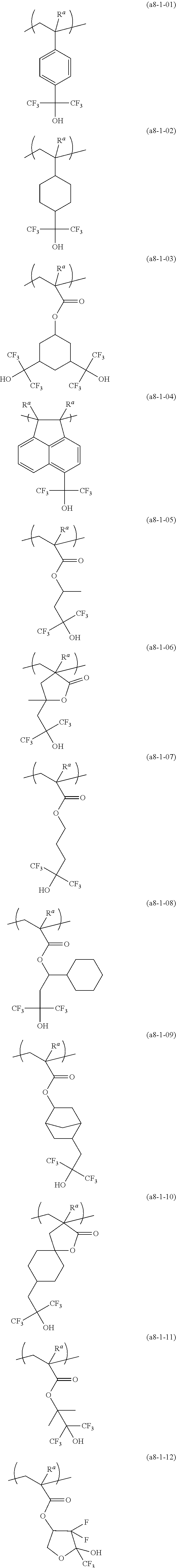

Among the examples, the constitutional unit (a8) is preferably at least one selected from the group consisting of constitutional units each represented by Chemical Formulae (a8-1-01) to (a8-1-04), (a8-1-06), (a8-1-08), (a8-1-09), and (a8-1-10) and more preferably at least one selected from the group consisting of constitutional units each represented by Chemical Formulae (a8-1-01) to (a8-1-04) and (a8-1-09).

The constitutional unit (a8) contained in the component (A1) may be used alone or two or more kinds thereof may be used.

The proportion of the constitutional unit (a8) in the component (A1) is preferably in a range of 0% to 50% by mole and more preferably in a range of 0% to 30% by mole with respect to the total amount (100% by mole) of all constitutional units constituting the component (A1).

The component (A1) contained in the resist composition may be used alone or in combination of two or more kinds thereof.

Examples of the component (A1) include a polymer compound having a repeating structure of the constitutional unit (a0) and the constitutional unit (a1); and a polymer compound having a repeating structure of the constitutional unit (a0), the constitutional unit (a1), and the constitutional unit (a10).

Such a component (A1) can be produced by dissolving a monomer, from which each constitutional unit is derived, in a polymerization solvent and adding a radical polymerization initiator such as azobisisobutylonitrile (AIBN) or dimethyl azobisisobutyrate (for example, V-601) to the solution so that the polymerization is carried out.

Alternatively, such a component (A1) can be produced by dissolving, in a polymerization solvent, a monomer from which the constitutional unit (a0) is derived and a monomer from which the constitutional unit (a1) and the constitutional unit (a10) are derived, and adding the above-described radical polymerization initiator thereto to perform polymerization, and performing a deprotection reaction.

Further, a —C(CF₃)₂—OH group may be introduced to the terminal during the polymerization using a combination of chain transfer agents such as HS—CH₂—CH₂—CH₂—C(CF₃)₂—OH. As described above, a copolymer into which a hydroxyalkyl group, formed by substitution of some hydrogen atoms in the alkyl group with fluorine atoms, has been introduced is effective for reducing development defects and reducing line edge roughness (LER: uneven irregularities of a line side wall).

The weight-average molecular weight (Mw)(in terms of polystyrene according to gel permeation chromatography (GPC)) of the component (A1) is not particularly limited, but is preferably in a range of 1000 to 50000, more preferably in a range of 5000 to 40000, and still more preferably in a range of 10000 to 30000.

In a case where the Mw of the component (A1) is less than or equal to the upper limits of the above-described preferable ranges, the resist composition exhibits sufficiently satisfactory solubility in a resist solvent to be used as a resist. On the contrary, in a case where the Mw of the component (A1) is greater than or equal to the lower limits of the above-described preferable ranges, the dry etching resistance and the cross-sectional shape of the resist pattern are excellent.

Further, the dispersity (Mw/Mn) of the component (A1) is not particularly limited, but is preferably in a range of 1.0 to 4.0, more preferably in a range of 1.0 to 3.0, and particularly preferably in a range of 1.0 to 2.0. Further, Mn represents the number-average molecular weight.

### In Regard to Component (A2)

In the resist composition of the present embodiment, a base material component (hereinafter, also referred to as “component (A2)”) which does not correspond to the component (A1) and whose solubility in a developing solution is changed by the action of an acid may be used in combination as the component (A).

The component (A2) is not particularly limited and may be optionally selected from a plurality of components of the related art known as base material components for a chemically amplified resist composition and used.

As the component (A2), a polymer compound or a low-molecular-weight compound may be used alone or in combination of two or more kinds thereof.

The proportion of the component (A1) in the component (A) is preferably 25% by mass or greater, more preferably 50% by mass or greater, and still more preferably 75% by mass or greater, and may be 100% by mass with respect to the total mass of the component (A). In a case where the proportion thereof is 25% by mass or greater, a resist pattern having various excellent lithography characteristics such as high sensitivity, high resolution, and improved roughness is likely to be formed.

In the resist composition of the present embodiment, the content of the component (A) may be adjusted according to the thickness of the resist film intended to be formed.

### <Other Components>

The resist composition according to the present embodiment may further contain other components in addition to the component (A) described above. Examples of the other components include a component (B), a component (D), a component (E), a component (F), and a component(S), which are described below.

### <<Acid Generator Component (B)>>

The resist composition according to the present embodiment may further contain an acid generator component (B) which generates an acid upon light exposure.

The component (B) is not particularly limited, and those which have been suggested so far as an acid generator for a chemically amplified resist composition in the related art can be used.

Examples of the acid generator include various acid generators, for example, onium salt-based acid generators such as iodonium salts and sulfonium salts; oxime sulfonate-based acid generators; diazomethane-based acid generators such as bisalkyl or bisaryl sulfonyl diazomethanes and poly(bis-sulfonyl)diazomethanes; nitrobenzyl sulfonate-based acid generators, iminosulfonate-based acid generators, and disulfone-based acid generators.

Examples of the onium salt-based acid generators include a compound represented by General Formula (b-1) (hereinafter, also referred to as “component (b-1)”), a compound represented by General Formula (b-2) (hereinafter, also referred to as “component (b-2)”), and a compound represented by General Formula (b-3) (hereinafter, also referred to as “component (b-3)”).

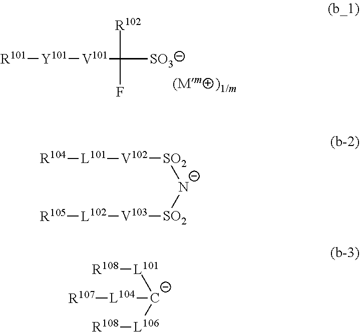

[In the formulae, R¹⁰¹ and R¹⁰⁴ to R¹⁰⁸ each independently represents a cyclic group which may have a substituent, a chain-like alkyl group which may have a substituent, or a chain-like alkenyl group which may have a substituent. R¹⁰⁴ and R¹⁰⁵ may be bonded to each other to form a ring structure. R¹⁰² represents a fluorinated alkyl group having 1 to 5 carbon atoms or a fluorine atom. Y¹⁰¹ represents a divalent linking group having an oxygen atom or a single bond. V¹⁰¹ to V¹⁰³ each independently represents a single bond, an alkylene group, or a fluorinated alkylene group. L¹⁰¹ and L¹⁰² each independently represents a single bond or an oxygen atom. L¹⁰³ to L¹⁰⁵ each independently represents a single bond, —CO—, or —SO₂—. m represents an integer of 1 or greater, and M′^{m+} represents an m-valent onium cation.]

### {Anion Moiety}

Anions in Component (b-1)

In Formula (b-1), R¹⁰¹ represents a cyclic group which may have a substituent, a chain-like alkyl group which may have a substituent, or a chain-like alkenyl group which may have a substituent.

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group with no aromaticity. Further, the aliphatic hydrocarbon group may be saturated or unsaturated. In general, it is preferable that the aliphatic hydrocarbon group be saturated.

The aromatic hydrocarbon group as R¹⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring of the aromatic hydrocarbon group as R¹⁰¹ include benzene, fluorene, naphthalene, anthracene, phenanthrene, biphenyl, and an aromatic heterocyclic ring in which some carbon atoms constituting any of these aromatic rings are substituted with heteroatoms. Examples of the heteroatom in the aromatic heterocyclic rings include an oxygen atom, a sulfur atom, and a nitrogen atom.

Specific examples of the aromatic hydrocarbon group as R¹⁰¹ include a group in which one hydrogen atom has been removed from the aromatic ring (an aryl group such as a phenyl group or a naphthyl group) and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, I-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R¹⁰¹ include an aliphatic hydrocarbon group having a ring in the structure thereof.

Examples of the aliphatic hydrocarbon group having a ring in the structure thereof include an alicyclic hydrocarbon group (a group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, tricyclodecane, or tetracyclododecane; and a polycycloalkane having a condensed ring polycyclic skeleton such as a cyclic group having a steroid skeleton are preferable as the polycycloalkane.

Among these examples, as the cyclic aliphatic hydrocarbon group as R¹⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is still more preferable, and an adamantyl group is particularly preferable.

The linear aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and most preferably 1 to 3 carbon atoms. As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group which may be bonded to the alicyclic hydrocarbon group has preferably 2 to 10 carbon atoms, more preferably 3 to 6 carbon atoms, still more preferably 3 or 4 carbon atoms, and most preferably 3 carbon atoms. As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃), and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

Further, the cyclic hydrocarbon group as R¹⁰¹ may have a heteroatom such as a heterocyclic ring. Specific examples thereof include lactone-containing cyclic groups each represented by General Formulae (a2-r-1) to (a2-r-7), —SO₂-containing cyclic groups each represented by General Formulae (b5-r-1) to (b5-r-4), and other heterocyclic groups each represented by Chemical Formulae (r-hr-1) to (r-hr-16). In the formulae, * represents a bonding site with respect to Y¹⁰¹ in Formula (b-1).

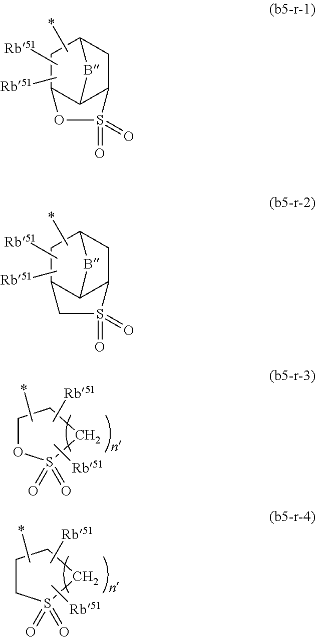

[In the formulae, Rb′⁵¹'s each independently represents a hydrogen atom, an alkyl group, an alkoxy group, a halogen atom, a halogenated alkyl group, a hydroxyl group, —COOR″, —OC(═O)R″, a hydroxyalkyl group, or a cyano group, R″ represents a hydrogen atom, an alkyl group, a lactone-containing cyclic group, or a —SO₂-containing cyclic group, B″ represents an alkylene group having 1 to 5 carbon atoms which may have an oxygen atom or a sulfur atom, an oxygen atom, or a sulfur atom, and n′ represents an integer of 0 to 2. * represents a bonding site.]

Rb′⁵¹, B″, and n′ in General Formulae (b5-r-1) to (b5-r-4) each has the same definition as that for Rb′⁵¹, B″, and n′ in General Formulae (a0-s-1) to (a0-s-4).

Specific examples of the groups each represented by General Formulae (b5-r-1) to (b5-r-4) are shown below. In the formulae shown below, “Ac” represents an acetyl group.

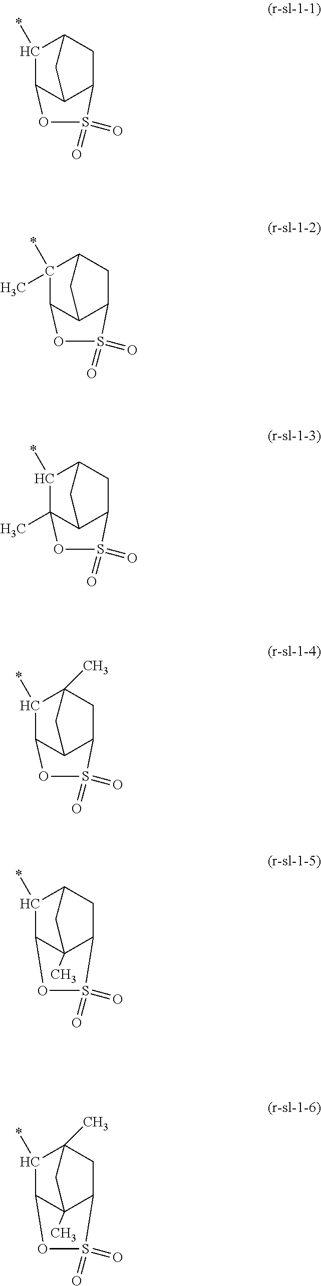

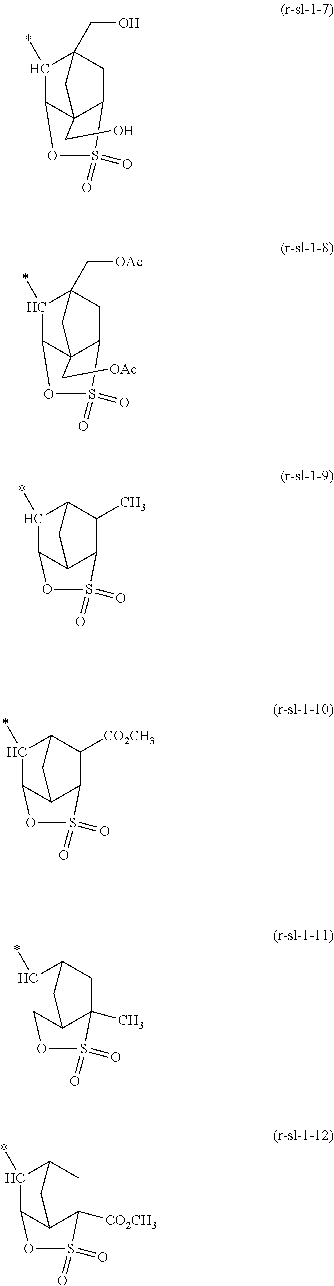

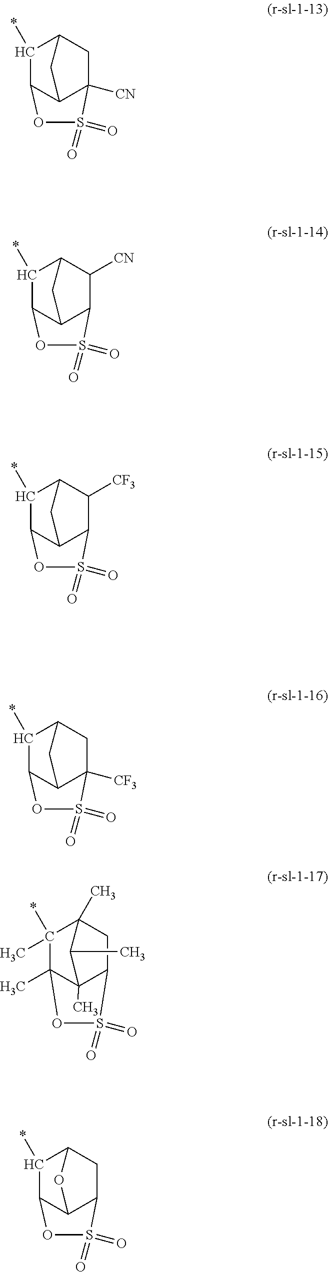

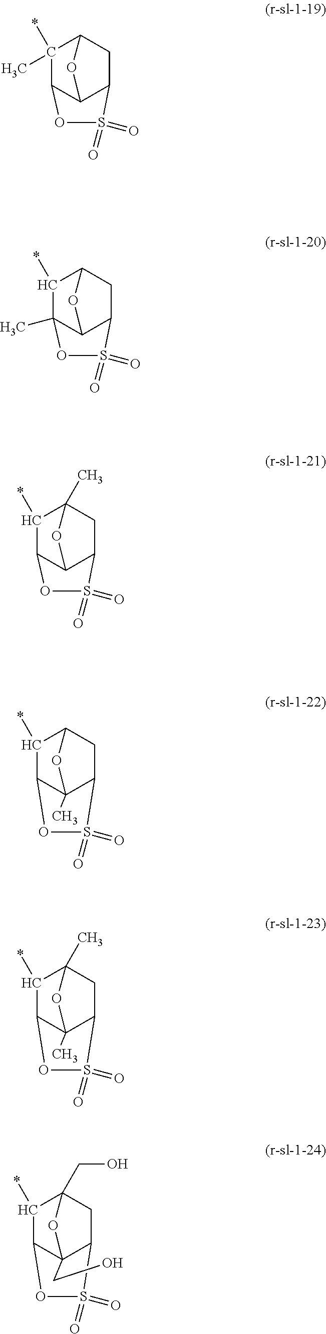

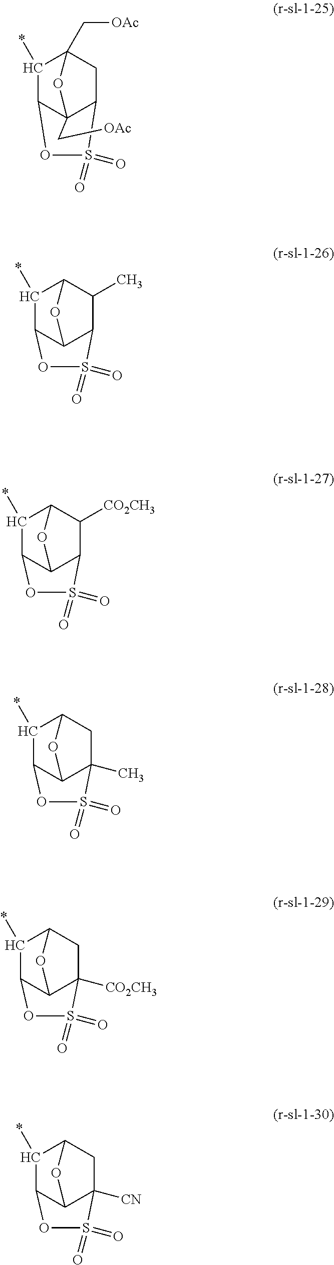

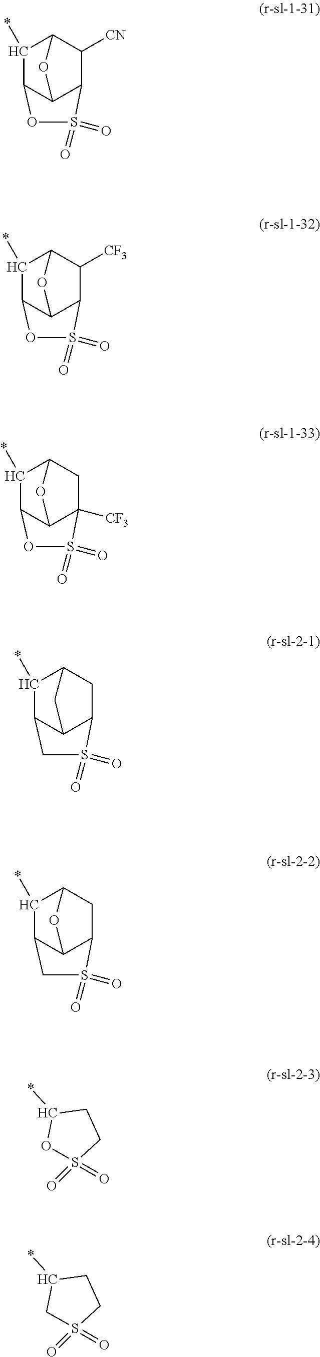

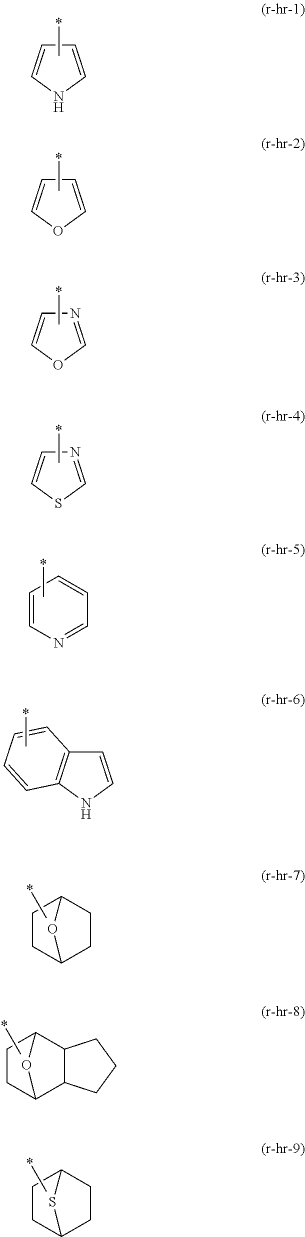

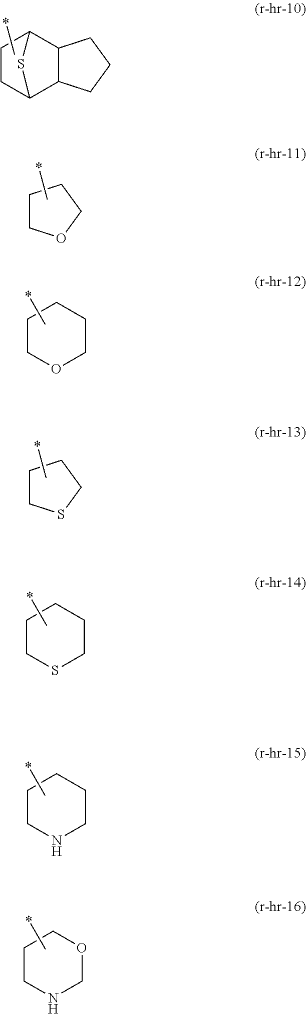

Examples of the substituent for the cyclic group as R¹⁰¹ include an alkyl group, an alkoxy group, a halogen atom, a halogenated alkyl group, a hydroxyl group, a carbonyl group, and a nitro group.

As the alkyl group as the substituent, an alkyl group having 1 to 5 carbon atoms is preferable, and a methyl group, an ethyl group, a propyl group, an n-butyl group, or a tert-butyl group is most preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

Examples of the halogen atom as the substituent include a fluorine atom, a chlorine atom, a bromine atom, and an iodine atom. Among these, a fluorine atom is preferable.

Examples of the above-described halogenated alkyl group as the substituent include a group in which some or all hydrogen atoms in an alkyl group having 1 to 5 carbon atoms such as a methyl group, an ethyl group, a propyl group, an n-butyl group, or a tert-butyl group are substituted with the above-described halogen atoms.

The carbonyl group as the substituent is a group that substitutes a methylene group (—CH₂—) constituting the cyclic hydrocarbon group.

The cyclic hydrocarbon group as RIO may be a condensed cyclic group having a condensed ring in which an aliphatic hydrocarbon ring and an aromatic ring are condensed. Examples of the condensed ring include those obtained by condensing one or more aromatic rings with a polycycloalkane having a crosslinked ring polycyclic skeleton. Specific examples of the crosslinked ring polycycloalkane include a bicycloalkane such as bicyclo[2.2.1]heptane (norbornane) and bicyclo[2.2.2]octane. As the condensed cyclic group, a group having a condensed ring in which two or three aromatic rings are condensed with a bicycloalkane is preferable, and a group having a condensed ring in which two or three aromatic rings are condensed with bicyclo[2.2.2]octane is more preferable. Specific examples of the condensed cyclic group as R¹⁰¹ include those represented by Formulae (r-br-1) and (r-br-2). In the formulae, * represents a bonding site with respect to Y¹⁰¹ in Formula (b-1).

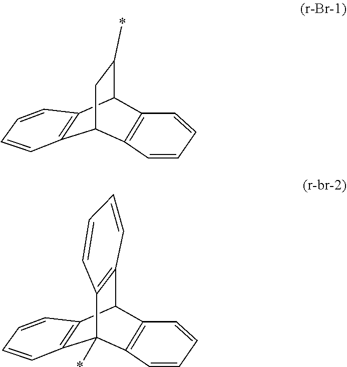 [SMILES: *C1CC2c3ccccc3C1c1ccccc12 ; *C12c3ccccc3C(c3ccccc31)c1ccccc12]

Examples of the substituent that the condensed cyclic group as R¹⁰¹ may have include an alkyl group, an alkoxy group, a halogen atom, a halogenated alkyl group, a hydroxyl group, a carbonyl group, a nitro group, an aromatic hydrocarbon group, and an alicyclic hydrocarbon group.

Examples of the alkyl group, the alkoxy group, the halogen atom, and the halogenated alkyl group as the substituent of the condensed cyclic group include those exemplified as the substituent of the cyclic group as R¹⁰¹.

Examples of the aromatic hydrocarbon group as the substituent of the condensed cyclic group include a group in which one hydrogen atom has been removed from the aromatic ring (an aryl group such as a phenyl group or a naphthyl group), a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group), and a heterocyclic group represented by any of Formulae (r-hr-1) to (r-hr-6).

Examples of the alicyclic hydrocarbon group as the substituent of the condensed cyclic group include a group in which one hydrogen atom has been removed from a monocycloalkane such as cyclopentane or cyclohexane, a group in which one hydrogen atom has been removed from a polycycloalkane such as adamantane, norbornane, isobornane, tricyclodecane, or tetracyclododecane, a lactone-containing cyclic group represented by any of General Formulae (a2-r-1) to (a2-r-7), a —SO₂-containing cyclic group represented by any of General Formulae (b5-r-1) to (b5-r-4), and a heterocyclic group represented by any of Formulae (r-hr-7) to (r-hr-16).

Chain-like alkyl group which may have substituent:

The chain-like alkyl group as R¹⁰¹ may be linear or branched.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

The chain-like alkenyl group as R¹⁰¹ may be linear or branched, and the number of carbon atoms thereof is preferably in a range of 2 to 10, more preferably in a range of 2 to 5, still more preferably in a range of 2 to 4, and particularly preferably 3. Examples of the linear alkenyl group include a vinyl group, a propenyl group (an allyl group), and a butynyl group. Examples of the branched alkenyl group include a 1-methylvinyl group, a 2-methylvinyl group, a 1-methylpropenyl group, and a 2-methylpropenyl group.

Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R¹⁰¹ include an alkoxy group, a halogen atom, a halogenated alkyl group, a hydroxyl group, a carbonyl group, a nitro group, an amino group, and a cyclic group as R¹⁰¹.

Among the examples, R¹⁰¹ represents preferably a cyclic group which may have a substituent and more preferably a cyclic hydrocarbon group which may have a substituent.

More specifically, as the cyclic hydrocarbon group, a group in which one or more hydrogen atoms have been removed from a phenyl group, a naphthyl group, or a polycycloalkane; a lactone-containing cyclic group represented by any of General Formulae (a2-r-1) to (a2-r-7); or a —SO₂-containing cyclic group represented by any of General Formulae (b5-r-1) to (b5-r-4) is preferable, a group in which one or more hydrogen atoms have been removed from a polycycloalkane is more preferable, and an adamantyl group is still more preferable.

In Formula (b-1), Y¹⁰¹ represents a single bond or a divalent linking group having an oxygen atom.

In a case where Y¹⁰¹ represents a divalent linking group containing an oxygen atom, Y¹⁰¹ may contain an atom other than the oxygen atom. Examples of atoms other than an oxygen atom include a carbon atom, a hydrogen atom, a sulfur atom, and a nitrogen atom.

Examples of the divalent linking group having an oxygen atom include a non-hydrocarbon oxygen atom-containing linking group such as an oxygen atom (an ether bond: —O—), an ester bond (—C(═O)—O—), an oxycarbonyl group (—O—C(═O)—), an amide bond (—C(═O)—NH—), a carbonyl group (—C(═O)—), or a carbonate bond (—O—C(═O)—O—); and combinations of the above-described non-hydrocarbon oxygen atom-containing linking groups with an alkylene group. Further, a sulfonyl group (—SO₂—) may be further linked to the combination. Examples of the divalent linking group having an oxygen atom include linking groups each represented by General Formulae (y-a1-1) to (y-a1-7). Further, in General Formulae (y-a1-1) to (y-a1-7), V′¹⁰¹ in General Formulae (y-a1-1) to (y-a1-7) is bonded to R¹⁰¹ in Formula (b-1).

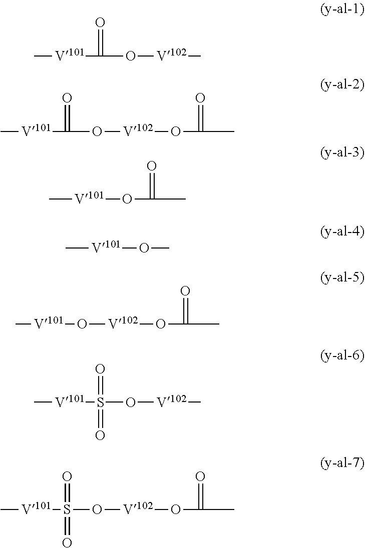

[In the formulae, V′¹⁰¹ represents a single bond or an alkylene group having 1 to 5 carbon atoms, and V′¹⁰² represents a divalent saturated hydrocarbon group having 1 to 30 carbon atoms.]

As the divalent saturated hydrocarbon group as V′¹⁰², an alkylene group having 1 to 30 carbon atoms is preferable, an alkylene group having 1 to 10 carbon atoms is more preferable, and an alkylene group having 1 to 5 carbon atoms is still more preferable.

The alkylene group as V′¹⁰¹ and V′¹⁰² may be a linear alkylene group or a branched alkylene group, and a linear alkylene group is preferable.

Specific examples of the alkylene group as V′¹⁰¹ and V′¹⁰² include a methylene group [—CH₂—]; an alkylmethylene group such as —CH(CH₃)—, —CH(CH₂CH₃)—, —C(CH₃)₂—, —C(CH₃)(CH₂CH₃)—, —C(CH₃)(CH₂CH₂CH₃)—, or —C(CH₂CH₃)₂—; an ethylene group [—CH₂CH₂—]; an alkylethylene group such as —CH(CH₃)CH₂—, —CH(CH₃)CH(CH₃)—, —C(CH₃)₂CH₂—, or —CH(CH₂CH₃)CH₂—; a trimethylene group (n-propylene group) [—CH₂CH₂CH₂—]; an alkyltrimethylene group such as —CH(CH₃)CH₂CH₂— or —CH₂CH(CH₃)CH₂—; a tetramethylene group [—CH₂CH₂CH₂CH₂—]; an alkyltetramethylene group such as —CH(CH₃)CH₂CH₂CH₂— or —CH₂CH(CH₃)CH₂CH₂—; and a pentamethylene group [—CH₂CH₂CH₂CH₂CH₂—].

Further, a part of the methylene group in the alkylene group as V′¹⁰¹ and V′¹⁰² may be substituted with a divalent aliphatic cyclic group having 5 to 10 carbon atoms. As the aliphatic cyclic group, a divalent group in which one hydrogen atom has been further removed from the cyclic aliphatic hydrocarbon group (a monocyclic aliphatic hydrocarbon group or a polycyclic aliphatic hydrocarbon group) as Ra′³ in Formula (a1-r-1) is preferable, and a cyclohexylene group, a 1,5-adamantylene group, or a 2,6-adamantylene group is more preferable.

Y¹⁰¹ represents preferably a divalent linking group having an ester bond or a divalent linking group having an ether bond and more preferably a linking group represented by any of Formulae (y-a1-1) to (y-a1-5).

In Formula (b-1), V¹⁰¹ (represents a single bond, an alkylene group, or a fluorinated alkylene group. It is preferable that the alkylene group and the fluorinated alkylene group as V¹⁰¹ have 1 to 4 carbon atoms. Examples of the fluorinated alkylene group as V¹⁰¹ include a group in which some or all hydrogen atoms in the alkylene group as V¹⁰¹ have been substituted with fluorine atoms. Among the examples, it is preferable that V¹⁰¹ represent a single bond or a linear fluorinated alkylene group having 1 to 4 carbon atoms.

In Formula (b-1), R¹⁰² represents a fluorine atom or a fluorinated alkyl group having 1 to 5 carbon atoms. R¹⁰² represents preferably a fluorine atom or a perfluoroalkyl group having 1 to 5 carbon atoms and more preferably a fluorine atom.

In a case where Y¹⁰¹ represents a single bond, specific examples of the anion moiety represented by Formula (b-1) include a fluorinated alkylsulfonate anion such as a trifluoromethanesulfonate anion or a perfluorobutanesulfonate anion. Further, in a case where Y¹⁰¹ represents a divalent linking group having an oxygen atom, specific examples thereof include an anion represented by any of Formulae (an-1) to (an-3).

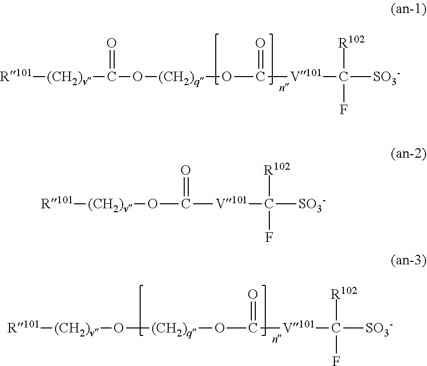

[In the formulae, R″¹⁰¹ represents an aliphatic cyclic group which may have a substituent, a monovalent heterocyclic group represented by any of Chemical Formulae (r-hr-1) to (r-hr-6), a condensed cyclic group represented by Formula (r-br-1) or (r-br-2), a chain-like alkyl group which may have a substituent, or an aromatic cyclic group which may have a substituent. R″¹⁰² represents an aliphatic cyclic group which may have a substituent, a condensed cyclic group represented by Formula (r-br-1) or (r-br-2), a lactone-containing cyclic group represented by any of General Formulae (a2-r-1) and (a2-r-3) to (a2-r-7), or a —SO₂-containing cyclic group represented by any of General Formulae (b5-r-1) to (b5-r-4). R″¹⁰³ represents an aromatic cyclic group which may have a substituent, an aliphatic cyclic group which may have a substituent, or a chain-like alkenyl group which may have a substituent. V″¹⁰¹ represents a single bond, an alkylene group having 1 to 4 carbon atoms, or a fluorinated alkylene group having 1 to 4 carbon atoms. R¹⁰² represents a fluorine atom or a fluorinated alkyl group having 1 to 5 carbon atoms. Each v″ independently represents an integer of 0 to 3, each q″ independently represents an integer of 0 to 20, and n″ represents 0 or 1.]

As the aliphatic cyclic group as R″¹⁰¹, R″¹⁰², and R″¹⁰³ which may have a substituent, the same groups as those for the cyclic aliphatic hydrocarbon group as R¹⁰¹ in Formula (b-1) are preferable. Examples of the substituent include the same groups as those for the substituent which may substitute the cyclic aliphatic hydrocarbon group as R¹⁰¹ in Formula (b-1).

The aromatic cyclic group which may have a substituent, as R″¹⁰¹ and R″¹⁰³, is preferably the group described as the example of the aromatic hydrocarbon group for the cyclic hydrocarbon group as R¹⁰¹ in General Formula (b-1). Examples of the substituent include the same groups as those for the substituent which may substitute the aromatic hydrocarbon group as R¹⁰¹ in Formula (b-1).

As the chain-like alkyl group as R″¹⁰¹ which may have a substituent, the same groups as those for the chain-like alkyl group as R¹⁰¹ in Formula (b-1) are preferable.

As the chain-like alkenyl group as R″¹⁰³ which may have a substituent, the same groups as those for the chain-like alkenyl group as R¹⁰¹ in Formula (b-1) are preferable.

The alkylene group and the fluorinated alkylene group as V″¹⁰¹ have preferably 1 to 3 carbon atoms and more preferably 1 or 2 carbon atoms. Examples of V″¹⁰¹ include —CH₂—, —(CH₂)—, —CFH—, —CH₂CFH—, and —CH(CF₃)—.

As the anion moiety represented by Formula (b-1), an anion moiety represented by Formula (an-1) is preferable. Among these, R″¹⁰¹ in Formula (an-1) represents preferably an aromatic ring group which may have a substituent and more preferably a phenyl group which may have a substituent. Examples of the substituent include a hydroxy group, an alkyl group, and a halogen atom. As the halogen atom, a bromine atom or an iodine atom is preferable, and an iodine atom is more preferable.

Anions in Component (b-2)

In Formula (b-2), R¹⁰⁴ and R¹⁰⁵ each independently represents a cyclic group which may have a substituent, a chain-like alkyl group which may have a substituent, or a chain-like alkenyl group which may have a substituent, and examples thereof include the same groups as those for R¹⁰¹ in Formula (b-1). Here, R¹⁰⁴ and R¹⁰⁵ may be bonded to each other to form a ring.

R¹⁰⁴ and R¹⁰⁵ represent preferably a chain-like alkyl group which may have a substituent and more preferably a linear or branched alkyl group or a linear or branched fluorinated alkyl group.

The chain-like alkyl group has preferably 1 to 10 carbon atoms, more preferably 1 to 7 carbon atoms, and still more preferably 1 to 3 carbon atoms. It is preferable that the number of carbon atoms in the chain-like alkyl group as R¹⁰⁴ and R¹⁰⁵ decrease within the range of the number of carbon atoms from the viewpoint that the solubility in a solvent for a resist is also satisfactory. Further, in the chain-like alkyl group as R¹⁰⁴ and R¹⁰⁵, it is preferable that the number of hydrogen atoms substituted with fluorine atoms be as large as possible from the viewpoint that the acid strength increases and the transparency to high energy light or electron beams having a wavelength of 250 nm or less is improved. The proportion of fluorine atoms in the chain-like alkyl group, that is, the fluorination ratio is preferably in a range of 70% to 100% and more preferably in a range of 90% to 100%, and it is most preferable that the chain-like alkyl group be a perfluoroalkyl group in which all hydrogen atoms are substituted with fluorine atoms.

In Formula (b-2), V¹⁰² and V¹⁰³ each independently represents a single bond, an alkylene group, or a fluorinated alkylene group, and examples thereof include the same groups as those for V¹⁰¹ in Formula (b-1).

In Formula (b-2), L¹⁰¹ and L¹⁰² each independently represents a single bond or an oxygen atom.

Anions in Component (b-3)

In Formula (b-3), R¹⁰⁶ to R¹⁰⁸ each independently represents a cyclic group which may have a substituent, a chain-like alkyl group which may have a substituent, or a chain-like alkenyl group which may have a substituent, and examples thereof include the same groups as those for R¹⁰¹ in Formula (b-1).

In Formula (b-3), L¹⁰³ to L¹⁰⁵ each independently represents a single bond, —CO—, or —SO₂—.

Among the examples, as the anion moiety of the component (B), an anion in the component (b-1) is preferable.

### {Cation Moiety}

In Formulae (b-1), (b-2), and (b-3), M′^{m+} represents an m-valent onium cation. Among these, a sulfonium cation and an iodonium cation are preferable.

m represents an integer of 1 or greater.

Preferred examples of the cation moiety ((M′^{m+})_{1/m}) include organic cations each represented by General Formulae (ca-1) to (ca-3). As the cation moiety, a cation represented by General Formula (ca-1) is more preferable, and a cation represented by any of Formulae (ca-1-1) to (ca-1-75) is still more preferable.

In the resist composition according to the present embodiment, the component (B) may be used alone or in combination of two or more kinds thereof.

The content of the component (B) in the resist composition is preferably in a range of 0 to 30 parts by mass, more preferably in a range of 0 to 20 parts by mass, still more preferably in a range of 0 to 10 parts by mass, and particularly preferably in a range of 0 to 5 parts by mass with respect to 100 parts by mass of the component (A).

It is preferable that the content of the component (B) be set to be in the above-described preferable ranges from the viewpoint that a uniform solution is likely to be obtained in a case where each component of the resist composition is dissolved in an organic solvent, and the storage stability of the resist composition is enhanced.

### <<Acid Diffusion Control Agent Component (D)>>

It is preferable that the resist composition according to the present embodiment further contain a base component (acid diffusion control agent component; hereinafter, also referred to as “component (D)”) that traps (that is, controls the acid diffusion) an acid that is generated upon light exposure, in addition to the component (A). The component (D) acts as a quencher (an acid diffusion control agent) which traps the acid generated in the resist composition upon light exposure.

Examples of the component (D) include a photodecomposable base (D1) having acid diffusion controllability (hereinafter, referred to as “component (D1)”) which is lost by the decomposition upon light exposure and a nitrogen-containing organic compound (D2) (hereinafter, referred to as “component (D2)”) which does not correspond to the component (D1). Among these, the photodecomposable base (the component (D1)) is preferable since it is easy to enhance the roughness reducing property. Further, in a case where the component (D1) is contained, both the characteristics of enhancing the sensitivity and suppressing the occurrence of coating defects are likely to be enhanced.

### In Regard to Component (D1)

In a case where a resist composition containing the component (D1) is obtained, the contrast between an exposed portion and an unexposed portion of the resist film can be further improved in a case of forming a resist pattern.

The component (D1) is not particularly limited as long as it is decomposed upon light exposure and loses acid diffusion controllability, and one or more compounds selected from the group consisting of a compound represented by General Formula (d1-1) (hereinafter, referred to as “component (d1-1)”), a compound represented by General Formula (d1-2) (hereinafter, referred to as “component (d1-2)”), and a compound represented by General Formula (d1-3) (hereinafter, referred to as “component (d1-3)”) are preferable.

Since the components (d1-1) to (d1-3) are decomposed and lose the acid diffusion controllability (basicity), they do not function as a quencher at the exposed portion of the resist film, but function as a quencher at the unexposed portion of the resist film.

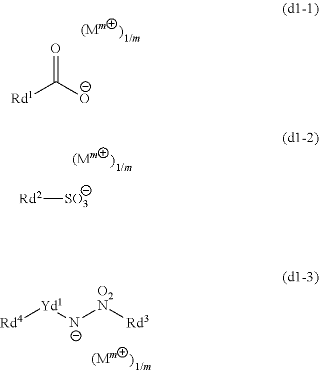

[In the formulae, Rd¹ to Rd⁴ represent a cyclic group which may have a substituent, a chain-like alkyl group which may have a substituent, or a chain-like alkenyl group which may have a substituent. Here, no fluorine atom is bonded to the carbon atom adjacent to the S atom in Rd² of Formula (d1-2). Yd¹ represents a single bond or a divalent linking group. m represents an integer of 1 or greater, and M^{m+}'s each independently represents an m-valent organic cation.]

{Component (d1-1)}

### Anion Moiety

In Formula (d1-1), Rd¹ represents a cyclic group which may have a substituent, a chain-like alkyl group which may have a substituent, or a chain-like alkenyl group which may have a substituent, and examples thereof include the same groups as those for R′²⁰¹.

Among these, it is preferable that Rd′ represent an aromatic hydrocarbon group which may have a substituent, an aliphatic cyclic group which may have a substituent, or a chain-like alkyl group which may have a substituent. Examples of the substituent that may be included in these groups include a hydroxyl group, an oxo group, an alkyl group, an aryl group, a fluorine atom, a fluorinated alkyl group, a lactone-containing cyclic group represented by any of General Formulae (a2-r-1) to (a2-r-7), an ether bond, an ester bond, and a combination thereof. In a case where an ether bond or an ester bond is included as the substituent, the substituent may be bonded through an alkylene group, and a linking group represented by any of Formulae (y-a1-1) to (y-a1-5) is preferable as the substituent. Further, in a case where the aromatic hydrocarbon group, the aliphatic cyclic group, or the chain-like alkyl group as Rd¹ contain a linking group represented by any of General Formulae (y-a1-1) to (y-a1-7) as a substituent, V′¹⁰¹ in General Formulae (y-a1-1) to (y-a1-7) is bonded to the carbon atom constituting the aromatic hydrocarbon group, the aliphatic cyclic group, or the chain-like alkyl group as Rd¹ in Formula (d3-1), in General Formulae (y-a1-1) to (y-a1-7).

Suitable examples of the aromatic hydrocarbon group include a phenyl group, a naphthyl group, and a polycyclic structure having a bicyclooctane skeleton (for example, a polycyclic structure formed of a bicyclooctane skeleton and a ring structure other than the bicyclooctane skeleton).

As the aliphatic cyclic group, a group in which one or more hydrogen atoms have been removed from a polycycloalkane such as adamantane, norbornane, isobornane, tricyclodecane or tetracyclododecane is more preferable.

It is preferable that the chain-like alkyl group have 1 to 10 carbon atoms, and specific examples thereof include a linear alkyl group such as a methyl group, an ethyl group, a propyl group, a butyl group, a pentyl group, a hexyl group, a heptyl group, an octyl group, a nonyl group, or a decyl group; and a branched alkyl group such as a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, or a 4-methylpentyl group.

In a case where the chain-like alkyl group is a fluorinated alkyl group having a fluorine atom or a fluorinated alkyl group as a substituent, the fluorinated alkyl group has preferably 1 to 11 carbon atoms, more preferably 1 to 8 carbon atoms, and still more preferably 1 to 4 carbon atoms. The fluorinated alkyl group may have an atom other than a fluorine atom. Examples of the atom other than a fluorine atom include an oxygen atom, a sulfur atom, and a nitrogen atom.

Specific preferred examples of the anion moiety in the component (d1-1) are described below.

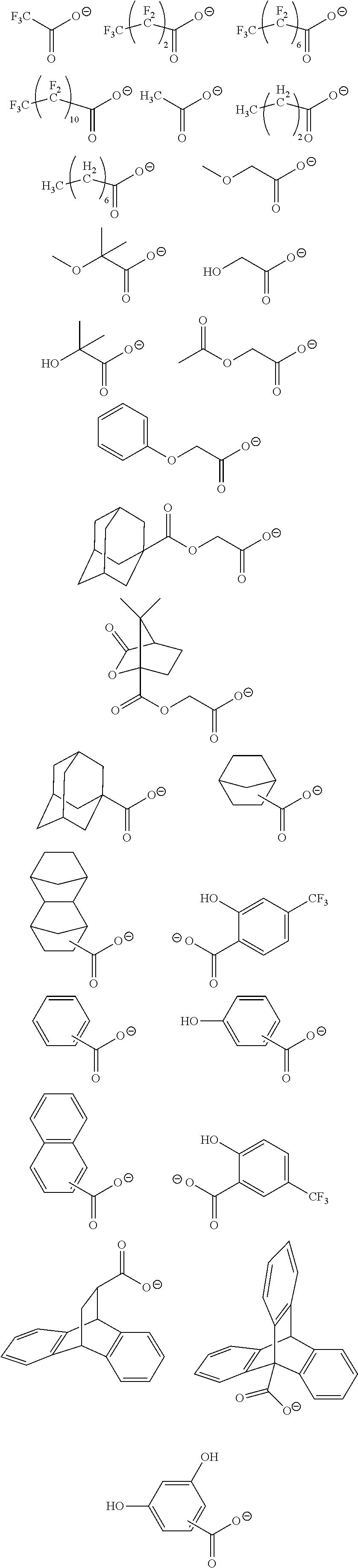

### Cation Moiety

In Formula (d1-1), M^{m+} represents an m-valent organic cation.

Suitable examples of the organic cation as M^{m+} include the same as those each represented by General Formulae (ca-1) to (ca-3). Among these, a cation represented by General Formula (ca-1) is more preferable, and a cation represented by any of Formulae (ca-1-1) to (ca-1-75) is still more preferable.

The component (d1-1) may be used alone or in combination of two or more kinds thereof.

{Component (d1-2)}

### Anion Moiety

In Formula (d1-2), Rd² represents a cyclic group which may have a substituent, a chain-like alkyl group which may have a substituent, or a chain-like alkenyl group which may have a substituent, and examples thereof include the same groups as those for R′²⁰¹.

Here, no fluorine atom is bonded to the carbon atom adjacent to the S atom in Rd² (the carbon atom is not substituted with fluorine). In this manner, the anion of the component (d1-2) is an appropriately weak acid anion, thereby improving the quenching ability of the component (D).

Rd² represents preferably a chain-like alkyl group which may have a substituent or an aliphatic cyclic group which may have a substituent and more preferably an aliphatic cyclic group which may have a substituent.

The chain-like alkyl group preferably has 1 to 10 carbon atoms and more preferably 3 to 10 carbon atoms.

The aliphatic cyclic group is more preferably a group (which may have a substituent) obtained by removing one or more hydrogen atoms from adamantane, norbornane, isobornane, tricyclodecane, tetracyclododecane, or the like; and a group obtained by removing one or more hydrogen atoms from camphor.

The hydrocarbon group as Rd² may have a substituent, and examples of the substituent include the same groups as those for the substituent that the hydrocarbon group (such as an aromatic hydrocarbon group, an aliphatic cyclic group, or a chain-like alkyl group) as Rd¹ in Formula (d1-1) may have.

Specific preferred examples of the anion moiety in the component (d1-2) are described below.

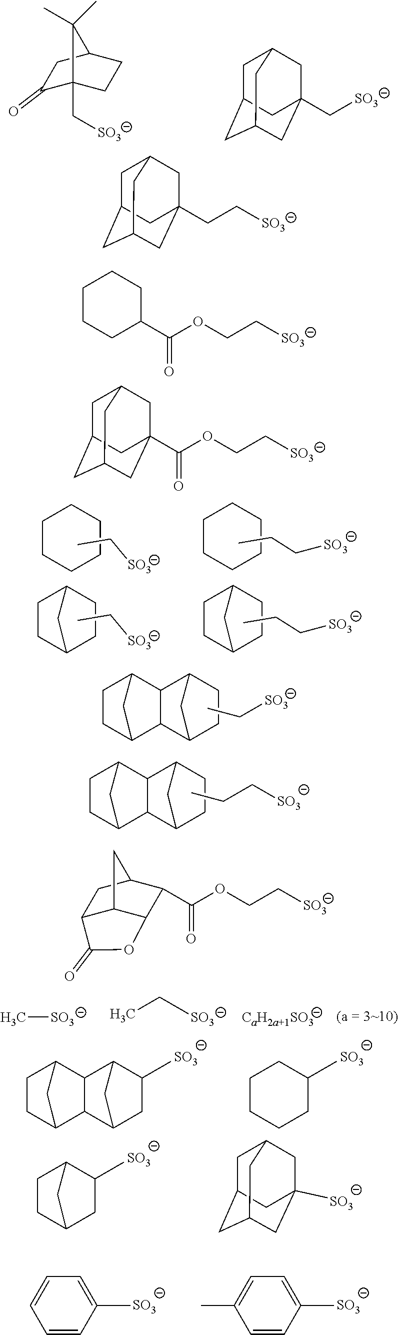

### Cation Moiety

In Formula (d1-2), M^{m+} represents an m-valent organic cation and has the same definition as that for M^{m+} in Formula (d1-1).

The component (d1-2) may be used alone or in combination of two or more kinds thereof.

{Component (d1-3)}

### Anion Moiety

In Formula (d1-3), Rd³ represents a cyclic group which may have a substituent, a chain-like alkyl group which may have a substituent, or a chain-like alkenyl group which may have a substituent, and examples thereof include the same groups as those for R′²⁰¹. Among these, a cyclic group having a fluorine atom, a chain-like alkyl group, or a chain-like alkenyl group is preferable. Among these, a fluorinated alkyl group is preferable, and the same groups as those for the fluorinated alkyl group represented by Rd¹ are more preferable.

In Formula (d1-3), Rd⁴ represents a cyclic group which may have a substituent, a chain-like alkyl group which may have a substituent, or a chain-like alkenyl group which may have a substituent, and examples thereof include the same groups as those for R′²⁰¹.

Among these, an alkyl group which may have a substituent, an alkoxy group which may have a substituent, an alkenyl group which may have a substituent, or a cyclic group which may have a substituent is preferable.

It is preferable that the alkyl group as Rd⁴ be a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. Some hydrogen atoms in the alkyl group as Rd⁴ may be substituted with a hydroxyl group, a cyano group, or the like.

It is preferable that the alkoxy group as Rd⁴ be an alkoxy group having 1 to 5 carbon atoms, and specific examples of the alkoxy group having 1 to 5 carbon atoms include a methoxy group, an ethoxy group, an n-propoxy group, an iso-propoxy group, an n-butoxy group, and a tert-butoxy group. Among these, a methoxy group and an ethoxy group are preferable.

Examples of the alkenyl group as Rd⁴ include the same groups as those for the alkenyl group as R′²⁰¹. Among these, a vinyl group, a propenyl group (an allyl group), a 1-methylpropenyl group, and a 2-methylpropenyl group are preferable. These groups may have an alkyl group having 1 to 5 carbon atoms or a halogenated alkyl group having 1 to 5 carbon atoms as a substituent.

Examples of the cyclic group as Rd⁴ include the same groups as those for the cyclic group as R′²⁰¹. Among these, an alicyclic group in which one or more hydrogen atoms have been removed from a cycloalkane such as cyclopentane, cyclohexane, adamantane, norbornane, isobornane, tricyclodecane or tetracyclododecane or an aromatic group such as a phenyl group or a naphthyl group is preferable. In a case where Rd⁴ represents an alicyclic group, the resist composition is satisfactorily dissolved in an organic solvent so that the lithography characteristics are enhanced. Further, in a case where Rd⁴ represents an aromatic group, the resist composition has excellent light absorption efficiency in lithography using EUV or the like as an exposure light source, and thus the sensitivity and lithography characteristics are enhanced.

In Formula (d1-3), Yd¹ represents a single bond or a divalent linking group.

The divalent linking group as Yd¹ is not particularly limited, and examples thereof include a divalent hydrocarbon group (an aliphatic hydrocarbon group or an aromatic hydrocarbon group) which may have a substituent and a divalent linking group having a heteroatom. Examples of the divalent linking groups are the same as those for the divalent hydrocarbon group which may have a substituent and the divalent linking group having a heteroatom described in the section of the divalent linking group as Ya²¹ in Formula (a2-1).

It is preferable that Yd¹ represent a carbonyl group, an ester bond, an amide bond, an alkylene group, or a combination thereof. As the alkylene group, a linear or branched alkylene group is more preferable, and a methylene group or an ethylene group is still more preferable.

Specific preferred examples of the anion moiety in the component (d1-3) are described below.

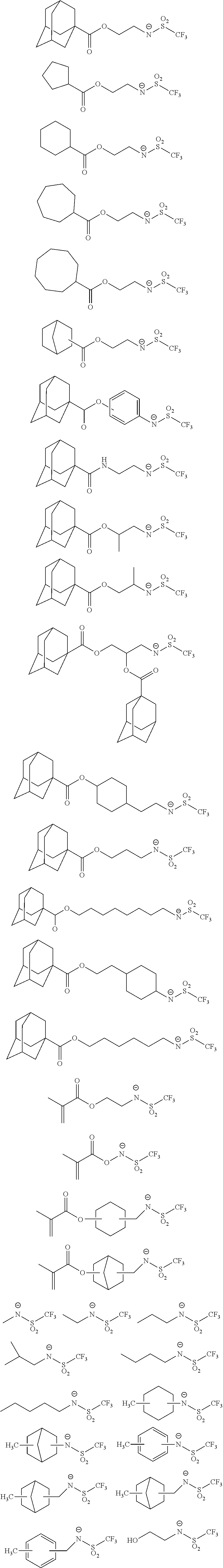

### Cation Moiety

In Formula (d1-3), M^{m+} represents an m-valent organic cation and has the same definition as that for M^{m+} in Formula (d1-1).

The component (d1-3) may be used alone or in combination of two or more kinds thereof.

As the component (D1), only one of the above-described components (d1-1) to (d1-3) or a combination of two or more kinds thereof may be used.

In a case where the resist composition contains the component (D1), the content of the component (D1) in the resist composition is preferably in a range of 0.5 to 15 parts by mass, more preferably in a range of 1 to 10 parts by mass, and still more preferably in a range of 2 to 8 parts by mass with respect to 100 parts by mass of the component (A).

In a case where the content of the component (D1) is greater than or equal to the lower limits of the above-described preferable ranges, satisfactory lithography characteristics and a satisfactory resist pattern shape are likely to be obtained. On the contrary, in a case where the content is less than or equal to the upper limits of the above-described ranges, the sensitivity can be satisfactorily maintained and the throughput is also excellent.

In the resist composition according to the present embodiment, it is preferable that the component (D1) contain the component (d1-1).

The content of the component (d1-1) in the total component (D) contained in the resist composition according to the present embodiment is preferably 50% by mass or greater, preferably 70% by mass or greater, and still more preferably 90% by mass or greater, and the component (D) may consist of only the component (d1-1).

### Method of Producing Component (D1):

The methods of producing the component (d1-1) and the component (d1-2) are not particularly limited, and these components can be produced by known methods.

Further, the method of producing the component (d1-3) is not particularly limited, and the component is produced by the same method as disclosed in United States Patent Application, Publication No. 2012-0149916.

### In Regard to Component (D2)

The component (D) may contain a nitrogen-containing organic compound component (hereinafter, referred to as “component (D2)”) that does not correspond to the component (D1) described above.

The component (D2) is not particularly limited as long as it functions as an acid diffusion control agent and does not correspond to the component (D1), and an optional component may be selected from known components and then used. Among the examples, an aliphatic amine is preferable, and particularly a secondary aliphatic amine and a tertiary aliphatic amine are more preferable.

The aliphatic amine is an amine containing one or more aliphatic groups, and the number of carbon atoms in the aliphatic group is preferably in a range of 1 to 12.

Examples of the aliphatic amine include amines in which at least one hydrogen atom of ammonia NH₃ has been substituted with an alkyl group or hydroxyalkyl group having 12 or less carbon atoms (alkylamines or alkylalcoholamines), and cyclic amines.

Specific examples of the alkylamines and the alkylalcoholamines include monoalkylamines such as n-hexylamine, n-heptylamine, n-octylamine, n-nonylamine, and n-decylamine; dialkylamines such as diethylamine, di-n-propylamine, di-n-heptylamine, di-n-octylamine, and dicyclohexylamine; trialkylamines such as trimethylamine, triethylamine, tri-n-propylamine, tri-n-butylamine, tri-n-pentylamine, tri-n-hexylamine, tri-n-heptylamine, tri-n-octylamine, tri-n-nonylamine, tri-n-decylamine, and tri-n-dodecylamine; and alkylalcoholamines such as diethanolamine, triethanolamine, diisopropanolamine, triisopropanolamine, di-n-octanolamine, and tri-n-octanolamine. Among these, a trialkylamine having 6 to 30 carbon atoms is still more preferable, and tri-n-pentylamine or tri-n-octylamine is particularly preferable.

Examples of the cyclic amine include a heterocyclic compound having a nitrogen atom as a heteroatom. The heterocyclic compound may be a monocyclic compound (aliphatic monocyclic amine) or a polycyclic compound (aliphatic polycyclic amine).

Specific examples of the aliphatic monocyclic amine include piperidine and piperazine.

The aliphatic polycyclic amine preferably has 6 to 10 carbon atoms, and specific examples thereof include 1,5-diazabicyclo[4.3.0]-5-nonene, 1,8-diazabicyclo[5.4.0]-7-undecene, hexamethylenetetramine, and 1,4-diazabicyclo[2.2.2]octane.

Examples of other aliphatic amines include tris(2-methoxymethoxyethyl)amine, tris{2-(2-methoxyethoxy)ethyl}amine, tris{2-(2-methoxyethoxymethoxy)ethyl}amine, tris{2-(1-methoxyethoxy)ethyl}amine, tris{2-(1-ethoxyethoxy)ethyl}amine, tris{2-(1-ethoxypropoxy)ethyl}amine, tris[2-{2-(2-hydroxyethoxy)ethoxy}ethyl]amine, and triethanolamine triacetate. Among these, triethanolamine triacetate is preferable.

As the component (D2), an aromatic amine may be used.

Examples of aromatic amines include 4-dimethylaminopyridine, pyrrole, indole, pyrazole, imidazole, and derivatives thereof, tribenzylamine, 2,6-diisopropylaniline, N-tert-butoxycarbonylpyrrolidine, and 2,6-di-tert-butylpyridine.

Among the examples, the component (D2) is preferably an alkylamine and more preferably a trialkylamine having 5 to 10 carbon atoms.

The component (D2) may be used alone or in combination of two or more kinds thereof.

In a case where the resist composition contains the component (D2), the content thereof in the resist composition is preferably in a range of 0.01 to 5 parts by mass, more preferably in a range of 0.1 to 5 parts by mass, and still more preferably in a range of 0.5 to 5 parts by mass with respect to 100 parts by mass of the component (A).

In a case where the content of the component (D2) is greater than or equal to the lower limits or the above-described preferable ranges, excellent lithography characteristics and an excellent resist pattern shape are easily obtained. On the contrary, in a case where the content is less than or equal to the upper limits of the above-described ranges, the sensitivity can be satisfactorily maintained and the throughput is also excellent.

<<At Least One Compound (E) Selected from Group Consisting of Organic Carboxylic Acids, Phosphorus Oxo Acids, and Derivatives Thereof>>

For the purpose of preventing any deterioration in sensitivity and improving the resist pattern shape and the post-exposure temporal stability, the resist composition according to the present embodiment may contain, as an optional component, at least one compound (E) (hereinafter referred to as “component (E)”) selected from the group consisting of an organic carboxylic acid, and a phosphorus oxo acid and a derivative thereof.

Specific examples of the organic carboxylic acid include acetic acid, malonic acid, citric acid, malic acid, succinic acid, benzoic acid, and salicylic acid. Among these, salicylic acid is preferable.

Examples of the phosphorus oxo acid include phosphoric acid, phosphonic acid, and phosphinic acid. Among these, phosphonic acid is particularly preferable.

In the resist composition of the present embodiment, the component (E) may be used alone or in combination of two or more kinds thereof.

In a case where the resist composition contains the component (E), the content of the component (E) is preferably in a range of 0.01 to 5 parts by mass and more preferably in a range of 0.05 to 3 parts by mass with respect to 100 parts by mass of the component (A). In a case where the content thereof is in the above-described ranges, the lithography characteristics are further improved.

### <<Fluorine Additive Component (F)>>

The resist composition according to the present embodiment may further contain a fluorine additive component (hereinafter, referred to as “component (F)”) as a hydrophobic resin. The component (F) is used to impart water repellency to the resist film and used as a resin different from the component (A), and thus the lithography characteristics are enhanced.

As the component (F), for example, the fluorine-containing polymer compounds described in Japanese Unexamined Patent Application, First Publication Nos. 2010-002870, 2010-032994, 2010-277043, 2011-13569, and 2011-128226 can be used.

Specific examples of the component (F) include a polymer having a constitutional unit (f1) represented by General Formula (f1-1). As the polymer, a polymer (homopolymer) formed of only the constitutional unit (f1) represented by Formula (f1-1); a copolymer of the constitutional unit (f1) and the constitutional unit (a1); or a copolymer of the constitutional unit (f1), a constitutional unit derived from acrylic acid or methacrylic acid, and the constitutional unit (a1) is preferable, and a copolymer of the constitutional unit (f1) and the constitutional unit (a1) is more preferable. Here, as the constitutional unit (a1) copolymerized with the constitutional unit (f1), a constitutional unit derived from 1-ethyl-1-cyclooctyl (meth)acrylate or a constitutional unit derived from 1-methyl-1-adamantyl (meth)acrylate is preferable, and a constitutional unit derived from 1-ethyl-1-cyclooctyl (meth)acrylate is more preferable.

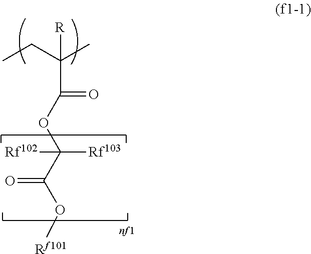

[In the formula, R has the same definition as described above, Rf¹⁰² and Rf¹⁰³ each independently represents a hydrogen atom, a halogen atom, an alkyl group having 1 to 5 carbon atoms, or a halogenated alkyl group having 1 to 5 carbon atoms, and Rf¹⁰² and Rf¹⁰³ may be the same as or different from each other. nf¹ represents an integer of 0 to 5, and Rf¹⁰¹ represents an organic group having a fluorine atom.]

In Formula (f1-1), R bonded to the carbon atom at the α-position has the same definition as described above. It is preferable that R represent a hydrogen atom or a methyl group.

In Formula (f1-1), a fluorine atom is preferable as the halogen atom as Rf¹⁰² and Rf¹⁰³. Examples of the alkyl group having 1 to 5 carbon atoms as Rf¹⁰² and Rf¹⁰³ include the same groups as those for the alkyl group having 1 to 5 carbon atoms as R. Among the examples, a methyl group or an ethyl group is preferable. Specific examples of the halogenated alkyl group having 1 to 5 carbon atoms as Rf¹⁰² and Rf¹⁰³ include groups in which some or all hydrogen atoms of an alkyl group having 1 to 5 carbon atoms are substituted with halogen atoms. Among these, a fluorine atom is preferable as the halogen atom. Among these, Rf¹⁰² and Rf¹⁰³ represent preferably a hydrogen atom, a fluorine atom, or an alkyl group having 1 to 5 carbon atoms, more preferably a hydrogen atom, a fluorine atom, a methyl group, or an ethyl group, and still more preferably a hydrogen atom.

In Formula (f1-1), nf¹ represents an integer of 0 to 5, preferably an integer of 0 to 3, and more preferably 1 or 2.

In Formula (f1-1), Rf¹⁰¹ represents an organic group having a fluorine atom and preferably a hydrocarbon group having a fluorine atom.

The hydrocarbon group having a fluorine atom may be linear, branched, or cyclic, and the number of carbon atoms thereof is preferably in a range of 1 to 20, more preferably in a range of 1 to 15, and particularly preferably in a range of 1 to 10.

In the hydrocarbon group having a fluorine atom, preferably 25% or more of the hydrogen atoms in the hydrocarbon group are fluorinated, more preferably 50% or more thereof are fluorinated, and particularly preferably 60% or more thereof are fluorinated from the viewpoint of increasing the hydrophobicity of the resist film during immersion exposure.

Among the examples, Rf¹⁰¹ represents more preferably a fluorinated hydrocarbon group having 1 to 6 carbon atoms and particularly preferably a trifluoromethyl group, —CH₂—CF₃, —CH₂—CF₂—CF₃, —CH(CF₃)₂, —CH₂—CH₂—CF₃, or —CH₂—CH₂—CF₂—CF₂—CF₂—CF₃.

The weight-average molecular weight (Mw) (in terms of polystyrene according to gel permeation chromatography) of the component (F) is preferably in a range of 1000 to 50000, more preferably in a range of 5000 to 40000, and most preferably in a range of 10000 to 30000. In a case where the weight-average molecular weight thereof is less than or equal to the upper limits of the above-described ranges, the resist composition exhibits sufficiently satisfactory solubility in a resist solvent to be used as a resist. Meanwhile, in a case where the weight-average molecular weight thereof is greater than or equal to the lower limits of the above-described ranges, water repellency of the resist film is improved.

Further, the dispersity (Mw/Mn) of the component (F) is preferably in a range of 1.0 to 5.0, more preferably in a range of 1.0 to 3.0, and most preferably in a range of 1.0 to 2.5.

In the resist composition according to the present embodiment, the component (F) may be used alone or in combination of two or more kinds thereof.

In a case where the resist composition contains the component (F), the content of the component (F) is preferably in a range of 0.5 to 10 parts by mass and more preferably in a range of 1 to 10 parts by mass with respect to 100 parts by mass of the component (A).

### <<Organic Solvent Component(S)>>

The resist composition of the present embodiment can be produced by dissolving the resist materials in an organic solvent component (hereinafter, referred to as “component(S)”).

The component(S) may be any organic solvent which can dissolve each component used to obtain a uniform solution, and an optional organic solvent can be appropriately selected from those known as solvents of a chemically amplified resist composition and then used.

Examples of the component(S) include lactones such as γ-butyrolactone; ketones such as acetone, methyl ethyl ketone, cyclohexanone, methyl-n-pentyl ketone, methyl isopentyl ketone, and 2-heptanone; polyhydric alcohols such as ethylene glycol, diethylene glycol, propylene glycol, and dipropylene glycol; derivatives of polyhydric alcohols such as compounds having an ester bond, for example, ethylene glycol monoacetate, diethylene glycol monoacetate, propylene glycol monoacetate, and dipropylene glycol monoacetate, and compounds having an ether bond, for example, monoalkyl ether and monophenyl ether such as monomethyl ether, monoethyl ether, monopropyl ether, and monobutyl ether of the polyhydric alcohols or the compound having an ester bond; cyclic ethers such as dioxane; esters such as methyl lactate, ethyl lactate (EL), methyl acetate, ethyl acetate, butyl acetate, methyl pyruvate, ethyl pyruvate, methyl methoxypropionate, and ethyl ethoxypropionate; aromatic organic solvents such as anisole, ethyl benzyl ether, cresyl methyl ether, diphenyl ether, dibenzyl ether, phenetole, butyl phenyl ether, ethylbenzene, diethylbenzene, pentylbenzene, isopropylbenzene, toluene, xylene, cymene, and mesitylene, and dimethyl sulfoxide (DMSO).

In the resist composition of the present embodiment, the component(S) may be used alone or in the form of a mixed solvent of two or more kinds thereof. Among these, PGMEA, PGME, γ-butyrolactone, EL, or cyclohexanone is preferable.

Further, a mixed solvent obtained by mixing PGMEA with a polar solvent is also preferable as the component(S). The blending ratio (mass ratio) of the mixed solvent can be appropriately determined in consideration of the compatibility between PGMEA and the polar solvent, but is preferably in the range of 1:9 to 9:1 and more preferably in a range of 2:8 to 8:2.

More specifically, in a case where EL or cyclohexanone is blended as the polar solvent, the mass ratio of PGMEA to EL or cyclohexanone is preferably in a range of 1:9 to 9:1 and more preferably in a range of 2:8 to 8:2. Further, in a case where PGME is blended as the polar solvent, the mass ratio of PGMEA to PGME is preferably in a range of 1:9 to 9:1, more preferably in a range of 2:8 to 8:2, and still more preferably in a range of 3:7 to 7:3. Further, a mixed solvent of PGMEA, PGME, and cyclohexanone is also preferable.

A mixed solvent of at least one selected from PGMEA and EL, and γ-butyrolactone is also preferable as the(S) component. In this case, as the mixing ratio, the mass ratio between the former and the latter is preferably in a range of 70:30 to 95:5.

The amount of the component(S) to be used is not particularly limited and is appropriately set to have a concentration which enables coating a substrate or the like depending on the thickness of the coated film. The component(S) is typically used in an amount such that the solid content concentration of the resist composition is set to be in a range of 0.1% to 20% by mass and preferably in a range of 0.2% to 15% by mass.

After the resist material is dissolved in the component(S), impurities may be removed from the resist composition of the present embodiment using a porous polyimide film, a porous polyamideimide film, or the like. For example, the resist composition may be filtered using a filter formed of a porous polyimide film, a filter formed of a porous polyamideimide film, a filter formed of a porous polyimide film and a porous polyamideimide film, or the like. Examples of the porous polyimide film and the porous polyamideimide film include those described in Japanese Unexamined Patent Application, First Publication No. 2016-155121.

The resist composition according to the present embodiment described above contains the resin component (A1) having the constitutional unit (a0). In this manner, high sensitivity can be achieved while a satisfactory pattern shape is maintained. In addition, the roughness and defects can be reduced.

The reason such an effect is exhibited is assumed as follows.

Since the resin component (A1) has the constitutional unit (a0) containing an acid generator component, extreme diffusion of the acid generated upon light exposure is suppressed. Therefore, more acid generator components can be introduced, and the change in solubility in a developing solution due to light exposure can be increased. In this manner, high sensitivity can be achieved, and the roughness can be reduced. Further, the acid generated from the constitutional unit (a0) has three fluorine atoms, and thus the hydrophilicity and the hydrophobicity can be balanced. Therefore, it is considered that the distribution of the constitutional unit (a0) in the entire resist film is uniform, and the deprotection in the exposed portion proceeds uniformly. In this manner, a pattern having a satisfactory shape with high rectangularity can be formed. In addition, it is assumed that defects derived from pattern breakage or residues can be suppressed because the fluorine atoms are appropriately disposed around the anion. It is considered that the roughness resistance is also improved.

### (Resist Pattern Formation Method)

A resist pattern formation method according to the second aspect of the present invention is a method including a step of forming a resist film on a support using the resist composition according to the first aspect of the present invention described above, a step of exposing the resist film to light, and a step of developing the resist film exposed to light to form a resist pattern.

According to the embodiment of the resist pattern formation method, a resist pattern formation method by performing processes as described below is an exemplary example.

First, a support is coated with the resist composition of the above-described embodiment using a spinner or the like, and a bake (post-applied bake (PAB)) treatment is performed under a temperature condition of 80° C. to 150° C. for 40 to 120 seconds and preferably 60 to 90 seconds to form a resist film.

Following the selective exposure carried out on the resist film by, for example, exposure through a mask (mask pattern) having a predetermined pattern formed on the mask using an exposure apparatus such as an electron beam lithography apparatus or an ArF exposure apparatus, or direct irradiation of the resist film for drawing with an electron beam without using a mask pattern, a bake treatment (post-exposure bake (PEB)) is carried out, for example, under a temperature condition in a range of 80° C. to 150° C. for 40 to 120 seconds and preferably 60 to 90 seconds.

Next, the resist film is subjected to a developing treatment. The developing treatment is conducted using an alkali developing solution in a case of an alkali developing process and using a developing solution containing an organic solvent (organic developing solution) in a case of a solvent developing process.

After the developing treatment, it is preferable to conduct a rinse treatment. As the rinse treatment, water rinsing using pure water is preferable in a case of the alkali developing process, and rinsing using a rinse solution containing an organic solvent is preferable in a case of the solvent developing process.

In a case of the solvent developing process, after the developing treatment or the rinse treatment, the developing solution or the rinse solution attached onto the pattern may be removed by a treatment using a supercritical fluid.

After the developing treatment or the rinse treatment, drying is conducted. As desired, a bake treatment (post bake) may be conducted after the developing treatment.

In this manner, a resist pattern can be formed.

The support is not particularly limited and a known support of the related art can be used, and examples thereof include a substrate for an electronic component and a substrate on which a predetermined wiring pattern has been formed. Specific examples thereof include a metal substrate such as a silicon wafer, copper, chromium, iron, or aluminum; and a glass substrate. As the materials of the wiring pattern, copper, aluminum, nickel, or gold can be used.

The wavelength used for light exposure is not particularly limited and the exposure can be conducted using radiation such as an ArF excimer laser, a KrF excimer laser, an F₂ excimer laser, extreme ultraviolet rays (EUV), vacuum ultraviolet rays (VUV), electron beams (EB), X-rays, and soft X-rays. The resist composition is useful for a KrF excimer laser, an ArF excimer laser, EB, and BUV, more useful for an ArF excimer laser, EB, and EUV, and particularly useful for EB and EUV. That is, the resist pattern formation method according to the present embodiment is a method particularly useful in a case where the step of exposing the resist film to light includes a process of exposing the resist film to extreme ultraviolet (EUV) rays or electron beams (EB).

The method for exposing the resist film to light can be a general exposure (dry exposure) conducted in air or an inert gas such as nitrogen, or liquid immersion exposure (liquid immersion lithography).

The liquid immersion exposure is an exposure method in which the region between the resist film and the lens at the lowermost position of the exposure apparatus is filled with a solvent (liquid immersion medium) in advance that has a refractive index greater than the refractive index of air, and the exposure (immersion exposure) is conducted in this state.

As the liquid immersion medium, a solvent having a refractive index greater than the refractive index of air but less than the refractive index of the resist film to be exposed is preferable, and examples thereof include water, a fluorine-based inert liquid, a silicon-based solvent, and a hydrocarbon-based solvent.

As the liquid immersion medium, water is preferably used.

As the alkali developing solution used for the developing treatment in the alkali developing process, a 0.1 to 10 mass % tetramethylammonium hydroxide (TMAH) aqueous solution is an exemplary example.

The organic solvent contained in the organic developing solution used for the developing treatment in the solvent developing process may be any solvent that is capable of dissolving the component (A) (the component (A) before light exposure) and can be appropriately selected from known organic solvents. Specific examples thereof include a polar solvent such as a ketone-based solvent, an ester-based solvent, an alcohol-based solvent, a nitrile-based solvent, an amide-based solvent, and an ether-based solvent, and a hydrocarbon-based solvent.

Examples of the ester-based solvent include methyl acetate, butyl acetate, ethyl acetate, isopropyl acetate, pentyl acetate, isopentyl acetate, amyl acetate, propylene glycol monomethyl ether acetate, ethylene glycol monoethyl ether acetate, diethylene glycol monobutyl ether acetate, diethylene glycol monoethyl ether acetate, ethyl-3-ethoxypropionate, 3-methoxybutyl acetate, 3-methyl-3-methoxybutyl acetate, methyl formate, ethyl formate, butyl formate, propyl formate, ethyl lactate, butyl lactate, propyl lactate, butyl butanoate, methyl 2-hydroxyisobutyrate, isoamyl acetate, isobutyl isobutyrate, and butyl propionate.

Examples of the nitrile-based solvent include acetonitrile, propionitrile, valeronitrile, and butyronitrile.

Known additives can be blended into the organic developing solution as necessary. Examples of the additive include a surfactant. The surfactant is not particularly limited, and for example, an ionic or non-ionic fluorine-based and/or silicon-based surfactant can be used. As the surfactant, a non-ionic surfactant is preferable, and a non-ionic fluorine-based surfactant or a non-ionic silicon-based surfactant is more preferable.

In a case where a surfactant is blended into the solution, the amount of the surfactant to be blended is typically in a range of 0.001% to 5% by mass, preferably in a range of 0.005% to 2% by mass, and more preferably in a range of 0.01% to 0.5% by mass with respect to the total amount of the organic developing solution.

The developing treatment can be performed according to a known developing method, and examples thereof include a method of immersing a support in a developing solution for a certain time (a dip method), a method of raising a developing solution on the surface of a support using the surface tension and maintaining the state for a certain time (a puddle method), a method of spraying a developing solution to the surface of a support (spray method), and a method of continuously ejecting a developing solution onto a support rotating at a certain rate while scanning a developing solution ejection nozzle at a certain rate (dynamic dispense method).

As the organic solvent contained in the rinse solution used for the rinse treatment after the developing treatment in the solvent developing process, a solvent that is unlikely to dissolve a resist pattern can be appropriately selected from the organic solvents described as the organic solvent used in the organic developing solution and then used. Typically, at least one solvent selected from a hydrocarbon-based solvent, a ketone-based solvent, an ester-based solvent, an alcohol-based solvent, an amide-based solvent, and an ether-based solvent is used. Among these, at least one solvent selected from a hydrocarbon-based solvent, a ketone-based solvent, an ester-based solvent, an alcohol-based solvent, and an amide-based solvent is preferable, at least one solvent selected from an alcohol-based solvent and an ester-based solvent is more preferable, and an alcohol-based solvent is particularly preferable.

As the alcohol-based solvent used in the rinse solution, a monohydric alcohol having 6 to 8 carbon atoms is preferable, and the monohydric alcohol may be linear, branched, or cyclic. Specific examples thereof include 1-hexanol, 1-heptanol, 1-octanol, 2-hexanol, 2-heptanol, 2-octanol, 3-hexanol, 3-heptanol, 3-octanol, 4-octanol, and benzyl alcohol. Among these, 1-hexanol, 2-heptanol, and 2-hexanol are preferable, and 1-hexanol and 2-hexanol are more preferable.

These organic solvents may be used alone or in combination of two or more kinds thereof. Further, an organic solvent other than the above-described solvents and water may be mixed and used. However, in consideration of the development characteristics, the amount of water to be blended into the rinse solution is preferably 30% by mass or less, more preferably 10% by mass or less, still more preferably 5% by mass or less, and particularly preferably 3% by mass or less with respect to the total amount of the rinse solution.

A known additive can be blended into the rinse solution as necessary. Examples of the additive include a surfactant. As the surfactant, the same surfactants as those described above are exemplary examples. Among these, a non-ionic surfactant is preferable, and a non-ionic fluorine-based surfactant or a non-ionic silicon-based surfactant is more preferable.

In a case where a surfactant is blended into the solution, the amount of the surfactant to be blended is typically in a range of 0.001% to 5% by mass, preferably in a range of 0.005% to 2% by mass, and more preferably in a range of 0.01% to 0.5% by mass with respect to the total amount of the rinse solution.

The rinse treatment carried out using a rinse solution (washing treatment) can be performed according to a known rinse method. Examples of the method of performing the rinse treatment include a method pf continuously ejecting a rinse solution onto a support rotating at a certain rate (rotary coating method), a method of immersing a support in a rinse solution for a certain time (dip method), and a method of spraying a rinse solution to the surface of a support (spray method).

According to the resist pattern formation method of the present embodiment described above, since the resist composition described above is used, a resist pattern in which high sensitivity is achieved and the roughness reducing property is satisfactory can be formed.

It is preferable that various materials that are used in the resist composition according to the above-described embodiment and the pattern forming method according to the above-described embodiment (for example, a resist solvent, a developing solution, a rinse solution, a composition for forming an antireflection film, and a composition for forming a top coat) not contain impurities such as a metal, a metal salt containing halogen, an acid, an alkali, and a component having a sulfur atom or phosphorus atom. Here, examples of the impurities containing metal atoms include Na, K, Ca, Fe, Cu, Mn, Mg, Al, Cr, Ni, Zn, Ag, Sn, Pb, Li, and salts thereof. The content of the impurities contained in these materials is preferably 200 ppb or less, more preferably 1 ppb or less, still more preferably 100 parts per trillion (ppt) or less, particularly preferably 10 ppt or less, and most preferably substantially zero (less than or equal to the detection limit of the measuring device).

### (Compound)

A compound according to a third aspect of the present invention is a compound represented by General Formula (m0-1) (hereinafter, also referred to as “compound (M0)”).

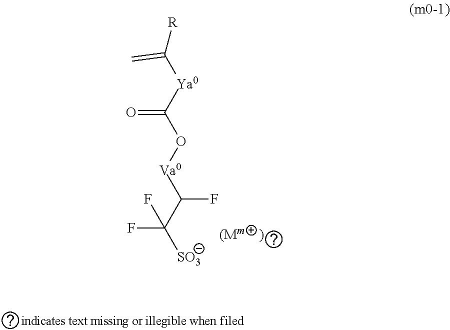

[In the formula, R represents a hydrogen atom, an alkyl group having 1 to 5 carbon atoms, a halogen atom, or a halogenated alkyl group having 1 to 5 carbon atoms. Ya⁰ represents a single bond or a divalent linking group. Va⁰ represents a single bond or a linear or branched alkylene group. m represents an integer of 1 or greater, and M^{m+} represents an m-valent cation.]

In Formula (m0-1), R has the same definition as that for R in Formula (a0-1).

In Formula (m0-1), Ya⁰ has the same definition as that for Ya⁰ in Formula (a0-1).

In Formula (m0-1), Va⁰ has the same definition as that for Va⁰ in Formula (a0-1).

In Formula (m0-1), M^{m+} has the same definition as that for M^{m+} in Formula (a0-1).

A compound represented by General Formula (m0-1-1) is preferable as the compound (M0).

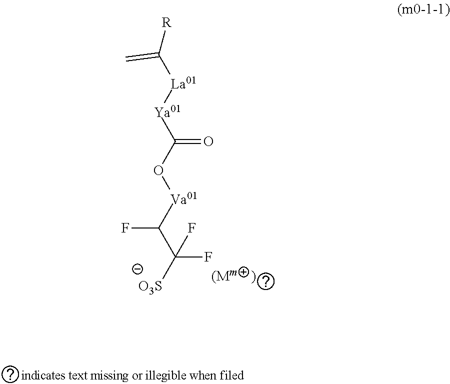

[In the formula, R represents a hydrogen atom, an alkyl group having 1 to 5 carbon atoms, a halogen atom, or a halogenated alkyl group having 1 to 5 carbon atoms. La⁰¹ represents a single bond, —COO—, or an aromatic hydrocarbon group which may have a substituent. Ya⁰¹ represents a single bond or a divalent linking group. Va⁰¹ represents a single bond or a linear or branched alkylene group. m represents an integer of 1 or greater, and M^{m+} represents an m-valent cation.]

In Formula (m0-1-1), R has the same definition as that for R in Formula (a0-1-1).

In Formula (m0-1-1), La⁰¹ has the same definition as that for La⁰¹ in Formula (a0-1-1).

In Formula (m0-1-1), Ya⁰¹ has the same definition as that for Ya⁰¹ in General Formula (a0-1-1).

In Formula (m0-1-1), Va⁰¹ has the same definition as that for Va⁰¹ in Formula (a0-1-1).

In Formula (m0-1-1), M^{m+} has the same definition as that for M^{m+} in Formula (a0-1-1).

Specific examples of the compound (M0) are shown below, but the compound (M0) is not limited thereto. In the formulae shown below, R^{α} represents a hydrogen atom, a methyl group, or a trifluoromethyl group.

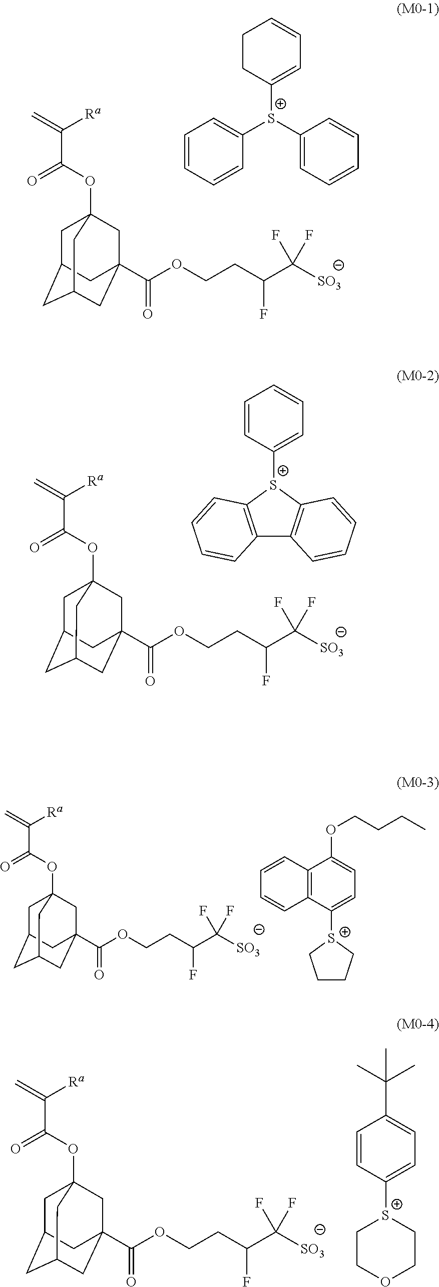

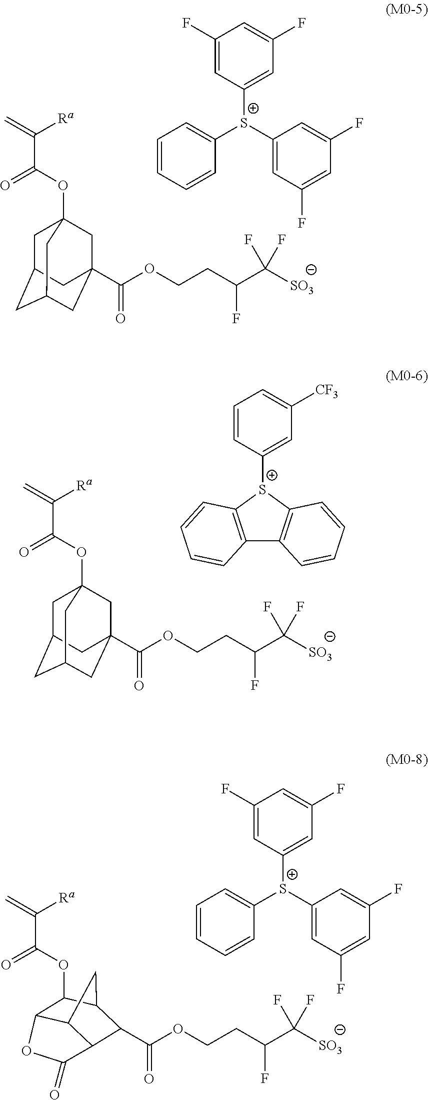

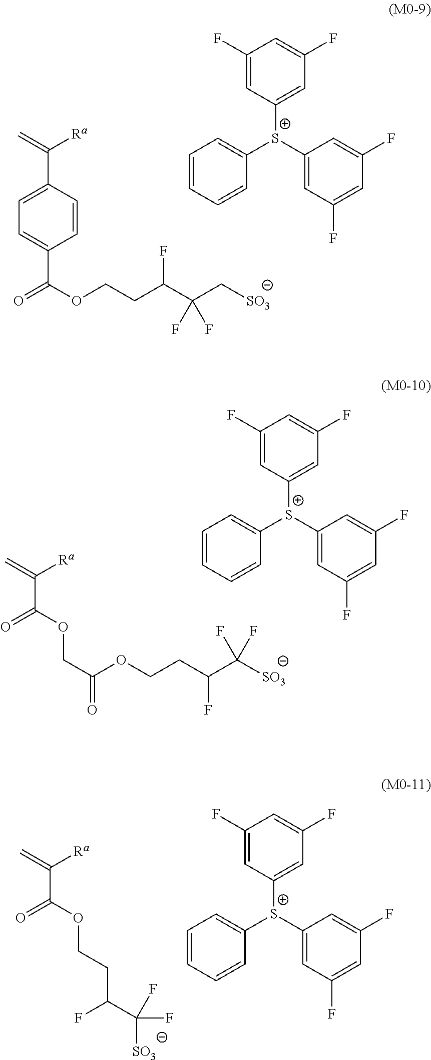

### (Method of Producing Compound (M0))

The compound of the present embodiment can be produced by combining known methods.

For example, a compound represented by General Formula (N0) is obtained by a reaction (hereinafter, also referred to as “reaction (A1)) between a compound represented by General Formula (H01) and a compound represented by General Formula (W01).

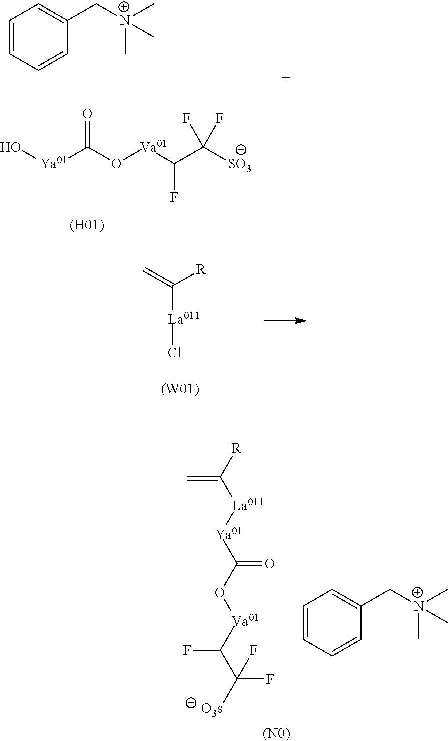

[In the reaction formulae, R represents a hydrogen atom, an alkyl group having 1 to 5 carbon atoms, a halogen atom, or a halogenated alkyl group having 1 to 5 carbon atoms. La⁰¹¹ represents —CO—. La⁰¹ represents —COO—. Ya⁰¹ represents a divalent Va⁰¹¹ represents a single bond or a linear or branched alkylene group.] linking group.

R, Ya⁰¹, Va⁰¹, and M^{m+} in the reaction formulae shown above each has the same definition as that for R, Ya⁰¹, and Va⁰¹ in General Formula (a0-1-1). La⁰¹ represents —COO—, and La⁰¹¹ represents —CO—.

Alternatively, a compound represented by General Formula (N0) is obtained by a reaction (hereinafter, also referred to as “reaction (A2)”) between a compound represented by General Formula (H02) and a compound represented by General Formula (W02).

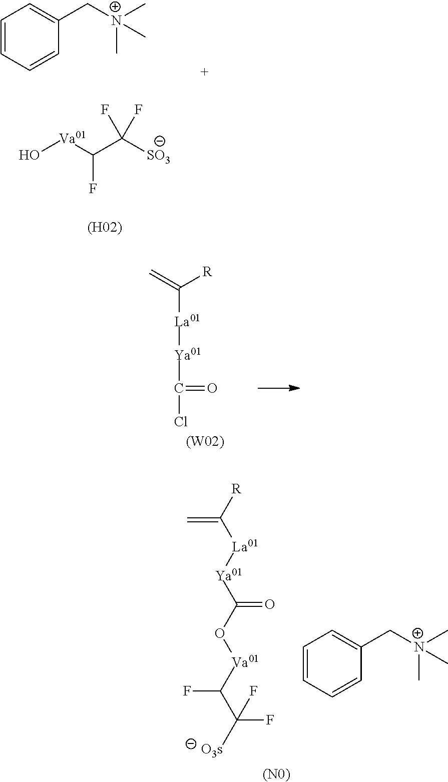

[In the reaction formulae, R represents a hydrogen atom, an alkyl group having 1 to 5 carbon atoms, a halogen atom, or a halogenated alkyl group having 1 to 5 carbon atoms. La⁰¹ represents a single bond or an aromatic hydrocarbon group which may have a substituent. Ya⁰¹ represents a single bond or a divalent linking group. Va⁰¹ represents a single bond or a linear or branched alkylene group.]

R, Ya⁰¹, and Va⁰¹ in the reaction formulae shown above each has the same definition as that for R, Ya⁰¹, and Va⁰ in General Formula (a0-1-1). La⁰¹ represents a single bond or an aromatic hydrocarbon group which may have a substituent.

Next, a compound (M0) can be produced by performing a salt exchange reaction (hereinafter, also referred to as “reaction (B)”) between a compound represented by General Formula (N0) and a compound represented by General Formula (C0). A commercially available product may be used as the above-described compound, or the compound may be synthesized by a known method.

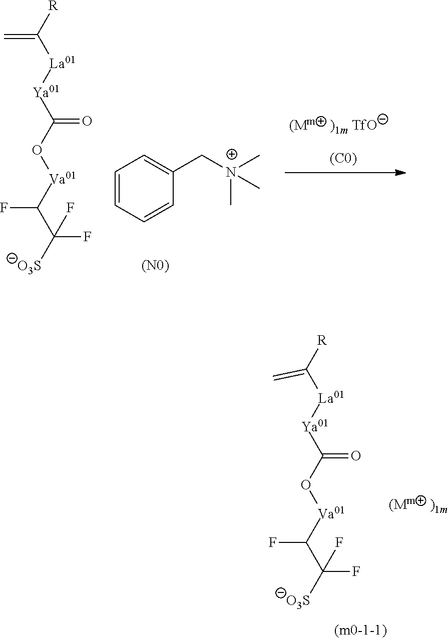

[In the formula, R represents a hydrogen atom, an alkyl group having 1 to 5 carbon atoms, a halogen atom, or a halogenated alkyl group having 1 to 5 carbon atoms. La01 represents —COO— or an aromatic hydrocarbon group which may have a substituent. Ya⁰¹ represents a divalent linking group. Va⁰¹ represents a single bond or a linear or branched alkylene group. m represents an integer of 1 or greater, and M^{m+} represents an m-valent cation.]

R, La⁰¹, Ya⁰¹, Va⁰¹, and M^{m+} in the reaction formulae shown above each has the same definition as that for R, La⁰¹, Ya⁰¹, Va⁰¹, and M^{m+} in General Formula (a0-1-1). TfO⁻ represents a triflate anion.

The temperature conditions for the reaction (A1) and the reaction (A2) are not particularly limited, and are, for example, about −10° C. to 120° C.

The reaction time of the reaction (A1) and the reaction (A2) is not particularly limited, and is, for example, about 1 to 72 hours.

Examples of the reaction solvent used in the above-described reaction (A1) and the above-described reaction (A2) include dichloromethane, dichloroethane, chloroform, tetrahydrofuran, N,N-dimethylformamide, acetonitrile, propionitrile, N,N′-dimethylacetamide, and dimethyl sulfoxide.

In the reaction (A1) and the reaction (A2), a basic catalyst may be used.

Specific examples of the basic catalyst include tertiary amines such as trimethylamine, triethylamine, and tributylamine, aromatic amines such as pyridine, dimethylaminopyridine (DMAP), and pyrrolidinopyridine, diazabicyclononene (DBN), and diazabicycloundecene (DBU).

The temperature condition for the reaction (B) is not particularly limited, and is, for example, about 0° C. to 50° C.

The reaction time of the reaction (B) is not particularly limited, and is, for example, about 1 minute to 24 hours.

As the reaction solvent in the reaction B, for example, a mixed solvent of an organic solvent and water is preferable. Examples of the organic solvent include a ketone-based solvent such as cyclohexanone, methyl ethyl ketone, or diethyl ketone, an ether-based solvent such as diethyl ether, t-butyl methyl ether, or diisopropyl ether, a halogen-based solvent such as tetrahydrofuran, 1,3-dioxolane, dichloromethane (methylene chloride), or 1,2-dichloroethane, an ester-based solvent such as ethyl acetate or propylene glycol monomethyl ether acetate, propionitrile, and a mixed solvent thereof.

After completion of the reaction (B), the compound (M0) in the reaction solution may be isolated and purified.

Known methods of the related art can be used for the isolation and the purification, and for example, any one or a combination of two or more of concentration, solvent extraction, distillation, crystallization, recrystallization, chromatography, and the like can be used.

The structure of the compound obtained as described above can be identified by typical organic analysis methods such as ¹H-nuclear magnetic resonance (NMR) spectroscopy, ¹³C-NMR spectroscopy, ¹⁹F-NMR spectroscopy, infrared (IR) absorption spectroscopy, mass spectrometry (MS), elemental analysis, and X-ray crystal diffraction.

The compound according to the present embodiment can be used for producing a polymer compound according to a fourth aspect described below.

### (Polymer Compound)

The fourth aspect of the present invention relates to a polymer compound having the constitutional unit (a0) represented by General Formula (a0-1).

The polymer compound according to the fourth aspect is the same as the component (A1) described above.

The polymer compound according to the fourth aspect can be used for producing the resist composition according to the first aspect.

### EXAMPLES

Hereinafter, the present invention will be described in more detail based on examples, but the present invention is not limited to these examples.

### Synthesis Example of Compound

### Synthesis Example 1: Compound (M0-1)

The compound (H-1) (5.0 g) and dichloromethane (25 g) were stirred at 0° C., and triethylamine (1.5 g) and methacrylic acid chloride (1.3 g) were added thereto in this order. After the mixture was stirred for 2 hours, the reaction was stopped by adding ultrapure water (25 g). The organic layer was washed with ultrapure water (25 g) three times and concentrated under reduced pressure, thereby obtaining a compound (N-1) (4.5 g) as a light yellow oily substance. The compound (N-1) (4.5 g), a compound (C-1) (3.1 g), dichloromethane (25 g), and ultrapure water (25 g) were stirred at room temperature for liquid separation. The organic layer was washed with ultrapure water (25 g) five times and concentrated under reduced pressure. The concentrated residues were crystallized from dichloromethane/tert-butyl methyl ether, thereby obtaining a compound (M0-1) (4.0 g) as a white solid.

The obtained compound (M0-1) was subjected to NMR measurement, and the structure thereof was identified from the following results.

¹H NMR (DMSO-d6, 400 MHz) d (ppm)=7.74 to 7.90 (m, 15H), 5.95 (s, 1H), 5.59 (s, 1H), 5.11 to 4.89 (m, 1H), 4.27 to 4.09 (m, 2H), 2.51 to 2.35 (m, 1H), 2.29 to 1.94 (m, 9H), 1.83 to 1.58 (m, 9H)

¹⁹F NMR (DMSO-d6, 376 MHz) d (ppm)=−112.5-−113.3 (m, 1F), −121.5-−122.3 (m, 1F), −202.9-−203.3 (m, 1F)

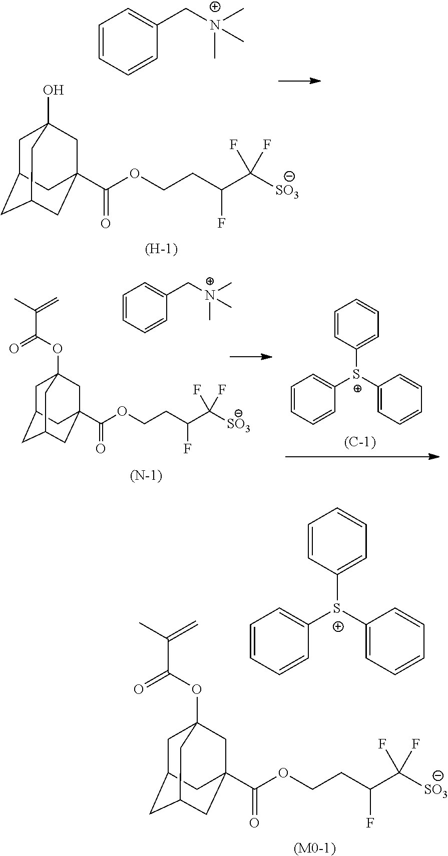

### Synthesis Example 2: Compound (M0-2)

A compound (M0-2) was synthesized by the same method as in Synthesis Example 1 except that a compound (C-2) was used in place of the compound (C-1).

The obtained compound (M0-2) was subjected to NMR measurement, and the structure thereof was identified from the following results.

¹H NMR (DMSO-d6, 400 MHz) d (ppm)=8.50 (d, 2H), 8.37 (d, 2H), 7.93 (t, 2H), 7.55-7.75 (m, 7H), 5.95 (s, 1H), 5.59 (s, 1H), 5.11-4.89 (m, 1H), 4.27-4.09 (m, 2H), 2.51-2.35 (m, 1H), 2.29-1.94 (m, 9H), 1.83-1.58 (m, 9H)

¹⁹F NMR (DMSO-d6, 376 MHz) d (ppm)=−112.5-−113.3 (m, 1F), −121.5-−122.3 (m, 1F), −202.9-−203.3 (m, 1F)

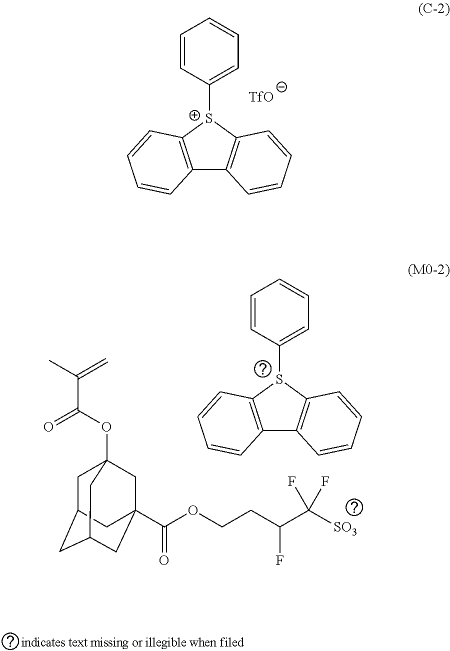

### Synthesis Example 3: Compound (M0-3)

A compound (M0-3) was synthesized by the same method as in Synthesis Example 1 except that a compound (C-3) was used in place of the compound (C-1).

The obtained compound (M0-3) was subjected to NMR measurement, and the structure thereof was identified from the following results.

¹H NMR (DMSO-d6, 400 MHz) d (ppm)=8.53-8.30 (m, 2H), 8.12 (d, 1H), 7.92-7.82 (m, 1H), 7.82-7.68 (m, 1H), 7.20 (d, 1H), 4.31 (t, 2H), 5.95 (s, 1H), 5.59 (s, 1H), 5.11-4.89 (m, 1H), 4.27-4.09 (m, 2H), 4.09-4.00 (m, 2H), 3.81-3.70 (m, 2H), 2.51-2.35 (m, 7H), 2.29-1.94 (m, 9H), 1.83-1.58 (m, 11H), 1.00 (t, 3H)

¹⁹F NMR (DMSO-d6, 376 MHz) d (ppm)=−112.5-−113.3 (m, 1F), −121.5-−122.3 (m, 1F), −202.9-−203.3 (m, 1F)

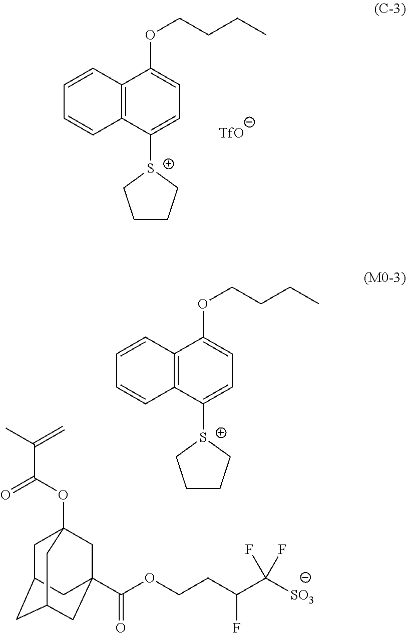

### Synthesis Example 4: Compound (M0-4)

A compound (M0-4) was synthesized by the same method as in Synthesis Example 1 except that a compound (C-4) was used in place of the compound (C-1).

The obtained compound (M0-3) was subjected to NMR measurement, and the structure thereof was identified from the following results.

¹H NMR (DMSO-d6, 400 MHz) d (ppm)=7.98 (d, 2H), 7.76 (d, 2H), 5.95 (s, 1H), 5.59 (s, 1H), 5.11-4.89 (m, 1H), 4.43-3.89 (m, 10H), 2.51-2.35 (m, 1H), 2.29-1.94 (m, 9H), 1.83-1.58 (m, 9H)

¹⁹F NMR (DMSO-d6, 376 MHz) d (ppm)=−112.5-−113.3 (m, 1F), −121.5-−122.3 (m, 1F), −202.9-−203.3 (m, 1F)

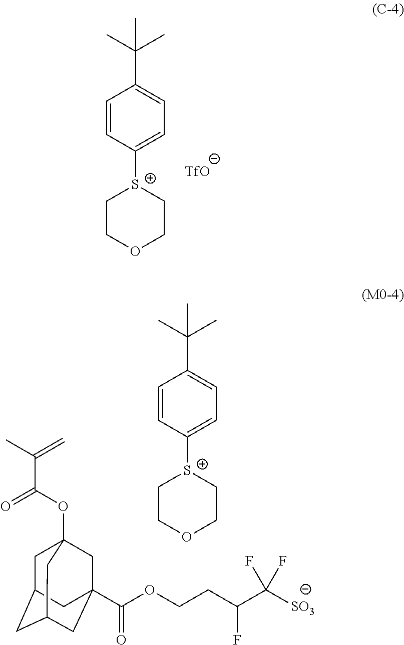

### Synthesis Example 5: Compound (M0-5)

A compound (M0-5) was synthesized by the same method as in Synthesis Example 1 except that a compound (C-5) was used in place of the compound (C-1).

The obtained compound (M0-5) was subjected to NMR measurement, and the structure thereof was identified from the following results.

¹H NMR (DMSO-d6, 400 MHz) d (ppm)=7.77-7.98 (m, 11H), 5.95 (s, 1H), 5.59 (s, 1H), 5.11-4.89 (m, 1H), 4.27-4.09 (m, 2H), 2.51-2.35 (m, 1H), 2.29-1.94 (m, 9H), 1.83-1.58 (m, 9H)

¹⁹F NMR (DMSO-d6, 376 MHz) d (ppm)=−103.9 (s, 4F), −112.5-113.3 (m, 1F), −121.5-122.3 (m, 1F), −202.9-−203.3 (m, 1F)

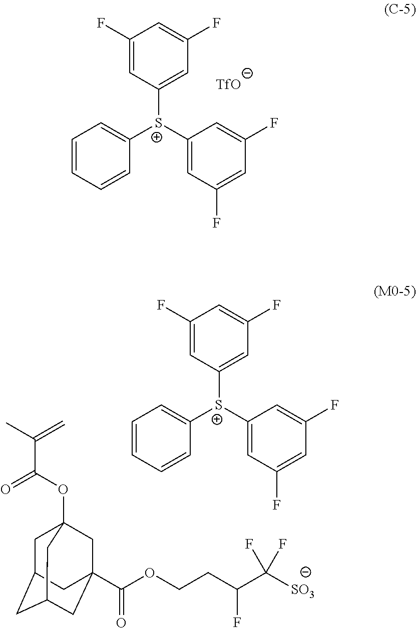

### Synthesis Example 6: Compound (M0-6)

A compound (M0-6) was synthesized by the same method as in Synthesis Example 1 except that a compound (C-6) was used in place of the compound (C-1).

The obtained compound (M0-6) was subjected to NMR measurement, and the structure thereof was identified from the following results.

¹H NMR (DMSO-d6, 400 MHz) d (ppm)=8.60 (s, 1H), 8.55 (d, 2H), 8.41 (d, 2H), 8.08 (d, 1H), 7.98 (t, 2H), 7.78 (t, 2H), 7.70 (t, 1H), 7.34 (d, 1H), 5.95 (s, 1H), 5.59 (s, 1H), 5.11-4.89 (m, 1H), 4.27-4.09 (m, 2H), 2.51-2.35 (m, 1H), 2.29-1.94 (m, 9H), 1.83-1.58 (m, 9H)

¹⁹F NMR (DMSO-d6, 376 MHz) d (ppm)=−61.4 (s, 3F), −112.5-−113.3 (m, 1F), −121.5-−122.3 (m, 1F), −202.9-−203.3 (m, 1F)

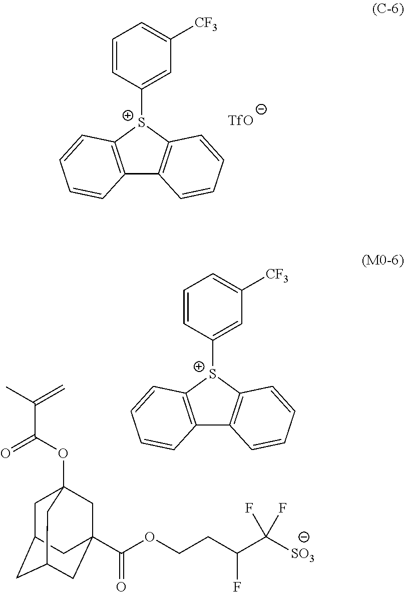

### Synthesis Example 7: Compound (M0-7)

A compound (M0-7) was synthesized by the same method as in Synthesis Example 5 except that 2-trifluoromethylacrylic acid chloride was used in place of methacrylic acid chloride.

The obtained compound (M0-7) was subjected to NMR measurement, and the structure thereof was identified from the following results.

¹H NMR (DMSO-d6, 400 MHz) d (ppm)=7.77-7.98 (m, 11H), 5.95 (s, 1H), 5.59 (s, 1H), 5.11-4.89 (m, 1H), 4.27-4.09 (m, 2H), 2.51-2.35 (m, 1H), 2.29-1.94 (m, 9H), 1.83-1.58 (m, 6H)

¹⁹F NMR (DMSO-d6, 376 MHz) d (ppm)=−103.9 (s, 4F), −112.5-−113.3 (m, 1F), −121.5-−122.3 (m, 1F), −202.9-−203.3 (m, 1F)

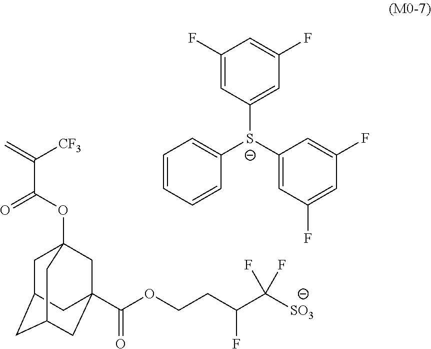

### Synthesis Example 8: Compound (M0-8)

A compound (M0-8) was synthesized by the same method as in Synthesis Example 5 except that a compound (H-2) was used in place of the compound (H-1).

The obtained compound (M0-8) was subjected to NMR measurement, and the structure thereof was identified from the following results.

¹H NMR (DMSO-d6, 400 MHz) d (ppm)=7.77-7.98 (m, 11H), 5.95 (s, 1H), 5.59 (s, 1H), 5.11-4.89 (m, 1H), 4.65-4.61 (m, 2H), 4.27-4.09 (m, 2H), 3.26-3.23 (m, 1H), 2.93-2.76 (m, 3H), 2.51-2.35 (m, 1H), 1.95-1.90 (m, 5H), 1.59 (d, 1H)

¹⁹F NMR (DMSO-d6, 376 MHz) d (ppm)=−103.9 (s, 4F), −112.5-−113.3 (m, 1F), −121.5-−122.3 (m, 1F), −202.9-−203.3 (m, 1F)

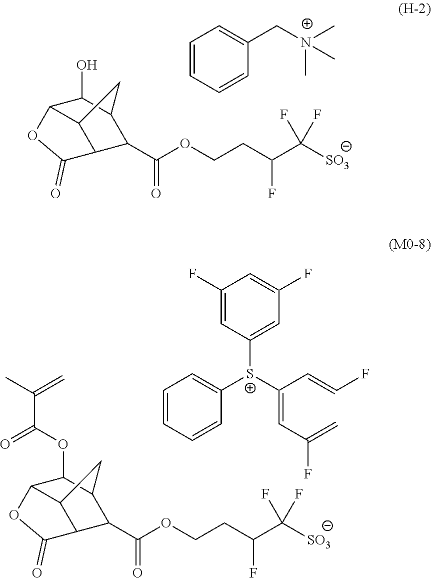

### Synthesis Example 9: Compound (M0-9)

A compound (M0-9) was synthesized by the same method as in Synthesis Example 5 except that a compound (H-3) was used in place of the compound (H-1) and 4-vinylbenzoyl chloride was used in place of methacrylic acid chloride.

The obtained compound (M0-9) was subjected to NMR measurement, and the structure thereof was identified from the following results.

¹H NMR (DMSO-d6, 400 MHz) d (ppm)=7.98-7.77 (m, 13H), 7.60 (s, 2H), 6.85-6.73 (m, 1H), 5.95 (d, 1H), 5.42 (d, 1H), 5.11-4.89 (m, 1H), 4.27-4.09 (m, 2H), 2.51-2.35 (m, 1H), 1.83-1.58 (m, 1H)

¹⁹F NMR (DMSO-d6, 376 MHz) d (ppm)=−103.9 (s, 4F), −112.5-−113.3 (m, 1F), −121.5-−122.3 (m, 1F), −202.9-−203.3 (m, 1F)

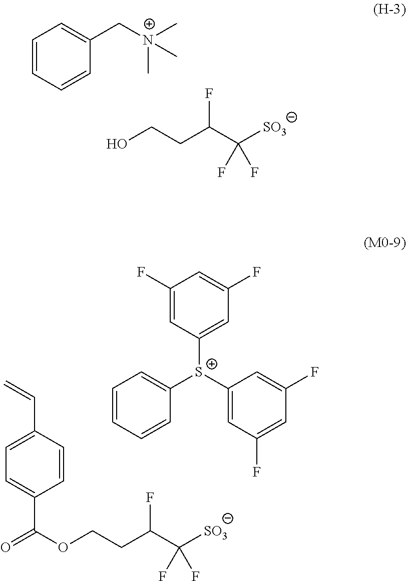

### Synthesis Example 10: Compound (M0-10)

A compound (M0-10) was synthesized by the same method as in Synthesis Example 5 except that a compound (H-4) was used in place of the compound (H-1).

The obtained compound (M0-10) was subjected to NMR measurement, and the structure thereof was identified from the following results.

¹H NMR (DMSO-d6, 400 MHz) d (ppm)=7.77-7.98 (m, 11H), 5.95 (s, 1H), 5.59 (s, 1H), 5.11-4.89 (m, 1H), 4.61 (s, 2H), 4.27-4.09 (m, 2H), 2.51-2.35 (m, 1H), 1.91 (s, 3H), 1.83-1.58 (m, 1H)

¹⁹F NMR (DMSO-d6, 376 MHz) d (ppm)=−103.9 (s, 4F), −112.5-−113.3 (m, 1F), −121.5-122.3 (m, 1F), −202.9-−203.3 (m, 1F)

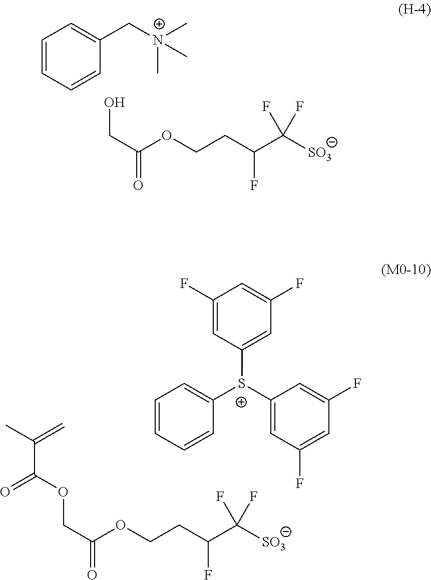

### Synthesis Example 11: Compound (M0-11)

A compound (M0-11) was synthesized by the same method as in Synthesis Example 5 except that a compound (H-3) was used in place of the compound (H-1).

The obtained compound (M0-11) was subjected to NMR measurement, and the structure thereof was identified from the following results.

¹H NMR (DMSO-d6, 400 MHz) d (ppm)=7.77-7.98 (m, 11H), 5.95 (s, 1H), 5.59 (s, 1H), 5.11-4.89 (m, 1H), 4.27-4.09 (m, 2H), 2.51-2.35 (m, 1H), 1.91 (s, 3H), 1.83-1.58 (m, 1H)

¹⁹F NMR (DMSO-d6, 376 MHz) d (ppm)=−103.9 (s, 4F), −112.5-−113.3 (m, 1F), −121.5-122.3 (m, 1F), −202.9-−203.3 (m, 1F)

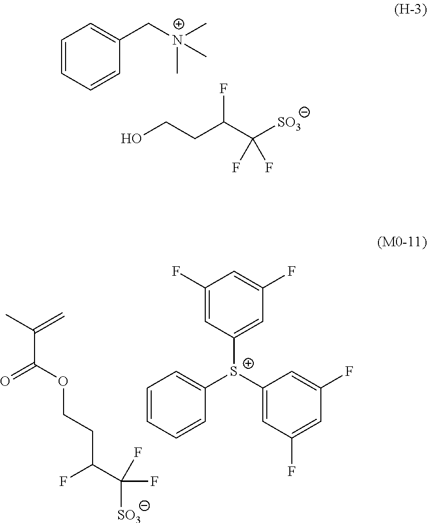

### Synthesis Example of Polymer Compound

### Synthesis Example of Polymer Compound (A1-1)

A dropping solution was prepared by dissolving 4.2 g of the compound (M0-1), 2.6 g of a compound (m-a10-1pre), 3.2 g of a compound (m-a1-1), and 0.5 g of dimethyl azobis(isobutyrate) (V-601) as a polymerization initiator in 15 g of methyl ethyl ketone (MEK). 8.0 g of MEK was added to a three-necked flask connected with a thermometer, a reflux pipe, and a nitrogen introduction pipe, the solution was heated to 85° C. in a nitrogen atmosphere, and the above-described dropping solution was added dropwise thereto over 4 hours. After completion of the dropwise addition, the reaction solution was stirred at 85° C. for 1 hour. Then, the reaction solution was cooled to room temperature. After completion of the reaction, the obtained reaction solution was precipitated with 100 g of heptane, and the precipitate was washed. The obtained white solid was filtered and dried under reduced pressure overnight, thereby obtaining a polymer compound (A1-1).

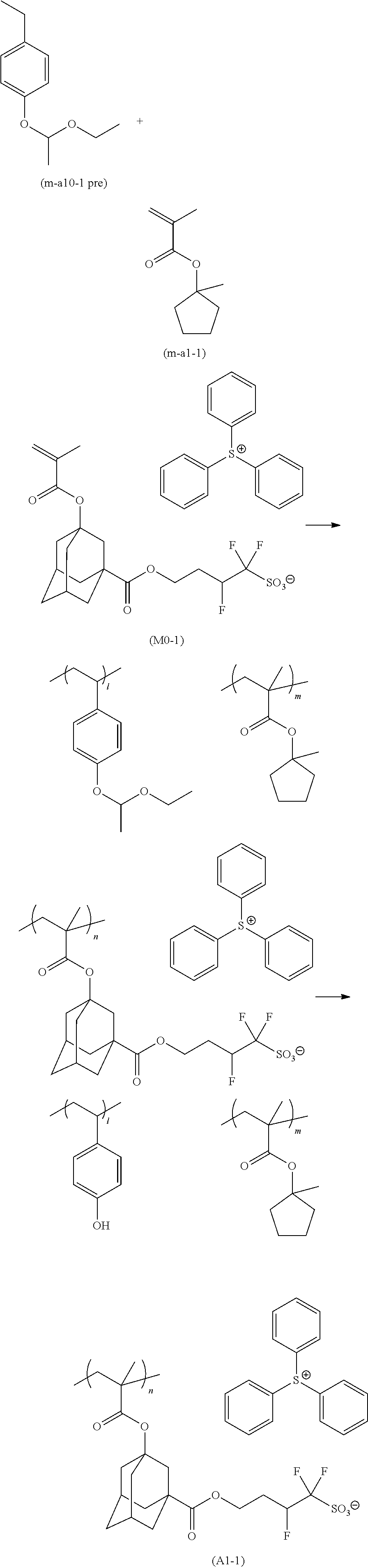

### Synthesis Examples of Polymer Compounds (A1-2) to (A1-19)

Polymer compounds (A1-2) to (A1-19) were synthesized by the same method as in the synthesis example of the polymer compound (A1-1) except that the monomer used and the amount thereof used were changed. The compounds (M0-1) to (M0-11) were used as monomers for deriving the constitutional unit (a0).

The weight-average molecular weight (Mw) and the polydispersity (Mw/Mn) of the polymer compounds (A1-1) to (A1-19) were determined by GPC measurement (in terms of standard polystyrene). The copolymer compositional ratio (ratio (molar ratio) between constitutional units in structural formula) of the polymer compounds (A1-1) to (A1-19) was determined by the carbon 13 nuclear magnetic resonance spectrum (600 MHz_¹³C-NMR).
Polymer compound (A1-1): weight-average molecular weight (Mw) of 19900, polydispersity (Mw/Mn) of 1.69, 1/m/n=35/50/15
Polymer compound (A1-2): weight-average molecular weight (Mw) of 19900, polydispersity (Mw/Mn) of 1.69, 1/m/n=35/50/15
Polymer compound (A1-3): weight-average molecular weight (Mw) of 19900, polydispersity (Mw/Mn) of 1.69, 1/m/n=35/50/15
Polymer compound (A1-4): weight-average molecular weight (Mw) of 19900, polydispersity (Mw/Mn) of 1.69, 1/m/n=35/50/15
Polymer compound (A1-5): weight-average molecular weight (Mw) of 19900, polydispersity (Mw/Mn) of 1.69, 1/m/n=35/50/15
Polymer compound (A1-6): weight-average molecular weight (Mw) of 19900, polydispersity (Mw/Mn) of 1.69, 1/m/n=35/50/15
Polymer compound (A1-7): weight-average molecular weight (Mw) of 19900, polydispersity (Mw/Mn) of 1.69, 1/m/n=35/50/15
Polymer compound (A1-8): weight-average molecular weight (Mw) of 19900, polydispersity (Mw/Mn) of 1.69, 1/m/n=35/50/15
Polymer compound (A1-9): weight-average molecular weight (Mw) of 19900, polydispersity (Mw/Mn) of 1.69, 1/m/n=35/50/15
Polymer compound (A1-10): weight-average molecular weight (Mw) of 19900, polydispersity (Mw/Mn) of 1.69, 1/m/n=35/50/15
Polymer compound (A1-11): weight-average molecular weight (Mw) of 19900, polydispersity (Mw/Mn) of 1.69, 1/m/n=35/50/15
Polymer compound (A1-12): weight-average molecular weight (Mw) of 19900, polydispersity (Mw/Mn) of 1.69, 1/m/n=35/50/15
Polymer compound (A1-13): weight-average molecular weight (Mw) of 19900, polydispersity (Mw/Mn) of 1.69, 1/m/n=35/50/15
Polymer compound (A1-14): weight-average molecular weight (Mw) of 19900, polydispersity (Mw/Mn) of 1.69, 1/m/n=39/59/2
Polymer compound (A1-15): weight-average molecular weight (Mw) of 19900, polydispersity (Mw/Mn) of 1.69, 1/m/n=38/57/5
Polymer compound (A1-16): weight-average molecular weight (Mw) of 19900, polydispersity (Mw/Mn) of 1.69, 1/m/n=36/54/10
Polymer compound (A1-17): weight-average molecular weight (Mw) of 19900, polydispersity (Mw/Mn) of 1.69, 1/m/n=30/45/25
Polymer compound (A1-18): weight-average molecular weight (Mw) of 19900, polydispersity (Mw/Mn) of 1.69, 1/m/n=28/42/30
Polymer compound (A1-19): weight-average molecular weight (Mw) of 19900, polydispersity (Mw/Mn) of 1.69, 1/m/n=26/39/35

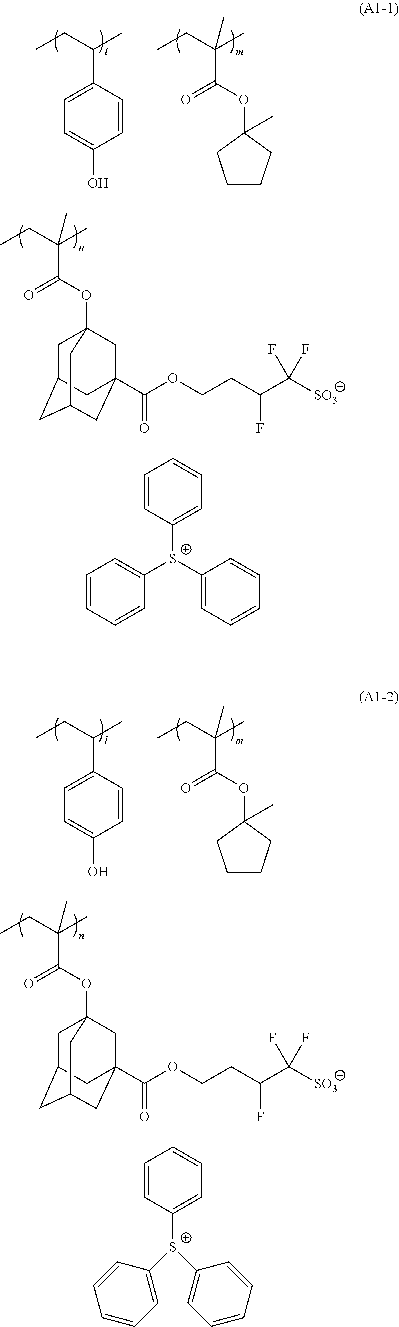

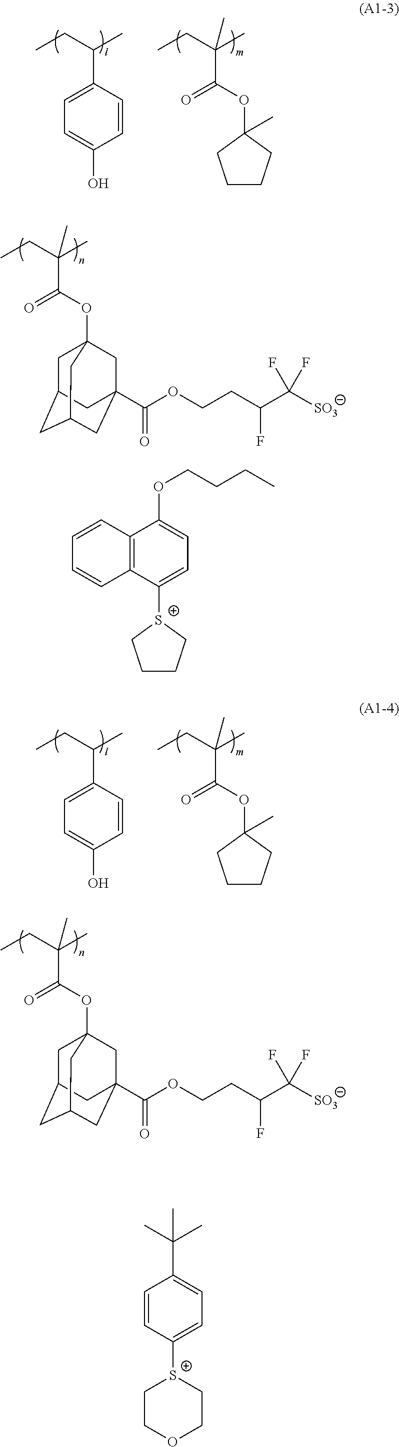

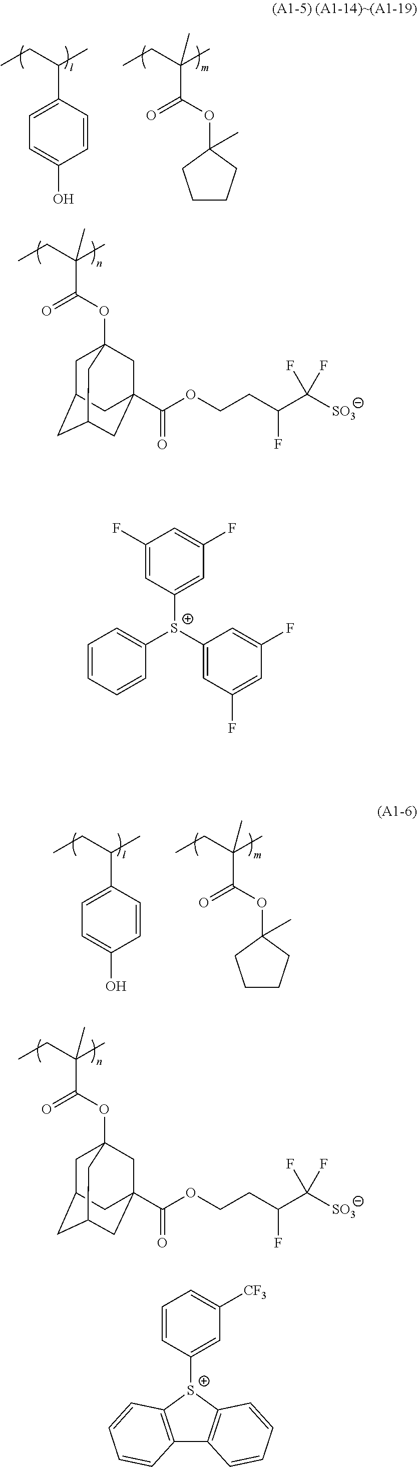

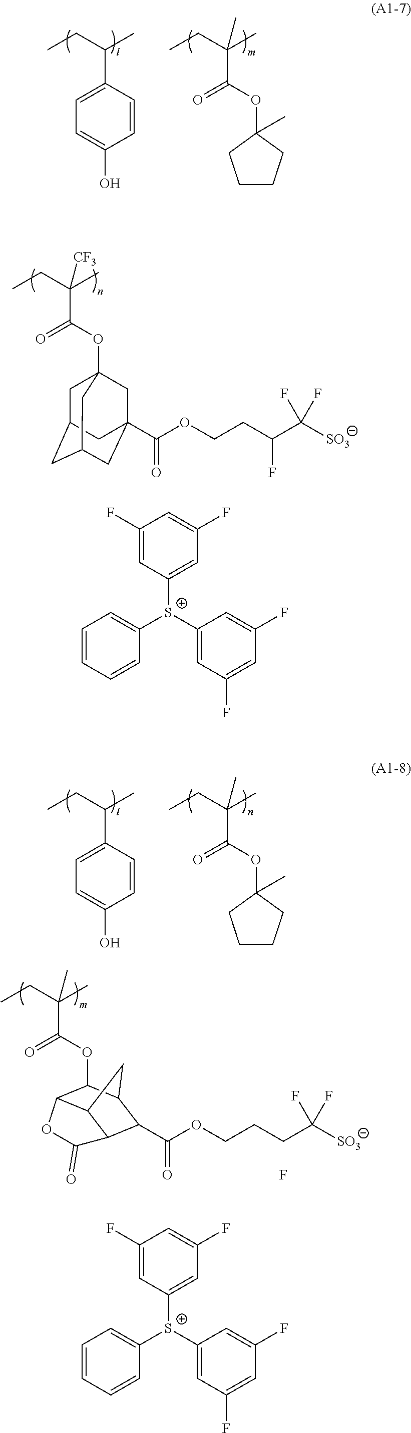

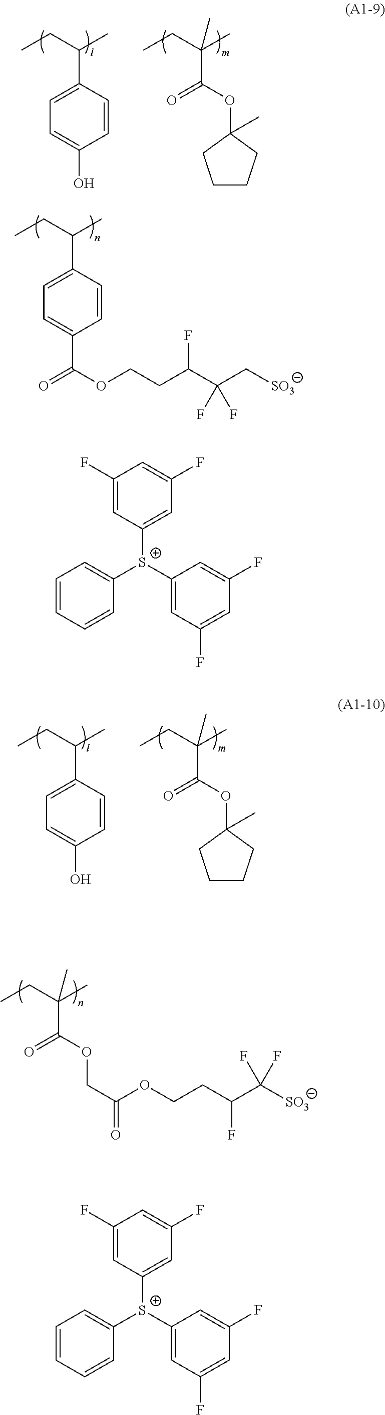

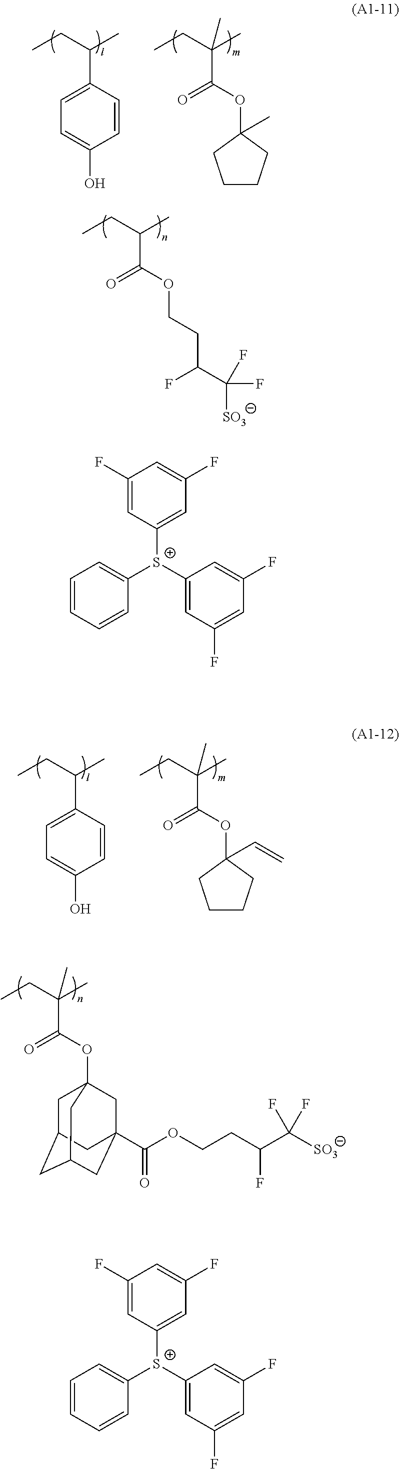

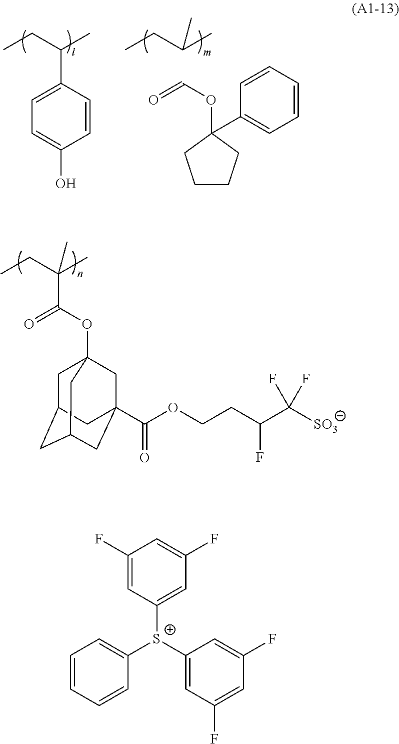

### <Preparation of Resist Composition>

### Examples 1 to 19 and Comparative Examples 1 to 8

Each of the components shown in Tables 1 and 2 was mixed and dissolved to prepare a resist composition of each Example.

TABLE 1

| | Component | Component | Component | Component |
|---|---|---|---|---|
| | (A) | (B) | (D) | (S) |
| Example | (A1)-1 | — | (D)-1 | (S)-1 |
| 1 | [100] | | [4.2] | [8000] |
| Example | (A1)-2 | — | (D)-1 | (S)-1 |
| 2 | [100] | | [4.2] | [8000] |
| Example | (A1)-3 | — | (D)-1 | (S)-1 |
| 3 | [100] | | [4.2] | [8000] |
| Example | (A1)-4 | — | (D)-1 | (S)-1 |
| 4 | [100] | | [4.2] | [8000] |
| Example | (A1)-5 | — | (D)-1 | (S)-1 |
| 5 | [100] | | [4.2] | [8000] |
| Example | (A1)-6 | — | (D)-1 | (S)-1 |
| 6 | [100] | | [4.2] | [8000] |
| Example | (A1)-7 | — | (D)-1 | (S)-1 |
| 7 | [100] | | [4.2] | [8000] |
| Example | (A1)-8 | — | (D)-1 | (S)-1 |
| 8 | [100] | | [4.2] | [8000] |
| Example | (A1)-9 | — | (D)-1 | (S)-1 |
| 9 | [100] | | [4.2] | [8000] |
| Example | (A1)-10 | — | (D)-1 | (S)-1 |
| 10 | [100] | | [4.2] | [8000] |
| Example | (A1)-11 | — | (D)-1 | (S)-1 |
| 11 | [100] | | [4.2] | [8000] |
| Example | (A1)-12 | — | (D)-1 | (S)-1 |
| 12 | [100] | | [4.2] | [8000] |
| Example | (A1)-13 | — | (D)-1 | (S)-1 |
| 13 | [100] | | [4.2] | [8000] |
| Example | (A1)-14 | — | (D)-1 | (S)-1 |
| 14 | [100] | | [4.2] | [8000] |
| Example | (A1)-15 | — | (D)-1 | (S)-1 |
| 15 | [100] | | [4.2] | [8000] |
| Example | (A1)-16 | — | (D)-1 | (S)-1 |
| 16 | [100] | | [4.2] | [8000] |
| Example | (A1)-17 | — | (D)-1 | (S)-1 |
| 17 | [100] | | (4.2] | [8000] |
| Example | (A1)-18 | — | (D)-1 | (S)-1 |
| 18 | [100] | | [4.2] | [8000] |
| Example | (A1)-19 | — | (D)-1 | (S)-1 |
| 19 | [100] | | [4.2] | [8000] |

TABLE 2

| | Component | Component | Component | Component |
|---|---|---|---|---|
| | (A) | (B) | (D) | (S) |
| Comparative | (A2)-1 | — | (D)-1 | (S)-1 |
| Example 1 | [100] | | [4.2] | [8000] |
| Comparative | (A2)-2 | — | (D)-1 | (S)-1 |
| Example 2 | [100] | | [4.2] | [8000] |
| Comparative | (A2)-3 | — | (D)-1 | (S)-1 |
| Example 3 | [100] | | [4.2] | [8000] |
| Comparative | (A2)-4 | — | (D)-1 | (S)-1 |
| Example 4 | [100] | | [4.2] | [8000] |
| Comparative | (A2)-5 | — | (D)-1 | (S)-1 |
| Example 5 | [100] | | [4.2] | [8000] |
| Comparative | (A2)-6 | — | (D)-1 | (S)-1 |
| Example 6 | [100] | | [4.2] | [8000] |
| Comparative | (A2)-7 | (B)-1 | (D)-1 | (S)-1 |
| Example 7 | [100] | [18.5] | [4.2] | [8000] |
| Comparative | (A2)-8 | — | (D)-1 | (S)-1 |
| Example 8 | [100] | | [4.2] | [8000] |

In Tables 1 and 2, each abbreviation has the following meaning. The numerical values in the brackets are blending amounts (parts by mass).
(A1)-1 to (A1)-19: polymer compounds (A1-1) to (A1-19) shown above
(A2)-1: polymer compound (A2-1) shown below. Weight-average molecular weight (Mw) of 19900, polydispersity (Mw/Mn) of 1.69, 1/m/n=35/50/15
(A2)-2: polymer compound (A2-2) shown below. Weight-average molecular weight (Mw) of 19900, polydispersity (Mw/Mn) of 1.69, 1/m/n=35/50/15
(A2)-3: polymer compound (A2-3) shown below. Weight-average molecular weight (Mw) of 19900, polydispersity (Mw/Mn) of 1.69, 1/m/n=35/50/15
(A2)-4: polymer compound (A2-4) shown below. Weight-average molecular weight (Mw) of 19900, polydispersity (Mw/Mn) of 1.69, 1/m/n=35/50/15
(A2)-5: polymer compound (A2-5) shown below. Weight-average molecular weight (Mw) of 19900, polydispersity (Mw/Mn) of 1.69, 1/m/n=35/50/15
(A2)-6: polymer compound (A2-6) shown below. Weight-average molecular weight (Mw) of 19900, polydispersity (Mw/Mn) of 1.69, 1/m/n=35/50/15
(A2)-7: polymer compound (A2-7) shown below. Weight-average molecular weight (Mw) of 19900, polydispersity (Mw/Mn) of 1.69, 1/m=40/60
(A2)-8: polymer compound (A2-8) shown below. Weight-average molecular weight (Mw) of 19900, polydispersity (Mw/Mn) of 1.69, 1/m/n=35/50/15

The weight-average molecular weight (Mw) is a weight-average molecular weight in terms of standard polystyrene, which is determined by GPC measurement. The copolymer compositional ratio (ratio (molar ratio) between constitutional units in structural formula) was determined by ¹³C-NMR.

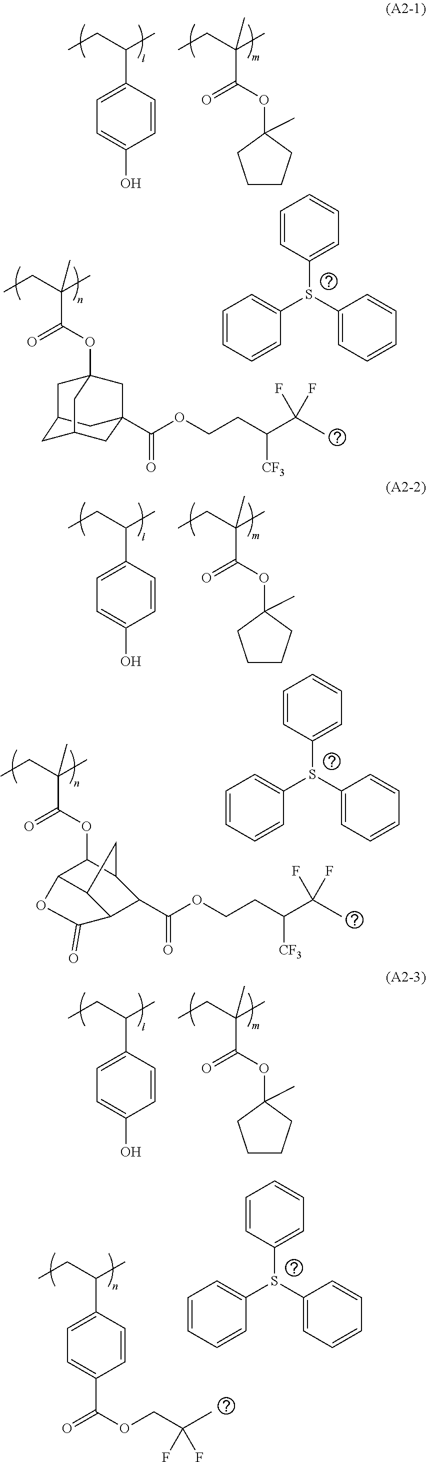

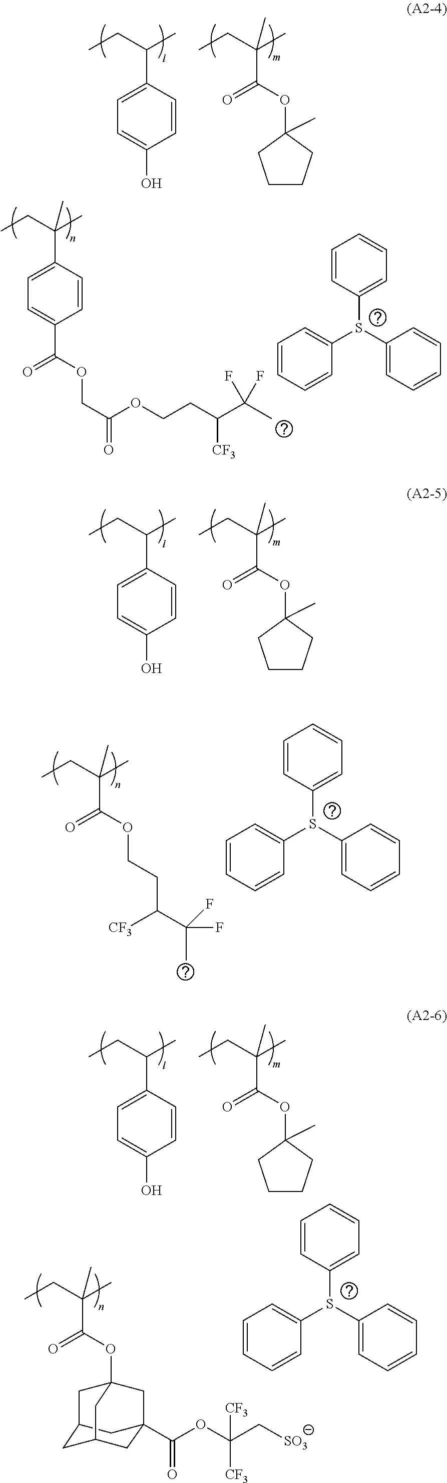

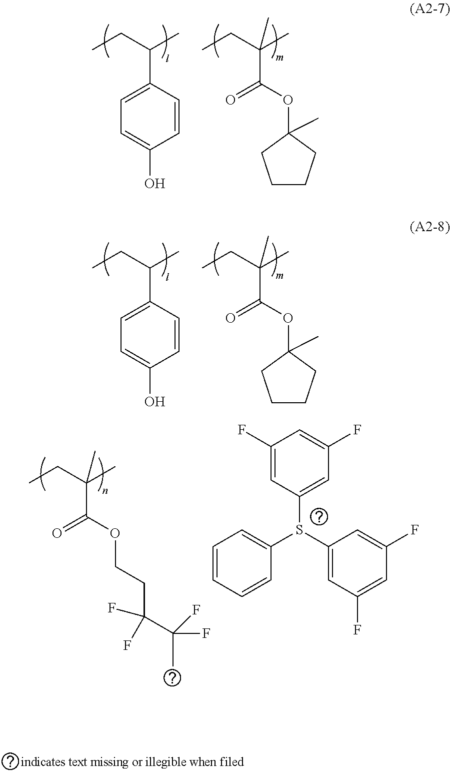

(B)-1: acid generator consisting of compound (B-1) shown below
(D)-1: acid diffusion control agent consisting of compound (D-2) shown below

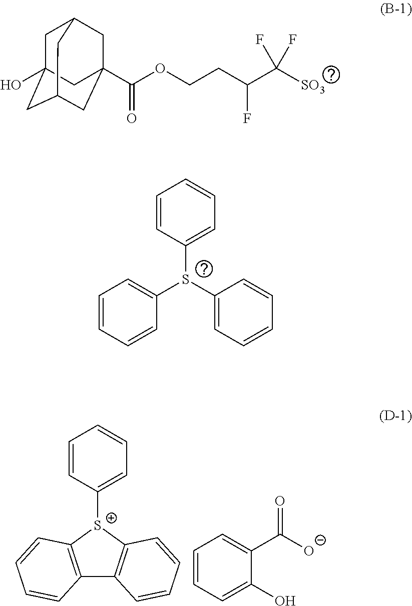

(S)-1: mixed solvent of propylene glycol monomethyl ether acetate/propylene glycol monomethyl ether=60/40 (mass ratio)

### <Resist Pattern Formation>

Step (i): An 8-inch silicon substrate which had been subjected to hexamethyldisilazane (HMDS) treatment was coated with the resist composition of each example using a spinner, and subjected to a pre-bake (PAB) treatment on a hot plate at a temperature of 110° C. for 60 seconds so that the 8-inch silicon substrate was dried, thereby forming a resist film having a film thickness of 30 nm.
Step (ii): Next, drawing (light exposure) was performed on the resist film using an electron beam lithography apparatus JEOL-JBX-9300FS (manufactured by JEOL Ltd.), with the target size being set to a 1:1 line-and-space pattern (hereinafter, referred to as an “LS pattern”) with a line width of 50 nm, at an acceleration voltage of 100 kV. Thereafter, a post-exposure bake (PEB) treatment was performed thereon at 90° C. for 60 seconds.
Step (iii): Next, alkali development was performed for 60 seconds using a 2.38 mass % tetramethylammonium hydroxide (TMAH) aqueous solution “NMD-3” (trade name, manufactured by Tokyo Obka Kogyo Co., Ltd.) at 23° C.

Thereafter, water rinsing was carried out with pure water for 15 seconds. As a result, a 1:1 LS pattern having a line width of 50 nm was formed.

### [Evaluation of Optimum Exposure Amount (Eop)]

According to <resist pattern formation> described above, an optimum exposure amount Eop (μC/cm²) in which a pattern having the target size was formed was determined. The results are listed in the columns of “Eop (μC/cm²)” in Table 2.

### [Evaluation of Line Width Roughness (LWR)]

Using the LS pattern formed in the section of “resist pattern formation”, the 3σ which is the scale that indicates the LWR was acquired. Here, “3σ ” denotes the triple value (3σ) (unit: nm) of the standard deviation (σ) determined from measurement results obtained by measuring 400 line positions in the longitudinal direction of the line with a scanning electron microscope (trade name: S-9380, manufactured by Hitachi High-Tech Corporation, acceleration voltage of 800 V). In a case where the value of the 3σ is small, this indicates that the roughness of a line side wall is small and an LS pattern with a uniform width is obtained. The results are listed in the columns of “LWR” in Tables 3 and 4.

### [Evaluation of LS Pattern Shape]

The cross-sectional shape of the resist pattern resolved at the optimum exposure amount was observed with a CD-SEM (scanning electron microscope, acceleration voltage: 800 V, trade name: SU-8000, manufactured by Hitachi High-Tech Corporation). A line width Lb at the center of the resist pattern in the height direction and a line width La at the upper part of the resist pattern were measured, and the value of La/Lb was calculated. This value was used as an index for evaluating the LS pattern shape. The rectangularity of the pattern was evaluated as satisfactory in a case where 0.9≤(La/Lb)≤1.1 was satisfied and evaluated as poor otherwise. The values of La/Lb are listed in the columns of “pattern shape” in Tables 3 and 4.

### [Evaluation of Coating Defects]

A resist film having a film thickness of 30 nm was formed in the same manner as in the step (i) of <resist pattern formation>. Foreign matter having a size of 80 nm or greater which had been generated on the surface of the resist film and the number of defects were measured using Surfscan SP2 (product name) (manufactured by KLA Corporation). The measurement result was calculated as a relative value in a case where the number of defects in a case of using the resist composition of Comparative Example 1 was set as a reference (1.0). The results are listed in the columns of “coating defects” in Tables 3 and 4.

TABLE 3

| | Eop | LWR | Pattern | Coating |
|---|---|---|---|---|
| | (μC/cm²) | (nm) | shape | defects |
| Example 1 | 85 | 4.1 | 0.97 | 0.2 |
| Example 2 | 85 | 4.1 | 1.04 | 0.2 |
| Example 3 | 84 | 4.1 | 0.97 | 0.3 |
| Example 4 | 84 | 4.1 | 1.03 | 0.3 |
| Example 5 | 79 | 3.5 | 1.00 | 0.1 |
| Example 6 | 79 | 3.5 | 1.00 | 0.1 |
| Example 7 | 83 | 4.0 | 1.01 | 0.2 |
| Example 8 | 80 | 3.8 | 1.00 | 0.2 |
| Example 9 | 80 | 3.9 | 0.99 | 0.2 |
| Example 10 | 79 | 4.0 | 1.01 | 0.2 |
| Example 11 | 78 | 3.9 | 1.00 | 0.2 |
| Example 12 | 76 | 3.4 | 1.01 | 0.1 |
| Example 13 | 77 | 3.5 | 1.00 | 0.1 |
| Example 14 | 91 | 3.9 | 1.00 | 0.3 |
| Example 15 | 82 | 4.1 | 0.99 | 0.2 |
| Example 16 | 80 | 3.6 | 0.99 | 0.1 |
| Example 17 | 78 | 3.6 | 1.01 | 0.2 |
| Example 18 | 75 | 4.2 | 0.96 | 0.2 |
| Example 19 | 71 | 4.5 | 0.91 | 0.7 |

TABLE 4

| | Eop | LWR | Pattern | Coating |
|---|---|---|---|---|
| | (μC/cm²) | (nm) | shape | defects |
| Comparative | 96 | 4.6 | 0.85 | 1.0 |
| Example 1 | | | | |
| Comparative | 96 | 4.7 | 0.86 | 1.0 |
| Example 2 | | | | |
| Comparative | 96 | 4.7 | 0.85 | 1.1 |
| Example 3 | | | | |
| Comparative | 95 | 4.7 | 1.15 | 1.2 |
| Example 4 | | | | |
| Comparative | 95 | 4.6 | 1.14 | 1.2 |
| Example 5 | | | | |
| Comparative | 97 | 4.8 | 0.83 | 1.2 |
| Example 6 | | | | |
| Comparative | 96 | 4.6 | 1.16 | 1.1 |
| Example 7 | | | | |
| Comparative | 95 | 4.6 | 1.13 | 1.2 |
| Example 8 | | | | |

As listed in Tables 3 and 4, the resist compositions of the examples were all satisfactory in the results of the sensitivity, the LWR, the pattern shape, and the coating defects as compared with the resist compositions of the comparative examples. All the resist compositions of the examples were evaluated as having a satisfactory pattern shape. On the contrary, all the resist compositions of the comparative examples were evaluated as having a poor pattern shape. Since the resin having a constitutional unit that had an anion moiety with two or four fluorine atoms was used in the resist compositions of Comparative Examples 3 and 8, it was found that the resist compositions of the examples exhibited the above-described effects due to having the constitutional unit that had an anion moiety with three fluorine atoms.

While preferred embodiments of the present invention have been described and illustrated above, it should be understood that these are exemplary of the present invention and are not to be considered as limiting. Additions, omissions, substitutions, and other modifications can be made without departing from the scope of the invention. Accordingly, the present invention is not to be considered as being limited by the foregoing description and is only limited by the scope of the appended claims.

What is claimed is:

1. A resist composition which generates an acid upon light exposure and whose solubility in a developing solution is changed by an action of an acid, the resist composition comprising:
a resin component (A1) whose solubility in a developing solution is changed by an action of an acid,
wherein the resin component (A1) has a constitutional unit (a0) represented by General Formula (a0-1), 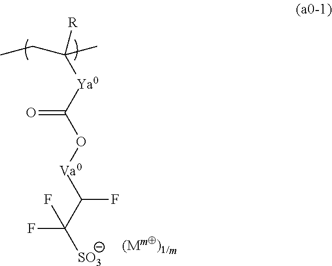
wherein R represents a hydrogen atom, an alkyl group having 1 to 5 carbon atoms, a halogen atom, or a halogenated alkyl group having 1 to 5 carbon atoms, Ya⁰ represents a single bond or a divalent linking group, Va⁰ represents a single bond or a linear or branched alkylene group, m represents an integer of 1 or greater, and M^{m+} represents an m-valent cation.

2. The resist composition according to claim 1,
wherein the constitutional unit (a0) is a constitutional unit represented by General Formula (a0-1-1), 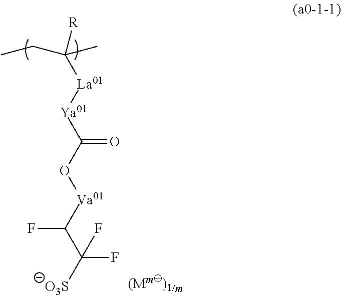
wherein R represents a hydrogen atom, an alkyl group having 1 to 5 carbon atoms, a halogen atom, or a halogenated alkyl group having 1 to 5 carbon atoms, La⁰¹ represents a single bond, —COO—, or an aromatic hydrocarbon group which may have a substituent, Ya⁰¹ represents a single bond or a divalent linking group, Va⁰¹ represents a single bond or a linear or branched alkylene group, m represents an integer of 1 or greater, and M^{m+} represents an m-valent cation.

3. The resist composition according to claim 2, wherein Ya⁰¹ represents a divalent linking group having a polycyclic structure.

4. The resist composition according to claim 1, wherein M^{m+} represents an m-valent cation having a fluorine atom.

5. The resist composition according to claim 1, further comprising an acid diffusion control agent component (D).

6. A resist pattern formation method, comprising:
forming a resist film on a support using the resist composition according to claim 1;
exposing the resist film to light; and
developing the resist film exposed to light to form a resist pattern.

7. A compound which is represented by General Formula (m0-1), 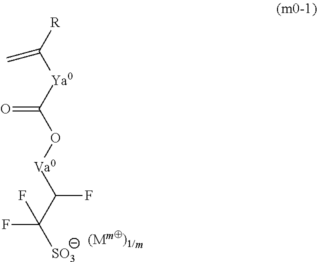
wherein R represents a hydrogen atom, an alkyl group having 1 to 5 carbon atoms, a halogen atom, or a halogenated alkyl group having 1 to 5 carbon atoms, Ya⁰ represents a single bond or a divalent linking group, Va⁰ represents a single bond or a linear or branched alkylene group, m represents an integer of 1 or greater, and M^{m+} represents an m-valent cation.

8. The compound according to claim 7, wherein the compound is a compound represented by General Formula (m0-1-1), 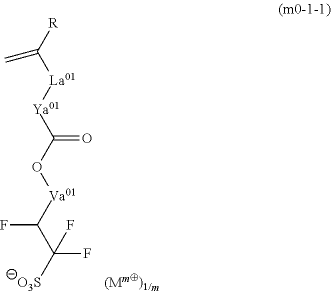
wherein R represents a hydrogen atom, an alkyl group having 1 to 5 carbon atoms, a halogen atom, or a halogenated alkyl group having 1 to 5 carbon atoms, La⁰¹ represents a single bond, —COO—, or an aromatic hydrocarbon group which may have a substituent, Ya⁰¹ represents a single bond or a divalent linking group, Va⁰¹ represents a single bond or a linear or branched alkylene group, m represents an integer of 1 or greater, and M^{m+} represents an m-valent cation.

9. A polymer compound which has a constitutional unit (a0) represented by General Formula (a0-1), 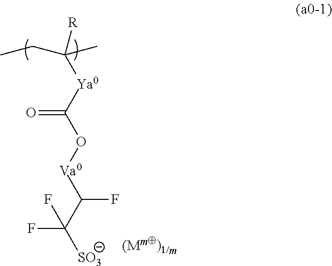
wherein R represents a hydrogen atom, an alkyl group having 1 to 5 carbon atoms, a halogen atom, or a halogenated alkyl group having 1 to 5 carbon atoms, Ya⁰ represents a single bond or a divalent linking group, Va⁰ represents a single bond or a linear or branched alkylene group, m represents an integer of 1 or greater, and M^{m+} represents an m-valent cation.

10. The polymer compound according to claim 9, wherein the constitutional unit (a0) is a constitutional unit represented by General Formula (a0-1-1), 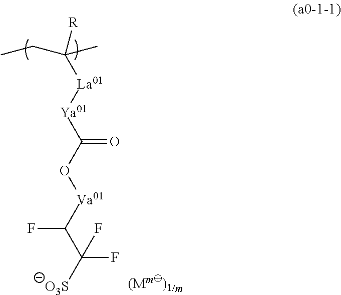
wherein R represents a hydrogen atom, an alkyl group having 1 to 5 carbon atoms, a halogen atom, or a halogenated alkyl group having 1 to 5 carbon atoms, La⁰¹ represents a single bond, —COO—, or an aromatic hydrocarbon group which may have a substituent, Ya⁰¹ represents a single bond or a divalent linking group, Va⁰¹ represents a single bond or a linear or branched alkylene group, m represents an integer of 1 or greater, and M^{m+} represents an m-valent cation.
